(19) United States
(12) Patent Application Publication
(10) Pub. No.: US 2012/0009623 A1
(43) Pub. Date: Jan. 12, 2012

(54) Methods for Recombinant Manufacturing of Anti-RSV Antibodies

(75) Inventors: Anne B. Tolstrup, Hillerod (DK); Johan Lantto, Lund (SE); Finn C. Wiberg, Farum (DK); Lars S. Nielsen, Nivaa (DK)

(73) Assignee: Symphogen A/S

(21) Appl. No.: 13/243,768

(22) Filed: Sep. 23, 2011

Related U.S. Application Data
(63) Continuation of application No. 12/230,885, filed on Sep. 5, 2008, now abandoned.
(60) Provisional application No. 60/971,404, filed on Sep. 11, 2007.

(30) Foreign Application Priority Data
Sep. 7, 2007 (DK) PA 2007 01289

(51) Int. Cl. C12P 21/06; C12N 15/82; C12N 15/80; C12N 5/10; C12N 1/15; C12N 1/19; C12N 15/85; C12N 15/74

(52) U.S. Cl. 435/69.6; 435/455; 435/468; 435/471; 435/339; 435/419; 435/254.11; 435/254.2

(57) ABSTRACT

The invention relates to a method for manufacturing recombinant anti-RSV antibodies and antibody compositions. The method comprises obtaining a collection of cells transfected with a collection of variant nucleic acid sequences, wherein each cell in the collection is transfected with and capable of expressing one distinct anti-RSV antibody. The cells are cultured under suitable conditions for expression of the anti-RSV antibody/antibodies. The nucleic acid sequence is introduced into the cells by transfection with expression vectors, which avoid site-specific integration. The present method is suitable for manufacturing recombinant mono- and polyclonal anti-RSV antibodies for therapeutic uses.

### CROSS REFERENCE TO RELATED APPLICATIONS

This application is a continuation of U.S. application Ser. No. 12/230,885, filed Sep. 5, 2008, which claims the benefit of the filing date of U.S. Provisional Appl. No. 60/971,404, filed Sep. 11, 2007 and Danish Appl. No. PA 2007 01289, filed Sep. 7, 2007, all of which are incorporated by reference in their entireties.

### REFERENCE TO SEQUENCE LISTING SUBMITTED ELECTRONICALLY

The content of the electronically submitted sequence listing Name: 2488.0110002SequenceIDListing.ascii.txt; Size: 308,934 bytes; and Date of Creation: Sep. 22, 2011) filed with the application is incorporated herein by reference in its entirety.

### BACKGROUND OF THE INVENTION

The present invention relates to the manufacture of recombinant anti-RSV antibodies, using production systems which are independent of site-specific integration.

### FIELD OF THE INVENTION

Recombinant polyclonal antibodies may be generated by isolating antibody encoding nucleic acids from donors with an immune response against the desired target, followed by screening for antibodies which specifically bind the desired target. The polyclonal antibody may be manufactured in one vessel by an adapted mammalian expression technology, which is based on site-specific integration of one antibody expression plasmid into the same genomic site of each cell as described in WO 2004/061104. One example of this type of polyclonal antibodies is a recombinant polyclonal antibody against Rhesus D (WO 2006/007850). Another example is a recombinant polyclonal antibody against orthopoxvirus (WO 2007/065433). The use of site-specific integration results in a cell population where each cell contains one single copy and where expression levels and growth rates are expected to be relatively uniform.

### SUMMARY OF THE INVENTION

The present invention provides alternative methods for production of particular recombinant polyclonal anti-RSV antibodies, which methods are independent of site-specific integration and therefore provide increased flexibility with respect to the choice of cell line, while maintaining the polyclonality of the antibody. In addition, expression levels may be higher than possible with site-specific integration.

The approach of the present invention is based on random integration of the anti-RSV antibody encoding genes into host cells, preferably followed by cloning of stably transfected single cells with desired characteristics. The individual cell clones which each produce an individual member of the polyclonal anti-RSV antibody are then mixed in order to generate a polyclonal manufacturing cell line for the production of a polyclonal anti-RSV antibody.

Thus, in a first aspect the invention relates to a polyclonal cell line comprising 2 to n sub-populations of cells each sub-population expressing one distinct antibody member of a recombinant polyclonal anti-RSV antibody, the cells comprising at least one expression construct coding for one distinct antibody member randomly and stably integrated into the genome, wherein the distinct members of said recombinant polyclonal anti-RSV antibodies are selected from the group consisting of antibody molecules comprising CDR1, CDR2, and CDR3 regions selected from the group of the VH and VL pairs given in Table 3 herein. The invention also relates to methods for manufacturing recombinant polyclonal anti-RSV antibody comprising culturing such polyclonal cell line and recovering the polyclonal antibody from the supernatant.

The antibodies of Table 3 are fully human antibodies isolated from healthy donors that have been exposed to RSV infection and have raised an immune response against RSV. Therefore polyclonal antibodies comprising different antibodies from Table 3 reflect the human polyclonal immune response to RSV.

The present invention allows for the commercial production of a recombinant polyclonal anti-RSV antibody in one container, e.g. for use in pharmaceutical compositions. One important feature of the invention is that during the manufacturing process biased expression of the individual molecules constituting the polyclonal anti-RSV antibody is kept to a low level, minimizing unwanted batch-to-batch variation and avoiding elimination of members of the polyclonal anti-RSV antibody during manufacture.

In separate aspects the present invention relates to cell lines and methods for manufacturing particular monoclonal anti-RSV antibodies using expression systems relying on random integration of the expression constructs into the genome of the host cells.

Particularly, the invention relates to a cell comprising an expression construct capable of directing the expression of an anti-RSV antibody selected from the group consisting of antibodies comprising at least the complementarity-determining-regions (CDRs) of the antibodies listed in Table 3, wherein the cell comprises at least one expression construct stably integrated at a random position in the genome.

Such cells may be generated by transfecting cells with an expression construct coding for said anti-RSV antibody under conditions allowing random integration into the genome of said cell, and selecting at least one cell with an expression construct integrated stably at a random position, the expression construct coding for an anti-RSV antibody being selected from the group consisting of antibodies comprising at least the complementarity-determining-regions (CDRs) of the antibodies listed in Table 3.

In preferred embodiments the polyclonal cell line is used as a polyclonal manufacturing cell line and frozen and stored and used as a polyclonal Master Cell Bank (pMCB), from which samples can be thawed and used for a polyclonal Working Cell Bank (pWCB). For manufacturing of monoclonal antibody, a monoclonal cell line is used to generate a Master Cell Bank (MCB) from which a Working Cell Bank (WCB) may be generated.

### Definitions

By “protein” or “polypeptide” is meant any chain of amino acids, regardless of length or post-translational modification. Proteins can exist as monomers or multimers, comprising two or more assembled polypeptide chains, fragments of proteins, polypeptides, oligopeptides, or peptides.

The terms “a distinct member of a recombinant polyclonal antibody” denotes one antibody molecule of an antibody composition comprising different antibody molecules, where each antibody molecule is homologous to the other molecules of the composition, but also contains one or more stretches of variable polypeptide sequence, which is/are characterized by differences in the amino acid sequence between the individual members of the polyclonal antibody.

The term “antibody” describes a functional component of serum and is often referred to either as a collection of molecules (antibodies or immunoglobulins) or as one molecule (the antibody molecule or immunoglobulin molecule). An antibody molecule is capable of binding to or reacting with a specific antigenic determinant (the antigen or the antigenic epitope), which in turn may lead to induction of immunological effector mechanisms. An individual antibody molecule is usually regarded as monospecific, and a composition of antibody molecules may be monoclonal (i.e., consisting of identical antibody molecules) or polyclonal (i.e., consisting of different antibody molecules reacting with the same or different epitopes on the same antigen or even on distinct, different antigens). Each antibody molecule has a unique structure that enables it to bind specifically to its corresponding antigen, and all natural antibody molecules have the same overall basic structure of two identical light chains and two identical heavy chains. Antibodies are also known collectively as immunoglobulins. The terms antibody or antibodies as used herein are also intended to include chimeric and single chain antibodies, as well as binding fragments of antibodies, such as Fab, Fv fragments or scFv fragments, as well as multimeric forms such as dimeric IgA molecules or pentavalent IgM.

The term “polyclonal antibody” describes a composition of different antibody molecules which is capable of binding to or reacting with several different specific antigenic determinants on the same or on different antigens. Usually, the variability of a polyclonal antibody is thought to be located in the so-called variable regions of the polyclonal antibody. However, in the context of the present invention, polyclonality can also be understood to describe differences between the individual antibody molecules residing in so-called constant regions, e.g., as in the case of mixtures of antibodies containing two or more antibody isotypes such as the human isotypes IgG1, IgG2, IgG3, IgG4, IgAl, IgA2, IgM, IgD, and IgE, or the murine isotypes IgG1, IgG2a, IgG2b, IgG3, and IgA.

The term “immunoglobulin” commonly is used as a collective designation of the mixture of antibodies found in blood or serum, but may also be used to designate a mixture of antibodies derived from other sources.

The term “immunoglobulin molecule” denotes an individual antibody molecule, e.g., as being a part of immunoglobulin, or part of any polyclonal or monoclonal antibody composition.

The term “a library of variant nucleic acid molecules” is used to describe the collection of nucleic acid molecules, which collectively encode a “recombinant polyclonal anti-RSV-antibody”. When used for transfection, the library of variant nucleic acid molecules is contained in a library of expression vectors. Such a library typically have at least 3, 5, 10, 20, 50, 1000, 10⁴, 10⁵ or 10⁶ distinct members.

As used herein the term “distinct nucleic acid sequence” is to be understood as a nucleic acid sequence which may encode different polypeptide chains that together constitute the anti-RSV antibody. Where the distinct nucleic acid sequence is comprised of more than one encoding sequence, these sequences may be in the form of a dicistronic transcription unit or they may be operated as two separate transcriptional units if operably linked to suitable promoters. Likewise the use of tri- and quattrocistronic transcription units is conceivable if a selection marker is included into a transcriptional unit together with a nucleic acid coding for an antibody or a sub-unit thereof Preferably, a distinct nucleic acid sequence of the present invention is part of a nucleic acid molecule such as e.g. a vector. When introduced into the cell, the genes, which together encode the fully assembled antibody, reside in the same vector, thus being linked together in one nucleic acid sequence.

As used herein, the term “vector” refers to a nucleic acid molecule into which a nucleic acid sequence can be inserted for transport between different genetic environments and/or for expression in a host cell. If the vector carries regulatory elements for transcription of the nucleic acid sequence inserted in the vector (at least a suitable promoter), the vector is herein called “an expression vector”. In the present specification, “phagemid vector” and “phage vector” are used interchangeably. The terms “plasmid” and “vector” are used interchangeably. The invention is intended to include such other forms of vectors, which serve equivalent functions for example plasmids, phagemids and virus genomes or any nucleic acid molecules capable of directing the production of a desired protein in a proper host.

The term “each member of the library of vectors” is used to describe individual vector molecules with a distinct nucleic acid sequence derived from a library of vectors, where the nucleic acid sequence encodes one member of the recombinant polyclonal antibody.

The term “transfection” is herein used as a broad term for introducing foreign DNA into a cell. The term is also meant to cover other functional equivalent methods for introducing foreign DNA into a cell, such as e.g., transformation, infection, transduction or fusion of a donor cell and an acceptor cell.

The term “selection” is used to describe a method where cells have acquired a certain characteristic that enable the isolation from cells that have not acquired that characteristic. Such characteristics can be resistance to a cytotoxic agent or production of an essential nutrient, enzyme, or color.

The terms “selectable marker gene”, “selection marker gene”, “selection gene” and “marker gene” are used to describe a gene encoding a selectable marker (e.g., a gene conferring resistance against some cytotoxic drug such as certain antibiotics, a gene capable of producing an essential nutrient which can be depleted from the growth medium, a gene encoding an enzyme producing analyzable metabolites or a gene encoding a colored protein which for example can be sorted by FACS) which is co-introduced into the cells together with the gene(s) coding for the anti-RSV antibody.

The term “recombinant protein” is used to describe a protein that is expressed from a cell line transfected with an expression vector comprising the coding sequence of the protein.

As used herein, the term “operably linked” refers to a segment being linked to another segment when placed into a functional relationship with the other segment. For example, DNA encoding a signal sequence is operably linked to DNA encoding a polypeptide if it is expressed as a leader that participates in the transfer of the polypeptide to the endoplasmic reticulum. Also, a promoter or enhancer is operably linked to a coding sequence if it stimulates the transcription of the sequence.

The term “promoter” refers to a region of DNA involved in binding the RNA polymerase to initiate transcription.

The term “head-to-head promoters” refers to a promoter pair being placed in close proximity so that transcription of two gene fragments driven by the promoters occurs in opposite directions. A head-to-head promoter can also be constructed with a stuffer composed of irrelevant nucleic acids between the two promoters. Such a stuffer fragment can easily contain more than 500 nucleotides.

An “antibiotic resistance gene” is a gene encoding a protein that can overcome the inhibitory or toxic effect that an antibiotic has on a cell ensuring the survival and continued proliferation of cells in the presence of the antibiotic.

The term “internal ribosome entry site” or “IRES” describes a structure different from the normal 5′ cap-structure on an mRNA. Both structures can be recognized by a ribosome to initiate scanning for an AUG codon to initiate translation. By using one promoter sequence and two initiating AUG's, a first and a second polypeptide sequence can be translated from a single mRNA. Thus, to enable co-translation of a first and a second polynucleotide sequence from a single bi-cistronic mRNA, the first and second polynucleotide sequence can be transcriptionally fused via a linker sequence including an IRES sequence that enables translation of the polynucleotide sequence downstream of the IRES sequence. In this case, a transcribed bi-cistronic RNA molecule will be translated from both the capped 5′ end and from the internal IRES sequence of the bi-cistronic RNA molecule to thereby produce both the first and the second polypeptide.

The term “inducible expression” is used to describe expression that requires interaction of an inducer molecule or the release of a co-repressor molecule and a regulatory protein for expression to take place.

The term “constitutive expression” refers to expression which is not usually inducible.

The term “scrambling” describes situations where two or more distinct members of a polyclonal protein comprised of two different polypeptide chains, e.g. from the immunoglobulin superfamily, is expressed from an individual cell. This situation may arise when the individual cell has integrated, into the genome, more than one pair of gene segments, where each pair of gene segments encode a distinct member of the polyclonal protein. In such situations unintended combinations of the polypeptide chains expressed from the gene segments can be made. These unintended combinations of polypeptide chains might not have any therapeutic effect.

The term “V_{H}-V_{L} chain scrambling” is an example of the scrambling defined above. In this example the V_{H} and V_{L} encoding gene segments constitute a pair of gene segments. The scrambling occurs when unintended combinations of V_{H} and V_{L} polypeptides are produced from a cell where two different V_{H} and V_{L} encoding gene segment pairs are integrated into the same cell. Such a scrambled antibody molecule is not likely to retain the original specificity, and thus might not have any therapeutic effect.

The term “recombinant polyclonal manufacturing cell line” refers to a population of protein expressing cells that are transfected with a library of variant nucleic acid sequences such that the individual cells, which together constitute the recombinant polyclonal manufacturing cell line, carry one or more copies of a distinct nucleic acid sequence, which encodes one member of the recombinant polyclonal anti-RSV antibody, and one or more copies are integrated into the genome of each cell. The cells constituting the recombinant polyclonal manufacturing cell line are selected for their ability to retain the integrated distinct nucleic acid sequence, for example by antibiotic selection. Cells which can constitute such a manufacturing cell line can be for example bacteria, fungi, eukaryotic cells, such as yeast, insect cells or mammalian cells, especially immortal mammalian cell lines such as CHO cells, COS cells, BHK cells, myeloma cells (e.g., Sp2/0 cells, NSO, YB2/0), NIH 3T3, and immortalized human cells, such as HeLa cells, HEK 293 cells, or PER.C6.

The term “bias” is used to denote the phenomenon during recombinant polyclonal protein production, wherein the composition of a polyclonal vector, polyclonal cell line, or polyclonal protein alters over time due to random genetic mutations, differences in proliferation kinetics between individual cells, differences in expression levels between different expression construct sequences, or differences in the cloning efficiency of DNA.

The term “RFLP” refers to “restriction fragment length polymorphism”, a method whereby the migratory gel pattern of nucleic acid molecule fragments is analyzed after cleavage with restriction enzymes.

The term “5′ UTR” refers to a 5′ untranslated region of the mRNA.

The term “conditions avoiding site specific integration” refers to a transfection process which does not include any of the possible ways to obtain site specific integration. Site specific integration can e.g. be achieved using a combination of a recombinase and a recognition site for the recombinase in a chromosome of the host cell. The recombinase may also be covalently linked to a nucleotide stretch recognising a particular site in a chromosome. Site-specific integration can also be achieved - albeit at a lower efficiency—using homologous recombination. Avoiding site-specific integration will often result in integration at random positions throughout the genome of the host cell, if integration vectors are used.

The term “random integration” refers to integration of an expression vector into the genome of a host cell at positions that are random. The dictionary meaning of random is that there are equal chances for each item, in this case integration site. When transfecting cells all integration sites do not represent absolutely equal chances of integration as some parts of the chromosomes are more prone to integration events than others. When nothing is done to guide the expression vector to a particular integration site, it will integrate at positions that are random within the group of possible integration sites. Therefore, “random integration” in the context of the present invention is to be understood as a transfection procedure where nothing is done to guide the expression construct to a predetermined position. The absence of means to guide the expression vector to a predetermined position suffices to ensure “random integration”. Thereby integration site(s) will vary from cell to cell in a transfected population, and the exact integration site(s) can be regarded unpredictable.

The term “stably integrated” refers to integration of an expression vector into the genome of a host cell, wherein the integration remains stable over at least 20, more preferably 30, more preferably 40, more preferably 50, such as 75, for example 100 generations or more.

Abbreviations: “CMV”=(human) Cytomegalo Virus. “AdMLP”=Adenovirus Major Late Promoter. SV40 poly A=Simian Virus 40 poly A signal sequence. GFP=Green Flourescent Proteins. TcR=T cell receptor. ELISA=Enzyme-Linked Immunosorbent Assay. LTR=Long Terminal Repeat.

### DESCRIPTION OF THE DRAWINGS

FIG. 1. Schematic overview of the process for generating a polyclonal cell bank.

The figure schematically illustrates the steps required to obtain a polyclonal cell bank, e.g. a polyclonal master cell bank. a) illustrates different expression vectors Ab.₁, Ab.₂, Ab.₃, etc each encoding a different and distinct member of the polyclonal anti-RSV antibody. b) illustrates the host cells to be transfected with the expression vectors. c) illustrates integration of the expression vectors at different positions and in different copy numbers in individual cells. d) illustrates selection of cellular clones for each of the members of the polyclonal anti-RSV antibody. In this particular case, for ease of illustration, only one clone per distinct member of the polyclonal anti-RSV antibody is shown. Step e) illustrates mixing of the clones selected in step d) to generate a polyclonal cell bank.

FIG. 2a. Prototype vector encoding heavy and light chain. The elements are as follows:

two identical head-to-head human CMV promoters with a spacer element (stuffer) in between

coding regions for heavy (VH+IgG1 constant region) and light chain (kappa) bGH polyA=bovine growth hormone polyadenylation sequence SV40 polyA=SV40 polyadenylation sequence

Genomic leaders for heavy and light chain

IRES+DHFR=ECMV internal ribosome entry site and the mouse dihydrofolate reductase cDNA

pUC ori=pUC origin of replication

bla, amp=ampicilline resistance gene

FIG. 2b. E1A expression vector pML29. The vector is based on pcDNA3.1+(Invitrogen)

The elements are as follows:

CMV=human CMV promoter

E1a=cDNA for adenovirus type 5 13 S transactivator

bGH polyA=bovine growth hormone polyadenylation region

SV40EP=SV40 early promoter

Neo=the neo resistance gene

SV40 polyA=SV40 polyadenylation region

AMP=β-lactamase gene encoding ampicillin resistance

FIG. 3. SDS-PAGE under reducing (lanes 2-5) and non-reducing conditions (lanes 8 -11) of purified Sym003 antibodies 818-4 (lanes 2 and 8), 810-7 (lanes 3 and 9), 824-7 (lanes 4 and 10), and 824-18 (lanes 5 and 11). 1-8 μg purified protein was applied onto the gel. The suffixes (-4, -7, -7, -18) denote cellular clones expressing the antibodies.

### DETAILED DESCRIPTION OF THE INVENTION

The recombinant polyclonal anti-RSV antibody expression system

The present invention provides methods for the consistent manufacturing of recombinant polyclonal anti-RSV antibody. Such antibodies include complete antibodies, Fab fragments, Fv fragments, and single chain Fv (scFv) fragments. In particular, it is contemplated that the present invention can be used for large-scale manufacturing and production of recombinant therapeutic polyclonal anti-RSV antibodies.

One of the major advantages of the manufacturing method of the present invention is that all the members constituting the recombinant polyclonal anti-RSV antibody can be produced in one or a few bioreactors or equivalents thereof. Further, the recombinant polyclonal anti-RSV antibody composition can be purified from the reactor as a single preparation without having to separate the individual members constituting the recombinant polyclonal anti-RSV antibody during the process. The technology as described herein generally can produce a polyclonal anti-RSV antibody with many individual members, in principle without an upper limit.

The host cell line used is preferably a mammalian cell line comprising those typically used for biopharmaceutical protein expression, e.g., CHO cells, COS cells, BHK cells, myeloma cells (e.g., Sp2/0 cells, NSO, YB2/0), NIH 3T3, and immortalized human cells, such as HeLa cells, HEK 293 cells, or PER.C6. In the present invention CHO cells were used, more particularly a modified DG44 clone. The choice of this particular cell line has been made because CHO cells are widely used for recombinant manufacture of antibodies and because the DG44 clone can be used in combination with the metabolic selection marker DHFR, which additionally allows for amplification of the encoded gene. The DG44 cell line has been modified by tranfection and subcloned. This has been done to increase the overall yield. The sub-cloned cell line is a very stable cell line providing cell clones having uniform growth rates and uniform and high expression levels for different anti-RSV antibodies.

BHK-21 cells or dhfr-minus mutants of CHO such as CHO-DUKX-B11 or DG44 or CHO-S or CHO-K1, are preferred mammalian cells for the practice of this invention. These cells are well known in the art and widely available, for example, from the American Type Culture Collection, (A.T.C.C.) Rockville, Md. (BHK-21) or from Dr. Lawrence Chasin, Columbia University, New York (CHO DUKX-B11 or DG44). These cells adapt well to growth in suspension cultures and/or can grow under low serum concentrations and can be used in conjunction with the DHFR selection marker.

Consequently, a person of ordinary skill in the art would be able to substitute the DG44 clone with other clones and substitute CHO cells with other mammalian cells as described, or even utilize other types of cells, including plant cells, yeast cells, insect cells, fungi and bacteria. Thus the choice of cell type is not intended to be limiting to the invention.

The recombinant polyclonal anti-RSV antibody of the present invention is intended to cover a anti-RSV antibody composition comprising different, but homologous anti-RSV antibody molecules, which are naturally variable, meaning that, in preferred embodiments, the anti-RSV antibody comprises a naturally occurring diversity.

In the broadest aspect the polyclonal cell line comprises 2 to n sub-populations of cells each sub-population expressing one distinct antibody member of a recombinant polyclonal anti-RSV antibody, the cells comprising at least one expression construct coding for one distinct antibody member randomly and stably integrated into the genome, wherein the distinct members of said recombinant polyclonal anti-RSV antibodies are selected from the group consisting of antibody molecules comprising CDR1, CDR2, and CDR3 regions selected from the group of the VH and VL pairs given in Table 3 herein.

The antibodies with the CDR sequences of Table 3 were isolated from healthy adults that have been exposed to RSV infection. Therefore the antibodies reflect the natural human immune response to RSV infection and combinations of antibodies based on these specific antibodies can be made to mirror the natural human immune response.

Preferred combinations of antibodies from Table 3 are constituted by the antibody compositions 1 to 56 in Table 6 herein. All of the antibody combinations of Table 6 herein have been tested for in vitro neutralization against one or more RSV strains and many are very potent.

Particularly preferred are antibody combinations wherein the distinct members are combined as in any one of the antibody compositions 2, 9, 13, 17, 18, 28, 33, and 56 in Table 6 herein, even more preferably any one of the antibody compositions 28, 33, and 56. These combinations are very potent, and have been tested in an animal model of RSV infection. They are are capable of reducing lung virus load significantly when administered prophylactically.

In other embodiments the distinct members of the polyclonal antibody are selected from the group consisting of antibodies comprising the VH and VL sequences of clones 735, 736, 744, 793, 795, 796, 799, 800, 801, 804, 810, 811, 812, 814, 816, 817, 818, 819, 824, 825, 827, 829, 830, 831, 835, 838, 841, 853, 855, 856, 857, 858, 859, 861, 863, 868, 870, 871, 880, 881, 884, 886, 888, and 894 as defined herein.

In preferred embodiments, the distinct members are selected from the group consisting of antibodies from clones 793, 800, 810, 816, 818, 819, 824, 825, 827, 831, 853, 855, 856, 858, 868, 880, 888, and 894, and antibodies including the CDRs of said antibodies. These antibodies have been tested as monoclonal antibodies in virus neutralisation assays against one or more RSV isolates (Table 5).

Modified CHO cells comprising randomly integrated expression constructs have been prepared for expression of the following antibodies comprising the VH and VL sequences of clones 810, 818, 819, 824, 825, 827, 858, 894, 793, 816, 853, 855, and 856. These antibodies have been tested in several combinations in Table 6 and are preferred antibodies for making a polyclonal anti-RSV antibody.

Particularly preferred antibodies for inclusion into a polyclonal anti-RSV antibody are the antibody encoded by clone 824 or an antibody with the CDRs of clone 824; and the antibody encoded by clone 810 or an antibody with the CDRs of clone 810.

In order to obtain a potent anti-RSV antibody it is preferable that the polyclonal anti-RSV antibody comprises at least one distinct antibody molecule capable of binding the F protein, and at least one distinct antibody molecule capable of binding the G-protein. More preferably it includes at least two antibodies targeting the F-protein and two antibodies targeting the G-protein. Even more preferably, the composition comprises at least 3 antibodies against each of the two target proteins, F and G.

The polyclonal antibody may comprise 2 or more antibodies, such as preferably 3 or more, for example 4 or more, such as 5 or more, for example 6 or more, such as 7 or more, for example 8 or more, such as 9 or more, for example 10 or more, such as 15 or more, for example 20 or more, such as 25 or more, for example 30 or more, such as 40 or more, for example 50 or more.

As the number of distinct antibody molecules in the polyclonal antibody increases the concentration of each antibody in the final product is reduced assuming that an equal amount of each antibody is present. Furthermore, with increasing numbers of antibodies expressed by a polyclonal cell line, the risk that one of the antibodies is lost during manufacture increases. Therefore, the polyclonal antibody preferably comprises less than 50 antibodies, such as less than 40 antibodies, for example less than 30 antibodies, such as less than 25 antibodies, for example less than 20 antibodies or even less than 15 antibodies.

In the context of the present invention, variability in the polypeptide sequence (the polyclonality) can also be understood to describe differences between the individual antibody molecules residing in so-called constant regions or C regions of the antibody polypeptide chains, e.g., as in the case of mixtures of antibodies containing two or more different antibody isotypes, such as the human isotypes IgG1, IgG2, IgG3, IgG4, IgA1, IgA2, IgM, IgD, and IgE. Thus, a recombinant polyclonal anti-RSV antibody may comprise antibody molecules that are characterized by sequence differences between the individual antibody molecules in the variable region (V region) or in the constant region (C region) or both. Preferably, the antibodies are of the same isotype, as this eases the subsequent purification considerably. It is also conceivable to combine antibodies of e.g. isotype IgG1, IgG2, and IgG4, as these can all be purified together using Protein A affinity chromatography. In a preferred embodiment, all antibodies constituting the polyclonal antibody have the same constant region to further facilitate purification. More preferably, the antibodies have the same constant region of the heavy chain. The constant region of the light chain may also be the same across distinct antibodies.

Polyclonality in the so-called constant region, particularly the heavy chain of the antibodies, is of interest with regard to therapeutic application of antibodies. The various immunoglobulin isotypes have different biological functions (summarized in Table 1), which might be desirable to combine when utilizing antibodies for treatment because different isotypes of immunoglobulin may be implicated in different aspects of natural immune responses (Canfield and Morrison 1991. J.Exp.Med. 173, 1483-91; Kumpel et al. 2002. Transfus.Clin.Biol. 9, 45.-53; Stirnadel et al. 2000. Epidemiol. Infect.124, 153-162).

TABLE 1
Biological functions of the human immunoglobulin isotypes

| | Human Immunoglobulin | | | | | | | | |
|---|---|---|---|---|---|---|---|---|---|
| | IgG₁ | IgG₂ | IgG₃ | IgG₄ | IgA₁ | IgA₂ | IgM | IgD | IgE |
| Classical comple- | +++ | ++ | ++++ | + | − | − | ++++ | − | − |
| ment activation | | | | | | | | | |
| Alternate comple- | + | + | + | +++ | + | − | − | + | − |
| ment activation | | | | | | | | | |
| Placental transfer | + | ++ | + | ++ | − | − | − | − | − |
| Bacterial lysis | + | + | + | + | +++ | +++ | + | ? | ? |
| Macrophage/other | + | − | + | + | + | + | − | − | − |
| phagocytes binding | | | | | | | | | |
| Mast cell/basophils | − | − | − | − | − | − | − | − | − |
| binding | | | | | | | | | |
| Staphylococcal Pro- | + | + | − | + | − | − | − | − | − |
| tein A reactivity | | | | | | | | | |

### Clonal Diversity

Clonal diversity of the cell line may be analyzed by RFLP on isolated clones from a pool of cells expressing a recombinant polyclonal protein. Sequencing of (RT)-PCR products represents another possibility to analyse clonal diversity. The diversity can also be analyzed by functional tests (e.g., ELISA) on the recombinant polyclonal anti-RSV antibody produced by the cell line. WO 2006/007853 discloses methods for characterization of a polyclonal cell line and a polyclonal protein. These methods can be used for analyzing the clonal diversity of the cell line and the resulting polyclonal anti-RSV antibody.

Clonal bias (i.e., a gradual change in the content of the individual antibodies constituting the polyclonal antibody), if it exists, can be estimated by comparing the clonal diversity of the initial library, used for transfection, with the diversity found in the pool of cells (cell line) expressing the recombinant polyclonal anti-RSV antibody.

Clonal diversity may be assessed as the distribution of individual members of the polyclonal anti-RSV antibody. This distribution can be assessed as the total number of different individual members in the final polyclonal anti-RSV antibody composition compared to the number of different encoding sequences originally introduced into the cell line during transfection. In this case sufficient diversity is considered to be acquired when at least 50% of the encoding sequences originally used in the transfection can be identified as different individual members of the final polyclonal anti-RSV antibody, preferably at least 75%, more preferably at least 80%, more preferably at least 90%, such as at least 95%, 97%, 98% or 99%. Expressed in another way, clonal diversity can be considered sufficient if only 1 member of the polyclonal anti-RSV antibody is lost during manufacture, of if 2, 3, 4 or 5 members are lost.

Preferably, the distribution of individual members of the polyclonal composition is assessed with respect to the mutual distribution among the individual members. In this case sufficient clonal diversity is considered to be acquired if no single member of the anti-RSV antibody composition constitutes more than 75% of the total amount of protein in the final polyclonal anti-RSV antibody composition. Preferably, no individual member exceeds more than 50%, even more preferred 25% and most preferred 10% of the total amount of antibody in the final polyclonal anti-RSV antibody composition. The assessment of clonal diversity based on the distribution of the individual members in the polyclonal anti-RSV antibody composition can be performed by RFLP analysis, sequence analysis and protein analysis such as the approaches described later on for characterization of a polyclonal anti-RSV antibody.

Clonal diversity may also be defined by setting a predefined relative amount of each antibody in the fmal product. For a polyclonal antibody with 10 distinct antibodies, the predefined relative amount may be e.g. 10% for each antibody. The predefined relative amount may also be different for each distinct antibody. Clonal diversity can then be said to be sufficient if the amount of a distinct antibody in the produce differs less than 75% from the predefined relative amount. Preferably less than 50%, even more preferred less than 25%, and most preferred less than 10% from the predefined relative amount.

Clonal diversity may be reduced as a result of clonal bias which can arise a) as a result of differences in expression level, b) as a result of variations in cellular proliferation. If such biases arise, each of these sources of a loss of clonal diversity may be remedied by minor modifications to the methods as described herein.

It is possible that variations in cellular proliferation rates of the individual cells in the cell line could, over a prolonged period of time, introduce a bias into the recombinant polyclonal anti-RSV antibody expression, increasing or reducing the presence of some members of the recombinant polyclonal protein expressed by the cell line. As the present methods are based on random integration into the genome of the host cell, both the position and the copy number vary between members of the polyclonal cell line. This may give rise to differences in proliferation rate and expression level among clones. By selecting cellular clones with similar proliferation rate this problem is minimized. A further possibility is to use more than one clone for each member of the polyclonal protein. The compositional stability may be increased if e.g. between 3- and 5 clones expressing a single member of the polyclonal protein is used compared to only one clone for each member of the polyclonal anti-RSV antibody.

Cells expressing one distinct member of the recombinant polyclonal protein is preferably derived from 1 or more cloned cells, such as from 2 or more, for example from 3 or more, such as from 4 or more, for example from 5 or more, such as from 6 or more, for example from 7 or more, such as from 8 or more, for example from 9 or more, such as from 10 or more for example 11 or more, such as 12 or more, for example 13 or more, such as 14 or more, for example 15 or more, such as 16 or more, for example 17 or more, such as 18 or more, for example 19 or more, such as 20 or more, for example 21 or more, such as 22 or more, for example 23 or more, such as 24 or more, for example 25 or more, such as 26 or more, for example 27 or more, such as 28 or more, for example 29 or more, such as 30 or more, for example 35 or more, such as 40 or more, for example 45 or more, such as 50 or more, for example 60 or more, such as 70 or more, for example 80 or more, such as 90 or more, for example 100 or more. For most purposes the number of cloned cells is less than 50, for example less than 20, such as less than 15, for example less than 10.

Another way to address this issue is to use one or more selection criteria to ensure that the cells are uniform within certain pre-set limits with respect to one or more criteria selected from the group consisting of growth rate, doubling time, expression level, production level, stability of production over time, viability, hardiness, robustness, morphology, and copy number.

One reason for variations in proliferation rates could be that the population of cells constituting the starting cell line used for the initial transfection is heterogeneous. It is known that individual cells in a cell line develop differently over a prolonged period of time. To ensure a more homogeneous starting material, sub-cloning or repeated sub-cloning of the cell line prior to transfection with the expression vectors may be performed using a limiting dilution of the cell line down to the single cell level and growing each single cell to a new population of cells (so-called cellular sub-cloning by limiting dilution).

An alternative and preferred method for single cell cloning to ensure a well defined cell population is to use fluorescence activated cell sorting (FACS) after the transfection but prior to the selection procedure. Fluorescence labeled antibodies can be used to enrich for highly productive cells derived from a pool of cells transfected with IgG constructs (Brezinsky et al. J. 2003. Immunol Methods 277, 141-155). The advantage of using FACS sorting is that the method combines single cell cloning (by sorting single cells into wells), while simultaneously providing information about the expression level of each single cell. To further improve the sorting procedure, a viability stain can be included so that dead or dying cells are discarded. The FACS procedure subjects cells to rather severe conditions including shear stress. This means that indirectly cells are selected for resistance to such conditions. Furthermore, the FACS procedure is automated allowing for sorting of a high number of single cells.

The FACS method can also be used to sort cells expressing similar levels of immunoglobulin, thereby creating a homogenous cell population with respect to productivity. Likewise, by using labeling with the fluorescent dye 5,6-carboxylfluorescein diacetate succinimidyl ester (CFSE) cells showing similar proliferation rates can be selected by FACS methods.

An important embodiment of the present invention is the generation of one or more cloned cell lines for each member of the polyclonal anti-RSV antibody. The generation of single cell clones may be carried out using any one of a number of standard techniques. However, it has turned out that FACS cell sorting where cells are selected for viability and IgG levels and are sorted individually into wells has consistently turned out to provide stable clones suitable for preparing a polyclonal working cell bank. Individual clones are preferably selected after a certain number of days in culture under selection pressure following the cell sorting. As clones .are selected on the same day following sorting, the growth rate of the clones will be relatively uniform. In addition to this, colonies are inspected visually to discard clones with gross changes in morphology and low growth rates compared to the original untransfected cell line. Finally, the level of antibody expression can be assayed using e.g. ELISA or other analytical techniques and clones with high and relatively uniform expression levels can be selected.

Even if a proliferation rate-induced bias does develop, the loss or over-representation of individual members may not necessarily be critical, depending on the diversity requirements of the final recombinant polyclonal protein product and the stability of the diversity over time. Recombinant monoclonal anti-RSV manufacturing system

The invention also relates to cell lines for expression of certain monoclonal anti-RSV antibodies. Much of what is stated about establishment of cell lines, selection of cells, design of vectors, cloning strategies, culturing of cells, and recovery of antibody for polyclonal antibodies also relates to the monoclonal aspects of the invention.

In the broadest “monoclonal” aspect the invention relates to a cell comprising an expression construct capable of directing the expression of an anti-RSV antibody selected from the group consisting of antibodies comprising at least the complementarity-determining-regions (CDRs) of the antibodies listed in Table 3, wherein the cell comprises at least one expression construct stably integrated at a random position in the genome.

Such cells may be generated by transfecting a cell with an expression construct coding for said anti-RSV antibody as defined above under conditions allowing random integration into the genome of said cell, and selecting at least one cell with an expression construct integrated stably at a random position. Transfection under such conditions often leads to integration of two or more expression constructs at random positions into the genome of the host cell.

In order to make full use of the random integration transfection and/or selection may be carried out under conditions favouring amplification of the expression construct and resulting in even higher expression levels.

In certain embodiments, the monoclonal anti-RSV antibody is selected from the group consisting of antibodies which include the CDRs from the V_{H} and V_{L} sequence pairs of clones 735, 736, 744, 793, 795, 796, 799, 800, 801, 804, 810, 811, 812, 814, 816, 817, 818, 819, 824, 825, 827, 829, 830, 831, 835, 838, 841, 853, 855, 856, 857, 858, 859, 861, 863, 868, 870, 871, 880, 881, 884, 886, 888, and 894. The V_{H} and light chain sequences for these clones are given herein.

In preferred embodiments the monoclonal anti-RSV antibody is selected from the group consisting of antibodies from clones 793, 800, 810, 816, 818, 819, 824, 825, 827, 831, 853, 855, 856, 858, 868, 880, 888, and 894, and antibodies including the CDRs of said antibodies.

Even more preferred, the monoclonal anti-RSV antibody is selected from the group consisting of antibodies from clones 793, 800, 810, 818, 819, 824, 825, 827, 831, 853, 858, 888, and 894.

Particularly preferred are monoclonal antibodies wherein the CDRs are from clone 810 and even more preferred monoclonal antibodies, wherein the CDRs are from clone 824. Both antibodies have shown superior virus neutralisation potency and have are also superior when tested in an animal model of RSV infection.

### The Host Cell

Host cells can be generated from any cell which can integrate DNA into their chromosomes or retain extra-chromosomal elements such as mini-chromosomes, YACs (Yeast artificial chromosomes), MACs (Mouse artificial chromosomes), or HACs (Human artificial chromosomes). MACs and HACs are described in detail in WO 97/40183, hereby incorporated by reference. Preferably mammalian cells such as CHO cells, COS cells, BHK cells, myeloma cells (e.g., Sp2/0, YB2/0 or NSO cells), fibroblasts such as NIH 3T3, and immortalized human cells, such as HeLa cells, HEK 293 cells, or PER.C6, are used. However, non-mammalian eukaryotic or prokaryotic cells, such as plant cells, insect cells, yeast cells, fungi, E. coli etc., can also be employed. The same host cells can be used for mono- and polyclonal antibody expression.

In one embodiment of the present invention, the cell line which is to be used as starting material is sub-cloned by performing a so-called limiting dilution of the cell line down to a single cell level, followed by growing each single cell to a new population of cells prior to transfection with the library of vectors. Such sub-cloning can also be performed later in the process of selecting the right cell line, if desired. Other methods for single cell cloning include: FACS cloning (Brezinsky et al. J. 2003. Immunol Methods 277, 141-155), LEAP™ technology (from Cyntellect, San Diego, California, USA), and ClonePix (from Genetix, UK).

### The Vector for Integration

A suitable vector comprises a suitable selection gene. Suitable selection genes for use in mammalian cell expression include, but are not limited to, genes enabling for nutritional selection, such as the thymidine kinase gene (TK), glutamine synthetase gene (GS), tryptophan synthase gene (trpB) or histidinol dehydrogenese gene (hisD). Further, selection markers are antimetabolite resistance genes conferring drug resistance, such as the dihydrofolate reductase gene (dhfr) which can be selected for with hypoxanthine and thymidine deficient medium and further selected for with methotrexate, the xanthine-guanine phosphoribosyltransferase gene (gpt), which can be selected for with mycophenolic acid, the neomycin phosphotransferase gene (neo) which can be selected for with G418 in eukaryotic cell and neomycin or kanamycin in prokaryotic cells, the hygromycin B phosphotransferase (hyg, hph, hpt) gene which can be selected for with hygromycin, the puromycin N-acetyl-transferase gene (pac) which can be selected with puromycin or the Blasticidin S deaminase gene(Bsd) which can be selected with blasticidin, the Zeocin resistance gene (Sh ble) which mediates resistance towards Zeocin and Bleomycin. Finally, genes encoding proteins that enables sorting e.g. by flow cytometry can also be used as selection markers, such as green fluorescent protein (GFP), the nerve growth factor receptor (NGFR) or other membrane proteins, or beta-galactosidase (LacZ).

The selection marker may be located on a separate expression vector, thus performing co-transfection with an expression vector coding for the selection marker and one or more expression vector(s) coding for the anti-RSV antibody or subunits of an anti-RSV antibody. The selection marker may also be located in the expression vector coding for the antibody. In this latter case, the selection marker is preferably located on a transcript which also encodes the antibody or one of its sub-units. This can be done e.g. using an IRES construct. In the case of an antibody, the selection marker is preferably located on the transcript which encodes the largest sub-unit, such as for example the heavy chain of an antibody.

The vector for integration of the antibody gene further comprises DNA encoding one member of the recombinant polyclonal anti-RSV antibody, preceded by its own mammalian promoter directing expression of the protein. The DNA encoding the chains of the anti-RSV antibody can be preceded by their own mammalian promoter directing high levels of expression (bi-directional or uni-directional) of each of the chains. In a bi-directional expression a head-to-head promoter configuration in the expression vector can be used and for a uni-directional expression two promoters or one promoter combined with e.g., an IRES sequence can be used for expression. A bi-cistronic expression vector with two different subunits encoded by the same transcript and separated by an IRES sequence is likewise conceivable.

Suitable head-to-head promoter configurations are for example, but not limited to, the AdMLP promoter together with the mouse metallothionein-1 promoter in both orientations, the AdMLP promoter together with the elongation factor-1 promoter in both orientations or the CMV promoter together with the MPSV promoter in both orientations, or the CMV promoter used in both orientations.

In the case of antibodies, experience has shown that the amount of heavy chain expressed by a cell should not exceed the amount of light chain. Therefore, the promoter directing expression of the light chain is preferably at least as strong as the promoter directing expression of the heavy chain.

A nucleic acid sequence encoding a functional leader sequence can be included in the expression vector to direct the gene product to the endoplasmic reticulum or a specific location within the cell such as an organelle. A strong polyadenylation signal can be situated 3′ of the protein-encoding DNA sequence. The polyadenylation signal ensures termination and polyadenylation of the nascent RNA transcript and is correlated with message stability. The DNA encoding a member of the recombinant polyclonal anti-RSV antibody can, for example, encode both the heavy and light chains of an antibody or antibody fragments, each gene sequence optionally being preceded by their own mammalian promoter elements and/or followed by strong poly A signals directing high level expression of each of the two chains.

The expression vector for integration can carry additional transcriptional regulatory elements, such as enhancers, anti-repressors, or UCOE (ubiquitous chromatin opening elements) for increased expression at the site of integration. Enhancers are nucleic acid sequences that interact specifically with nuclear proteins involved in transcription. The UCOE opens chromatin or maintains chromatin in an open state and facilitates reproducible expression of an operably-linked gene (described in more detail in WO 00/05393 and Benton et al, Cytotechnology 38:43-46, 2002). Further enhancers include Matrix Attachment Regions (MARs) as described e.g. in Girod & Mermod 2003 (“Chapter 10: Use of scaffold/matrix-attachment regions for protein production”, pp 359-379 in Gene Transfer and Expression in Mammalian Cells, SC Makrides (ed), 2003, Elsevier Science BV). Anti-repressor elements include but are not limited to STAR elements (Kwaks et al Nat Biotechnol. 2003 May; 21(5):553-8). When one or more of the regulatory elements described in the above are integrated into the chromosome of a host cell they are termed heterologous regulatory elements. Establishing an expression system for high-level expression of anti-RSV antibody

Methods for introducing a nucleic acid sequence into a cell are known in the art. These methods typically include the use of a DNA vector to introduce the sequence of interest into the cell, the genome or an extra-chromosomal element. Transfection of cells may be accomplished by a number of methods known to those skilled in the art, including lipofection, chemically mediated transfection, calcium phosphate precipitation, electroporation, microinjection, liposome fusion, RBC ghost fusion, protoplast fusion, virus transduction, and the like.

For the transfection of a host cell line, a library of vectors, wherein each vector comprises only one copy of a nucleic acid sequence encoding one member of a recombinant polyclonal anti-RSV antibody, is used. This library of expression vectors collectively encodes the recombinant polyclonal anti-RSV antibody. Suitable vectors for integration were described in the previous section.

The generation of a recombinant polyclonal manufacturing cell line and the production of a recombinant polyclonal anti-RSV antibody from such a cell line can be obtained by several different transfection and manufacturing strategies.

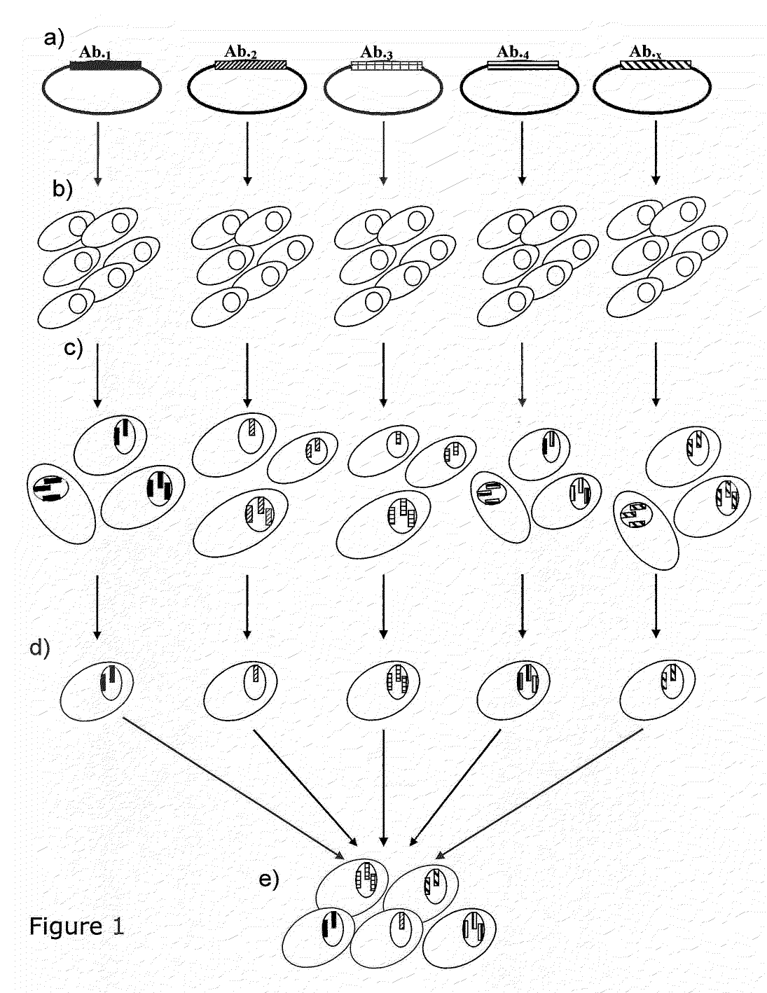

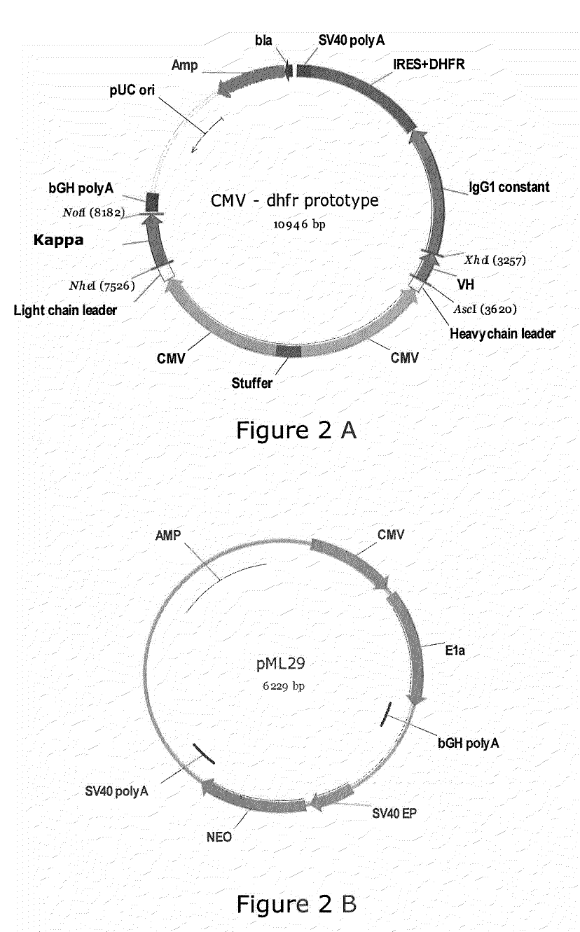

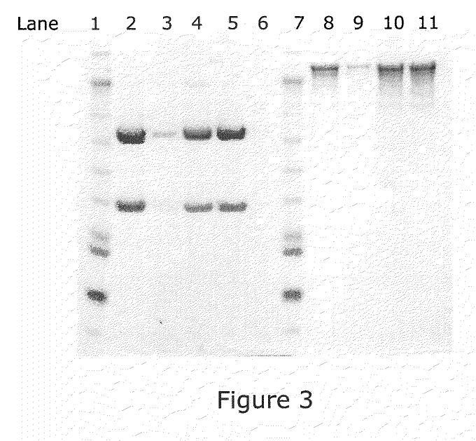

A preferred way of transfection illustrated in FIG. 1, is a high throughput method in which host cells are transfected separately using the individual vectors constituting the library. This method is termed individual transfection. The individually transfected host cells are preferably selected separately. However, they may also be pooled before selection. The individual cell clones generated upon selection may be analyzed with respect to expression level, proliferation rate and integration pattern and preferably, those with similar growth rates, similar copy number, similar expression and/or similar robustness levels may be used to generate a polyclonal anti-RSV antibody library stock. The individual cell clones can be mixed to obtain the desired polyclonal cell line before generating the stock, immediately after they have been retrieved from the stock, or after a short proliferation and adaptation time. This approach may further improve compositional stability. Steps a-d may be used to establish cell lines for expression of monoclonal anti-RSV antibody.

For anti-RSV antibody, bulk transfection allowing multiple integration into the genome of a host cell, would result in scrambling of the subunits. In many cases, such as the manufacture of recombinant polyclonal anti-RSV antibody for pharmaceutical use, scrambling is to be avoided. For multimeric proteins, bulk transfection can be done if scrambling is acceptable or if transfection is carried out under conditions ensuring integration of only one copy into the genome of each host cell. Examples of such methods include retroviral transduction and sphaeroblast fusion.

A frozen stock of the polyclonal cell line may be generated before initiation of the recombinant polyclonal anti-RSV antibody manufacturing. To obtain the desired polyclonal cell line for manufacturing, the clones can be mixed before generating the freezing stock, immediately after they have been retrieved from the stock or after a short proliferation and adaptation time.

A shared feature in the manufacturing strategies outlined in the above is that all the individual members constituting the recombinant polyclonal anti-RSV antibody can be produced in one, or a limited number of containers, such as bioreactors.

If expression levels need to be increased, gene amplification can be performed using selection for a DHFR gene or a glutamine synthetase (GS) gene, a hprt (hypoxanthin phosphoribosyltransferase) or a tryptophan synthetase gene. This requires the use of vectors comprising such a selection marker. One particular feature of the present invention is to keep the copy number relatively low in order to keep the stability of the cells high. Therefore, cells are preferably only subjected to one round of selection under relatively modest selection pressure (e.g. in nucleoside free medium with a low concentration of MTX (e.g. 1-10 nM) for the type of construct used in the examples). Modest selection pressure is believed to lead to a balanced copy number resulting in high expression while avoiding the instability of cells with very high copy number.

In order to achieve higher expression levels, the cell line used for expression may include a heterologous transactivator capable of enhancing the promoter controlling expression of the polyclonal anti-RSV antibody. Examples of suitable combinations of transactivator and promoter are listed below

| Transactivator | Promoter Examples |
|---|---|
| lentivirus Tat | long terminal repeat (LTR) |
| adenovirus E1A | HCMV major IE enhancer/promoter |
| herpes simplex virus VP16 | herpes simplex virus gene promoter |
| | is IE175 (U.S. Pat. No. 6,635,478) |
| hepatitis B virus X protein (HBx) | SV40early |
| Synthetic Zn-finger proteins | Synthetic |
| SV40 largeT antigen | SV40 late promoter |
| tetracycline-controlled | Synthetic |
| transactivators (tTA) | |
| Human cytomegalovirus IE2p86 | HCMV major IE enhancer/promoter |
| Human cytomegalovirus IE1p72 | HCMV major IE enhancer/promoter |
| Epstein-Barr virus R transactivator | EBV promoter |
| (Rta) | |
| thyroid hormone receptors | growth hormone promoter |
| glucocorticoid hormone receptors | mammary tumor virus (MMTV) |
| | promoter |

Preferably, the cell line is transfected with an expression construct coding for the transactivator and clones are selected using limiting dilution or other methods for single cell cloning. The expression vector may comprise elements such as promoter, selection marker etc as described for expression vectors herein. Preferably the promoter controlling expression of the transactivator is a constitutive promoter such as Elongation factor 1 promoter, CMV promoter, metallothionein-1 promoter or similar. In a preferred embodiment, the promoter is the CMV promoter.

For the manufacturing of a polyclonal anti-RSV antibody, where each anti-RSV antibody member is comprised of two polypeptide chains, the combination of the chains is of importance for the affinity, specificity and activity of the anti-RSV antibody they form. For this reason the polypeptide chains constituting an individual member of the polyclonal anti-RSV antibody are preferably placed in the same vector used for integration, thereby ensuring that they will be kept together throughout the process. Alternatively, the host cells can be transfected with pairs of expression vectors coding for cognate pairs of heavy and light chain.

The following description is one example of how to obtain a recombinant polyclonal anti-RSV antibody expressing cell line.

A universal promoter cassette for constitutive expression having two promoters placed in opposite transcriptional direction, such as a head-to-head construction surrounded by the variable heavy chain and the whole of the kappa or lambda light chain may be constructed, allowing transfer of the whole construct into a vector comprising a selection marker and the heavy chain constant region. It is contemplated that a promoter cassette for inducible expression can also be used. Furthermore, the promoters can be placed tail-to-tail which will result in transcription in opposite direction or tail-to-head for unidirectional transcription. An inducible promoter can also be used for control of the expression. After transfection, the cells are preferably cultivated under selective conditions to select stable tranformants.

Cells that survive under these conditions can subsequently be grown in different culture systems, such as conventional small culture flasks, Nunc multilayer cell factories, small high yield bioreactors (MiniPerm, INTEGRA-CELLine) and spinner flasks to hollow fiber-and bioreactors WAVE bags (Wave Biotech, Tagelswangen, Switzerland). The cells may be tested for antibody production using ELISA. Polyclonal cell lines are preferably selected for viability in suspension growth in serum free medium under selection pressure for extended periods. Evaluation of the preservation of polyclonality in the expression system

According to the present invention, it is often important to ensure that the polyclonality in the expression system is not seriously altered during production so that it is possible to stop the production when polyclonality is indeed altered. This is according to the invention done by monitoring the relative expression levels of the variant nucleic acid sequences. The expression levels can for example be monitored at mRNA level using for example RFLP analysis, arrays or real-time PCR, or at the protein level using for example two-dimensional polyacrylamide gel electrophoresis, mass spectrometry or various chromatographic techniques. With these techniques it will be possible to establish a baseline value for a number of all of the individual expression levels and then take out samples from the culture during production in order to gauge whether expression levels have changed (both in total and relatively). In normal practice of the invention, a range of values surrounding the baseline values can be established, and if the relative expression levels are found to be outside the ranges, then production is terminated.

### Cultivation of Cells and Production of a Recombinant Polyclonal Anti-RSV Antibody

The methods described herein apply also to the manufacture of monoclonal anti-RSV antibodies of the invention.

The polyclonal cell line produced as described above may be grown in suitable media under suitable conditions for expressing the polyclonal anti-RSV antibody encoded by the variant nucleic acid sequences inserted into the genome of the cells. The cell cultivation may be performed in several steps. When using mammalian cells, the selected cells are preferably adapted to growth in suspension as well as serum free conditions. Adaptation to growth in serum free medium may also advantageously be done before mixing the cloned cell lines for the polyclonal cell line. Adaptation can be performed in one or two steps and with or without selection pressure. Preferably, a selection system is used which allows for selection throughout the manufacturing period without compromising the purity of the manufactured drug product. In general, for manufacture of recombinant anti-RSV antibody for pharmaceutical use it is preferred not to use e.g. antibiotics or other low molecular weight drugs to provide selection pressure, as it will be needed to validate that the final product does not contain any traces of the antibiotic.

When the polyclonal cell line is adapted to the appropriate conditions scaling up can be initiated. At this point a polyclonal working cell stock (polyclonal working cell bank, pWCB) and/or polyclonal master cell bank (pMCB) can be frozen down. Preferably bioreactors of between 30 and 100 liters are used, but smaller (5-10 litres) or larger (up to 1,000, 5,000, 10,000, 15,000 liters, or even larger) bioreactors may be employed. The suitable production time and choice of bioreactor size are dependent on the desired yield of protein from the batch and expression levels from the cell line. Times may vary from a couple of days up to three months. The expressed recombinant polyclonal anti-RSV antibody may be recovered from the cells or the supernatant. The recombinant anti-RSV antibody may be purified and characterized according to procedures known by a person skilled in the art. Examples of purification procedures are listed below. Examples of characterization procedures can be found in e.g. WO 2006/007853.

Purification of a Recombinant Polyclonal Anti-RSV Antibody from Culture Supernatant

Isolation of anti-RSV antibody from culture supernatants is possible using various chromatographic techniques that utilize differences in the physico-chemical properties of proteins, e.g. differences in molecular weight, net charge, hydrophobicity, or affinity towards a specific ligand or protein. Proteins may thus be separated according to molecular weight using gel filtration chromatography or according to net charge using ion-exchange (cation/anion) chromatography or alternatively using chromatofocusing. Similarly, proteins may be separated according to hydrophobicity using hydrophobic interaction or charge induction chromatography or affinity chromatography utilizing differences in affinity towards a specific immobilized ligand or protein. Purification of complex mixtures of proteins such as an anti-RSV antibody, may thus be achieved by sequential combination of various chromatographic principles.

Affinity chromatography combined with subsequent purification steps such as ion-exchange chromatography, hydrophobic interactions and gel filtration has frequently been used for the purification of IgG (polyclonal as well as monoclonal) from e.g. cell culture supernatants. Affinity purification, where the separation is based on a reversible interaction between the protein(s) and a specific ligand coupled to a chromatographic matrix, is an easy and rapid method, which offers high selectivity, usually high capacity and concentration into a smaller volume. Protein A and protein G, two bacterial cell surface proteins, have high affinity for the Fc region, and have, in an immobilized form, been used for many routine applications, including purification of mono- and polyclonal IgG and its subclasses from various species and absorption and purification of immune complexes.

Following affinity chromatography, downstream chromatography, steps, e.g. ion-exchange and/or hydrophobic interaction chromatography, can be performed to remove host cell proteins, leaked Protein A, and DNA.

Gel filtration, as a final purification step, can be used to remove contaminant molecules such as dimers and other aggregates, and transfer the sample into storage buffer. Depending on the source and expression conditions it may be necessary to include an additional purification step to achieve the required level of antibody purity. Hydrophobic interaction chromatography or ion-exchange chromatography are thus frequently used, in combination with Protein A and gelfiltration chromatography, to purify antibodies for therapeutic use.

In order to ease the purification, it is preferable that all members of the polyclonal anti-RSV antibody share the same constant region of the heavy and/or light chain

In order to purify other classes of antibodies, alternative affinity chromatography media have to be used since proteins A and G do not bind IgA and IgM. An immunoaffinity purification can be used (anti-IgA or anti-IgM monoclonal antibodies coupled to solid phase) or, alternatively, multistep purification strategies including ion-exchange and hydrophobic interaction can be employed.

When purifying one of the monoclonal antibodies disclosed herein state of the art methods may be used.

### Structural Characterization

Structural characterization of polyclonal anti-RSV antibody requires high resolution due to the complexity of the mixture (clonal diversity and glycosylation). Traditional approaches such as gel filtration, ion-exchange chromatography or electrophoresis may not have sufficient resolution to differentiate among the individual antibodies. Two-dimensional polyacrylamide gel electrophoresis (2D-PAGE) has been used for profiling of complex protein mixtures followed by mass spectrometry (MS) or liquid chromatography (LC)-MS (e.g., proteomics). 2D-PAGE, which combines separation on the basis of a protein's charge and mass, has proven useful for differentiating among polyclonal, oligoclonal and monoclonal immunoglobulin in serum samples. However, this method has some limitations. Chromatographic techniques, in particular capillary and LC coupled to electrospray ionization MS are increasingly being applied for the analysis of complex peptide mixtures. LC-MS has been used for the characterization of monoclonal antibodies. The analysis of very complex samples requires more resolving power of the chromatographic system, which can be obtained by separation in two dimensions (or more). Such an approach could be based on ion-exchange in the first dimension and reversed-phase chromatography (or hydrophobic interaction) in the second dimension optionally coupled to MS.

### Functional Characterization

A mono- and polyclonal anti-RSV antibody can for example be characterized functionally through comparability studies with anti-RSV antibody with specificity towards the same target or a similar activity. Such studies can be performed in vitro as well as in vivo.

An in vitro functional characterization of a polyclonal antibody could for example be immunoprecipitation which is a highly specific technique for the analytical separation of target antigens from crude cell lysates. By combining immunoprecipitation with other techniques, such as SDS-PAGE followed by protein staining (Coomassie Blue, silver staining or biotin labeling) and/or immunoblotting, it is possible to detect and quantify antigens e.g., and thus evaluate some of the functional properties of the antibodies. Although this method does not give an estimate of the number of antibody molecules nor their binding affinities, it provides a visualization of the target proteins and thus the specificity. This method can likewise be used to monitor potential differences of the antibodies toward antigens (the integrity of the clonal diversity) during the expression process.

An in vivo functional characterization of a mono- or polyclonal antibody could for example be infection studies. An experimental animal such as a mouse can for example be infected with RSV, towards which a polyclonal anti-RSV antibody has been developed. The degree to which the infection can be inhibited will indicate functionality of the polyclonal anti-RSV antibody.

### Therapeutic Compositions

In an embodiment of the invention, a pharmaceutical composition comprising a recombinant mono- and polyclonal anti-RSV antibody as it active ingredient is intended for the treatment or prevention of a disease in a mammal, preferably together with a pharmaceutically acceptable excipient.

The pharmaceutical compositions of the present invention are prepared in a manner known per se, for example, by means of conventional dissolving, lyophilising, mixing, granulating or confectioning processes. The pharmaceutical compositions may be formulated according to conventional pharmaceutical practice (see for example, in Remington: The Science and Practice of Pharmacy (20th ed.), ed. A. R. Gennaro, 2000, Lippincott Williams & Wilkins, Philadelphia, Pa. and Encyclopedia of Pharmaceutical Technology, eds. J. Swarbrick and J. C. Boylan, 1988-1999, Marcel Dekker, New York, N.Y.).

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example pre-servatives, stabilisers, wetting and/or emulsifying agents, solubilisers, salts for regulating the osmotic pressure and/or buffers, and are prepared in a manner known per se, for example by means of conventional dissolving or lyophilising processes. The said solutions or suspensions may comprise viscosity-increasing substances, such as sodium carboxymethylcellulose, carboxymethylcellulose, dextran, poly vinylpyrrolidone or gelatin.

The injection compositions are prepared in customary manner under sterile conditions; the same applies also to introducing the compositions into ampoules or vials and sealing the containers.

The pharmaceutical compositions may comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, drages, tablets or capsules.

Therapeutic uses of the Compositions According to the Invention

The pharmaceutical compositions made by the methods of the present invention according to the present invention may be used for the treatment, amelioration or prevention of a RSV infection in a mammal.

One aspect of the present invention is a method for disease treatment, amelioration or prophylaxis in an animal, wherein an effective amount of the recombinant polyclonal anti-RSV antibody or antibody fragment is administered.

### EXAMPLES

The following examples illustrate the invention, but should not be viewed as limiting the scope of the invention.

### Example 1

### Cloning and Sequencing of Human Anti-RSV Antibodies

In the present Example the isolation, screening, selection and banking of clones containing cognate V_{H} and V_{L} pairs expressed as full-length antibodies with anti-RSV specificity is illustrated. Cloning and linking of cognate pairs was carried out using Symplex™ cognate pairs cloning technology (Mejier et al, 2006, J. Mol. Biol, 358:764-772; WO 2005/042774). The cloning, characterization, and functional testing of human anti-RSV antibodies is described in co-pending PCT/DK2007/000113.

### Donors

Briefly, a total of 89 donors were recruited among the employees and parents of the children who were hospitalized at the Department of Paediatrics at Hvidovre Hospital (Denmark) during the RSV season. An initial blood sample of 18 ml was drawn, CD19⁺ B cells were purified and screened for the presence of anti-RSV antibodies using ELISpot and the frequency of plasma cells was determined by FACS analysis.

Eleven donors were found positive in the screening of the initial blood samples and a second blood sample of 450 ml was collected from ten of these. The plasma blasts were single-cell sorted and ELISpot was performed on a fraction of the CD 19 positive B cells.

Four donors with ELISpot frequencies in the second blood donation between 0.2 and 0.6% RSV specific plasma cells (IgG⁺ and IgA⁺) of the total plasma cell population were identified. These frequencies were considered high enough to proceed to linkage of cognate V_{H} and V_{L} pairs.

### Isolation of Cognate V_{H} and V_{L} Coding Pairs

The nucleic acids encoding the antibody repertoires were isolated from the single cell-sorted plasma cells from the five donors, by multiplex overlap-extension RT-PCR. The multiplex overlap-extension RT-PCR creates a physical link between the heavy chain variable region gene fragment (V_{H}) and the full-length light chain (LC). The protocol was designed to amplify antibody genes of all V_{H}-gene families and the kappa light chain, by using two primer sets, one for V_{H} amplification and one for the kappa LC amplification. Following the reverse transcription and multiplex overlap-extension PCR, the linked sequences were subjected to a second PCR amplification with a nested primer set.

Each donor was processed individually, and 1480 to 2450 overlap products were generated by the multiplex overlap-extension RT-PCR. The generated collection of cognate linked V_{H} and V_{L} coding pairs from each donor were pooled and inserted into a mammalian IgG expression vector. The generated repertoires were transformed into E. coli, and consolidated into twenty 384-well master plates and stored. The repertoires constituted between 1×10⁶ and 3.6×10⁶ clones per donor.

### Characterization of the Antigen Specificity of the Individual Antibodies

The antibodies identified during screening were validated by assessing their binding specificity to single RSV antigens (recombinant G protein, recombinant or purified F protein) or peptide fragments thereof (conserved region and cystein-core motif of protein G, subtype A and B, and the extracellular domain of SH protein, subtype A and B) by FLISA, ELISA and surface plasmon resonance (SPR; Biacore). The epitope specificities were determined in ELISA by competition with well-characterized commercial antibodies, some of which are shown in Table 2. Not necessarily all the antibodies shown in Table 2 were used in the characterization of each individual antibody of the present invention. Briefly, the antibodies or antibody fragments used for epitope blocking were incubated with the immobilized antigen (RSV Long particles, HyTest) in large excess, i.e. concentrations 100 times the ones giving 75% maximum binding, as determined empirically (Ditzel et al., J. Mol. Biol. 1997, 267:684-695). Following washing, the individual antibody clones were incubated with the blocked antigen at various concentrations and any bound human IgG was detected using a Goat-anti-Human HRP conjugate (Serotec) according to standard ELISA protocols. Epitope specificities were further characterized by pair-wise competition between different antibody clones in Biacore using saturating concentrations (empirically determined) of both blocking and probing antibodies. Purified F or G protein immobilized by direct amine coupling (Biacore) was used as antigen. In both the ELISA- and Biacore-based epitope mapping, the reduced binding following epitope blocking was compared to the uncompeted binding.

TABLE 2
Monoclonal antibodies for epitope mapping
of anti-F and anti-G antibodies

| | | Antigenic | | |
|---|---|---|---|---|
| MAb/Fab | Antigen | Site | Epitope (aa) | Ref. |
| 131-2a | F | F1 | F1a | 1, 2 |
| 9C5 | F | F1 | F1a | 5 |
| 92-11c | F | F1 | F1b | 1, 2 |
| 102-10b | F | F1 | F1c | 1, 2 |
| 133-1h | F | C | F2 | 1, 2, 3 |
| 130-8f | F | C | F2 (241/421) | 1, 2, 3, 4 |
| 143-6c | F | A/II | F3 | 1, 2, 3 |
| Palivizumab | F | A/II | (272) | 8 |
| 1153 | F | A/II | (262) | 3, 4 |
| 1142 | F | A/II | | 3 |
| 1200 | F | A/II | (272) | 2, 4 |
| 1214 | F | A/II | (276) | 3, 4 |
| 1237 | F | A/II | (276) | 3, 4 |
| 1129 | F | A/II | (275) | 3, 4 |
| 1121 | F | A/II | | 3 |
| 1112 | F | B/I | (389) | 3, 6 |
| 1269 | F | B/I | (389) | 3, 6 |
| 1243 | F | C | (241/421) | 3, 6 |
| Fab 19 | F | A/II | (266) | 7 |
| RSVF2-5 | F | IV | (429) | 4 |
| Mab19 | F | IV | (429) | 12 |
| 7.936 | F | V | (432-447) | 13 |
| 9.432 | F | VI | (436) | 13 |
| 63-10f | G (A) | G11 | GCRR (A171-187) | 1, 2 |
| 130-6d | G (A) | G12 | (A174-214) | 1, 2, 9 |
| 131-2g | G (A + B) | G13 | (150-173) | 1, 2, 9 |
| 143-5a | G (A + B) | G5a | | 2 |
| L9 | G (A + B) | A1/B1 | Conserved | 14, 15 |
| | | | (164-176) | |
| 8C5 | G | ND | | 5 |
| 1C2 | G (A) | ND | GCRR (A172-188) | 10, 11 |
| 3F4 | G (A) | ND | | 10, 11 |
| 4G4 | G (A) | ND | GCRR (A172-188) | 10, 11 |

The column “Antigen” indicates the RSV associated antigen bound by the Mab/Fab, and if a subtype specificity is known this is indicated in ( ). The column “Epitope (aa)” indicates the name of the epitope recognized by the MAb/Fab, further in ( ) amino acid positions resulting in RSV escape mutants, or peptides/protein fragments towards which binding has been show, are indicated.

The numbered references (Ref.) given in Table 2 correspond to:
1. Anderson et al., J. Clin. Microbiol. 1986, 23:475-480.
2. Anderson et al., J. Virol. 1988, 62:1232-4238.
3. Beeler & van Wyke Coelingh, J. Virol. 1989, 63:2941-2950.
4. Crowe et al., JID 1998, 177:1073-1076.
5. Sominina et al., Vestn Ross Akad Med Nauk 1995, 9:49-54.
6. Collins et al., Fields Virology, p. 1313-1351.
7. Crowe et al., Virology 1998, 252:373-375.
8. Zhao & Sullender, J. Virol. 2004, 79:3962-3968.
9. Sullender, Virology 1995, 209:70-79.
10. Morgan et al., J. Gen. Virol. 1987, 68:2781-2788.
11. McGill et al., J. Immunol. Methods 2005, 297:143-152.
12. Arbiza et al., J. Gen. Virol. 1992, 73:2225-2234.
13. Lopez et al. J. Virol. 1998, 72:6922-6928.
14. Walsh et al., J. Gen. Virol. 1989, 70:2953-2961.
15. Walsh et al., J. Gen. Virol. 1998, 79:479-487.

Furthermore, the antibody clones were also characterized in terms of binding to human laryngeal epithelial HEp-2 cells (ATCC CLL-23) infected with different RSV strains (Long and B1) by FACS. Briefly, HEp-2 cells were infected with either the RSV Long (ATCC number VR-26) strain or the RSV B1 (ATCC number VR-1400) strain in serum-free medium at a ratio of 0.1 pfu/cell for 24 (Long strain) or 48 h (B 1 strain). Following detachment and wash the cells were dispensed in 96-well plates and incubated with dilutions (4 pM-200 μM) of the individual anti-RSV antibodies for 1 h at 37° C. The cells were fixed in 1% formaldehyde and cell surface-bound antibody was detected by incubation with goat F(ab)₂ anti-human IgG-PE conjugate (Beckman Coulter) for 30 min at 4° C. Binding to mock-infected HEp-2 cells was similarly analyzed. Selected clones identified as protein G-specific were also tested for cross-reactivity with recombinant human fractalkine (CX3CL1; R&D systems) by ELISA. Anti-human CX3CL1/Fractalkine monoclonal antibody (R&D systems) was used as a positive control.

### Screening

IgG antibody-containing supernatants were obtained from CHO cells transiently transfected with DNA prepared from bacterial clones from the master plates and screened for binding to RSV antigen. Approximately 600 primary hits were sequenced and aligned. The majority fell in clusters of two or more members, but there were also clones that only were isolated once, so-called singletons. Representative clones from each cluster and the singletons were subjected to validation studies. A number of the primary hits were excluded from further characterization due to unwanted sequence features such as unpaired cysteins, non-conservative mutations, which are potential PCR errors, insertions and/or deletion of multiple codons, and truncations.

A total of 85 unique clones passed the validation. These are summarized in Table 3. Each clone number specifies a particular V_{H} and V_{L} pair. The IGHV and IGKV gene family is indicated for each clone and specifies the frame work regions (FR) of the selected clones. The amino acid sequence of the complementarity determining regions (CDR) of an antibody expressed from each clone are shown, where CDRH1, CDRH2, CDRH3 indicate the CDR regions 1, 2 and 3 of the heavy chain and CDRL1, CDRL2 and CDRL3 indicate the CDR regions 1, 2 and 3 of the light chain.

The complete variable heavy and light chain sequence can be established from the information in Table 3.

Further details to the individual columns of Table 3 are given below.

The IGHV and TGKV gene family names, were assigned according to the official HUGO/IMGT nomenclature (IMGT; Lefranc & Lefranc, 2001, The Immunoglobulin FactsBook, Academic Press). Numbering and alignments are according to Chothia (Al-Lazikani et al. 1997 J. Mol. Biol. 273:927-48). Clone 809 has a 2 codon insertion 5′ to CDRH1, which likely translates into an extended CDR loop. Clone 831 has a 1 codon deletion at position 31 in CDRH1.

The column “Ag” indicates the RSV associated antigen recognized by the antibody produced from the named clone, as determined by ELISA, FLISA and/or Biacore. “+” indicates that the clone binds to RSV particles and/or RSV-infected cells, but that the antigen has not been identified.

The column “Epitope” indicates the antigenic site or epitope recognized by the antibody produced from the named clone. “U” indicates that the epitope is unknown. UCI and UCII refer to unknown cluster I and II. Antibodies belonging to these clusters have similar reactivity profiles but have currently not been assigned to a particular epitope. Some antibodies recognize complex epitopes, such as A&C. Epitopes indicated in 0 have only been identified in ELISA.

TABLE 3
Summary of sequence and specificity of each unique validated clone.

| | | CDRH1 | CDRH2 | CDRH3 | | CDRL1 | CDRL2 | CDRL3 | | |
|---|---|---|---|---|---|---|---|---|---|---|
| | | 3 3 | 5 6 | 9 0 0 | | 2 3 3 | 5 | 8 9 9 | | |
| Clone | IGHV gene | 1ab2345 | 012abc3456789012345 | 234567890abcdefghijklmn123 | IGKV gene | 45678901abcdef234 | 0123456 | 89012345ab678 | Ag | Epitope |
| 735 | 4-59 | D--YDWS | NIN---YRGNTNYNPSLKS | CARDVGYGGGQYFAM--------DVW | 3-11 | RASQSVNS------HLA | NTFNRVT | CQQRSNWPPALTF | F | UCI |
| 736 | 3-30 | T--YGMH | FIRY--DGSTQDYVDSVKG | CAKDMDYYGSRSYSVTYYYGM--DVW | 1-39 | RASQRISN------HLN | GASTLQS | CQQSYRTPP-INF | F | A/II |
| 743 | 1-69 | T--YALT | RITP--MFDITNYAQKFQG | CARRGAVALVPAAEDPYYYGM--DVW | 2-28 | RSSQSLLHS-NGNNYLD | LASNRAS | CMQSLQT---PTF | G | Centr. dom |
| 744 | 1-2 | G--YYMH | WINT--SSGGTNYAQKFQG | CAREDGTMGTNSWYGWF------DPW | 3-20 | RASQSVSSS-----YLA | GASSRAT | CQQYDSSLSTWTF | F | A/II |
| 793 | 3-11 | D--YYMS | YINR--GGTTIYYADSVKG | CARGLILALPTATVELGAF----DIW | 1-39 | RASQSITG------YLN | ATSTLQS | CQQSYNT---LTF | G | Conserved |
| 794 | 1-18 | N--YGLN | WINA--YNDNTYYSPSLQG | CARSYRSQTDILTGRYKGPGDVFDNW | 1-12 | RASEGISS------WLA | AASTLQS | CQQTNSFP--YTF | G | GCRRA |
| 795 | 4-30-4 | SGDYYWS | YIF---HSGTTYYNPSLKS | CARDVDDFPVWGMNRYL------ALW | 3-20 | RASQSVSSS-----YLA | GASTGAT | CQQYGRTP--YTF | F | UCI |
| 796 | 3-30 | H--FGMH | IISY--DGNNVHYADSVKG | CAKDDVATDLAAYYYF-------DVW | 2-29 | RSSQSLLRS-DGKTFLY | EVSSRFS | CMQGLKIR--RTF | G | Conserved |
| 797 | 1-18 | R--FGIS | WISA--DNGNTYYAQNFQD | CVRGGVVTNRVYYYYGM------DVW | 1-9 | RASQGISS------YLA | AASTLQS | CQQVDTYP--LTF | G | GCRRA |
| 798 | 7-4-1 | S--YVMN | WINT--NTGDPAYAQDFTG | CAWFGEFGLF-------------DYW | 1-16 | RASQDINN------YLA | AASSLQS | CQQYKSLP--FTF | G | GCRRA |
| 799 | 3-30 | N--YGMH | VISY--DGRNKYFADSVKG | CARGSVQVWLHLGLF--------DNW | 1-5 | RASQSVSS------WVA | EASNLES | CQQYHSYSG-YTF | F | U |
| 800 | 3-33 | D--YGMN | VIWH--DGSNKNYLDSVKG | CARTPYEFWSGYYF---------DFW | 1D-13 | RASQGITD------SLA | AASRLES | CQQYSKSP--ATF | F | F1 |
| 801 | 3-33 | S--YAMH | VIYY--EGSNEYYADSVKG | CARKWLGM---------------DFW | 2-28 | RSSQSLLNS-NGFNYVD | LGSNRAS | CMQALETP--LTF | F | F1 |
| 802 | 3-48 | S--YEMN | YIGT--GGSDIYYGDSVKG | CARARPGYKV-------------DFW | 1-9 | RASQGISS------YLA | VASILES | CQQSKSFP--PTF | F | U |
| 803 | 4-30-4 | SGDYFWS | YIY---SSGSTFYNASLKS | CARGGTLYTTGGEM---------HIW | 3-20 | RASQTVSSS-----YLV | GASTRAT | CQQYGGSG--LTF | F | U |
| 804 | 3-64 | N--YAMH | ATST--DGGSTYYADSLKG | CARRFWGFGNFF-----------DYW | 3-20 | RASQSVSSG-----YLA | GASGRAT | CQQYFGSP--YTF | F | F1 |
| 805 | 4-59 | G--DFWS | YIY---YRGSTYYNPSLKS | CAREGHHSGSGDYYSFF------DYW | 1-39 | RASQGINT------YLN | AASSLQS | CQQSANSP--HTF | F | (F1) |
| 806 | 5-51 | S--YWIG | IVYP--GDSDTTYSPSFQG | CVRRGGFCTATGCYAGHWF----DPW | 3-20 | RASQSISSG-----YLA | GASHRAT | CQQYGSSL--WTF | + | U |
| 808 | 2-70 | TTRMSVS | RID---WDDDKYYSTSLKT | CARIVFHTSGGYYNPYM------DVW | 1-39 | RASQTIAS------YLS | TASSLQS | CQHSYNTP--YTF | F | (F1) |
| 809 | 5-51 | FVSTWIG | IINP--ADSDTRYSPSFQG | CARRAYDSGWHF-----------EHW | 3D-15 | RASQSVGS------KLA | GASTRAT | CQQYNNWPP-YTF | F | (F1) |
| 810 | 1-69 | N--YAIN | RIIP--VFDTTNYAQKFQG | CLRGSTRGWDTDGF---------DIW | 1D-17 | RASQGISN------YLV | AASSLQS | CLQHNISP--YTF | F | A/II |
| 811 | 1-46 | N--YYIH | VINP--NGGSTTSAQKFQD | CARQRSVTGGFDAWLLIPDAS--NTW | 4-1 | RSSETVLYTSKNQSYLA | WASTRES | CQQFFRSP--FTF | G | Conserved |
| 812 | 1-69 | S--YSIS | MILP--ISGTTNYAQTFQG | CARVFREFSTSTLDPYYF-----DYW | 3-20 | RASQSVSSS-----YIA | AASRRAT | CQHYGNSL--FTF | F | F1 |
| 813 | 5-51 | S--YWIG | IIYP--GDSDTRNSPSFQG | CVRQGGYYDRNGYHEKYAF----DIW | 1-5 | RASQSISS------WLA | KSSILES | CQHYNSYS--GTF | F | (F1) |
| 814 | 3-30-3 | D--YAMH | VISY--DGANEYYAESVKG | CARAGRSSMNEEVIMYF------DNW | 1-5 | RASQSIGS------RLA | DASSLES | CQQYNRDSP-WTF | G | Conserved |
| 816 | 3-23 | T--YAMT | VIRA--SGDSEIYADSVRG | CANIGQRRYCSGDHCYGHF----DYW | 2-28 | RSSQSLLHS-DGRYYVD | LASNRAS | CMQGLHTP--WTF | G | Conserved |
| 817 | 3-30 | T--HGMH | IISL--DGIKTHYADSVKG | CAKDHIGGTNAYFEWTVPF----DGW | 3-15 | WASQTIGG------NLA | GASTRAT | CQQYKNW---YTF | F | A/II |
| 818 | 2-70 | AGRVGVS | RID---WDDDKAFRTSLKT | CARTQVFASGGYYLYYL------DHW | 1-39 | RASQTIAS------YVN | AASNLQS | CQQSYSYRA-LTF | F | B/I/F1 |
| 819 | 4-30-4 | GADYYWS | FIY---DSGSTYYNPSLRS | CARDLGYGGNSYSHSYYYGL---DVW | 3-11 | RASQSVSS------SLA | DASYRVT | CQQRSNWPPGLTF | F | A/II |
| 822 | 5-51 | N--SWIG | IIYP--GDSTTTYTPSFQG | CARQGRGF---------------GLW | 1D-33 | QASQDITY------YLS | DVSNLER | CQQYDFLP--YTF | F | U |
| 823 | 4-b | SG-HFWG | SIF---HSGTTFHNPSLKS | CARVHGGGF--------------DHW | 1D-33 | QASQDIGD------SLN | DASNLET | CQHYVNLPPSFTF | + | U |
| 824 | 4-59 | N--YYWG | HIY---FGGNTNYNPSLQS | CARDSSNWPAGY-----------EDW | 1D-13 | RPSQDISS------ALA | GASTLDY | CQQFNTYP--FTF | F | F1&C |
| 825 | 1-18 | S--NGLS | WISA--SSGNKKYAPKFQG | CAKDGGTYVPYSDAF--------DFW | 4-1 | KSSQSVLYNSNNKNYLA | LASTREY | CQQYYQTP--LTF | F | UCI |
| 827 | 1-24 | A--LSKH | FFDP--EDGDTGYAQKFQG | CATVAAAGNF-------------DNW | 1-39 | RASQFISS------YLH | AASTLQS | CQQSYTNP--YTF | F | A&C/IV |
| 828 | 1-3 | T--NGLH | LINA--GNGDTRFSQKFQG | CARIAITMVRNPF----------DIW | 1-5 | RASQSIGS------WLA | KESNLES | CQQYKND---WTF | + | A&C |
| 829 | 2-70 | RNRMSVS | RID---WDDDKFYNTSLQT | CARTGIYDSSGYYLYYF------DYW | 1-39 | RASQSIAS------YLN | AASSLHS | CQHSYSTR--FTF | F | U (F1) |
| 830 | 1-18 | T--YGVS | WISA--YNGNTYYLQKLQG | CARDRVGGSSSEVLSRAKNYGL-DVW | 1-5 | RASQSVTS------ELA | KASSLES | CQQYNSFP--YTF | G | GCRRA |
| 831 | 1-3 | ---YAMH | WINV--GNGQTKYSQRFQG | CARRASQYGEVYGNYF-------DYW | 1-5 | RASQNIYN------WLA | DASTLES | CQQYNSLS--PTF | F | A/II |
| 833 | 3-30 | Y--IGMH | AISY--DGSNKQYADSVKG | CAKDDFGNSNGVFFMSRV-----AFW | 1-12 | RANQDIDN------YLA | GASKLQT | CQQAKSFP--FTF | G | Centr. dom |
| 834 | 1-18 | T--YGLN | WVSA--HNGNTYYAEKFHD | CVRGFNEQQLVPGLSFWF-----DYW | 1-12 | RASQGISK------RLA | GASSLQH | CQQADSFP--FTF | G | GCRRA |
| 835 | 1-18 | S--YGFS | WSSV--YNGDTNYAQKFHG | CARDRNVVLLPAAPFGGM-----DVW | 1-9 | RASQGISS------YLA | AASTLQS | CQQLNSYP--RTF | G | GCRR |
| 836 | 4-b | SG-HYWG | SIY---DSGNTYYTPSLKS | CARGSPGDAF-------------DIW | 1-12 | RASQGIGT------WLA | AASRLQS | CQQAYSFP--RTF | F | (A/II) |
| 838 | 3-30 | T--FGMH | VISY--DGNKKYYADSVKG | CAAQTPYFNESSGLV--------PDW | 1-27 | RASQGISN------YLA | AASTLQS | CQKYNSAP--QTF | G | Conserved |
| 839 | 3-30 | S--YGLH | EISY--DGGSKFYTDSVKG | CARDLGDGYTAWGWF--------DPW | 3-20 | RASQSVGGR-----SLA | DASNRAT | CQQYGSPP--WTF | G | GCRRA |
| 841 | 1-18 | S--FGIS | WISA--YNGNTDYAQRLQD | CTRDESMLRGVTEGFGPI-----DYW | 4-1 | RSSQSVLYSSNNKNYLA | WASTRAS | CQQFHSTP--RTF | G | GCRRA |
| 842 | 1-18 | R--YGIS | WISA--YNGNTYYAQNLQG | CVISFDSTIAAAEYF--------DYW | 1-5 | RASQTISN------SLA | KASTLES | CQQYNSFS--FTF | G | GCRRA |
| 843 | 1-18 | N--SGVS | WISA--YNGNTYYRQSLQD | CAREGHYSGSSSYQRDDAF----DIW | 1-16 | RASQGISN------YLA | TTSTLRS | CQQYHSFP--YTF | G | GCRRA |
| 845 | 1-18 | S--YGIS | WIGT--DNGNTYYAQKFQG | CARGGTIEATPEREYYYYGM---DVW | 1-9 | RASQGISS------YLA | AASTLQS | CQQLNTYP--LTF | G | GCRRA |
| 846 | 4-30-2 | SGGYSWS | YIY---HSGSTYYNPSLKS | CASRSFYGDY-------------VYW | 3-20 | RASQSVSSS-----YLA | GASSRAT | CQQYGSSP--FTF | F | U |
| 848 | 4-61 | SDKNYWS | RLY---PSGNTDYHPSLKS | CAKEGSGWYF-------------ESW | 1-5 | RASQGISA------WLA | DASTLAS | CQQYRSYS--YTF | F | U |
| 849 | 3-73 | G--STMH | RIRSKANSYATEYAASVKG | CTRHVGEMSTIWWYF--------DLW | 1-39 | RASQSISS------YLN | AASSLQS | CQQSYSTP--YTF | F | U |
| 850 | 1-3 | T--YTLH | LINA--ANGHTKYSQRFQG | CAKSGSHYGEVYGAYF-------DYW | 1-5 | RASQNIYN------WLA | DASSLES | CQQYNIYS--PTF | F | (A/II) |
| 851 | 1-18 | S--LGFS | WTSA--HNGNTYYAEEFQD | CARDRGPGYSDSSFYVF------DYW | 2-24 | RSSQSLVNS-DGNTYLS | QISKRFS | CMQATQFP--FTF | G | GCRRA |
| 852 | 1-69 | G--YTIH | RLVP--SLNIPNYAQKFQG | CTRAPRGSTASHLLF--------DYW | 1D-33 | QASQDVSY------YLN | DTSNLVT | CLQYHYLP--YTF | F | U |
| 853 | 5-51 | N--YWIG | VIFP--ADSDARYSPSFQG | CARPKYYFDSSGQFSEMYYF---DFW | 3-20 | RASQSVSSN-----YLA | GASSRAA | CQQYGNSP--LTF | G | Centr. dom |
| 855 | 1-18 | N--YAFS | WISG--SNGNTYYAEKFQG | CARDLLRSTYF------------DYW | 1D-12 | RASQAISN------WLA | AASSLQS | CQQADTFP--FTF | G | GCRRA |
| 856 | 1-18 | N--YGFS | WISA--YNGNTYYAQNLQG | CARDGNTAGVDMWSRDGF-----DIW | 2-40 | RSSQSLLDSNDGNTYLD | TFSYRAS | CMQRIEFP--YTF | G | GCRRA |
| 857 | 3-23 | S--YAMN | GISG--SGGSTYYGDSVKG | CAKEPWIDIVVASVISPYYYDGMDVW | 2-28 | RSSQSLLHR-NEYNYLD | WGSNRAS | CMQTLQTP--RTF | F | F1 |
| 858 | 1-69 | G--YTIS | RVVP--TLGFPNYAQKFQG | CARMNLGSHSGRPGF--------DMW | 1D-33 | QASQDISN------YLN | DATKLET | CQHFANLP--YTF | F | B/I/F1 |
| 859 | 3-33 | K--YGIH | VISY--DGSKKYFTDSVKG | CATGGGVNVTSWSDVEHSSSL--GYW | 1-27 | RASQGIRN------YLA | AASTLQS | CQRYNSAP--LTF | G | Conserved |
| 861 | 3-30 | S--YGMH | FIWN--DGSNKYYADSVKG | CVKDEVYDSSGYYLYYF------DSW | 1-39 | RASQIIAS------YLN | AASSLQS | CQQSYSTPI-FTF | F | F1 |
| 863 | 3-23 | S--YTMS | SISA--STVLTYYADSVKG | CAKDYDFWSGYPGGQYWFF----DLW | 3-11 | RTSQSVSS------YLA | DASNRAT | CQQRSDW---LTF | F | A/II |
| 866 | 1-18 | T--YGIS | WISA--DNGNTYYAQKFQG | CVRGGTYSSDVEYYYYGM-----DVW | 1-9 | RASQGISI------YLA | AASTLQT | CQQLNIYP--LTF | G | GCRRA |
| 867 | 1-69 | R--YTIH | RVVP--SLGIPNYAPKFQG | CARLTLGSYTGRPGF--------DSW | 1D-33 | QASQDINN------YLN | DATDLET | CQHFANLP--YTF | F | (F1) |
| 868 | 4-b | NA-YYWG | SIH---HSGSAYYNSSLKS | CARDTILTFGEPHWF--------DPW | 3-15 | RASQSIKN------NLA | GASARAT | CQEYNNWPL-LTF | G | Conserved |
| 869 | 3-30 | Y--YAMH | VISY--GETNKLYADSVKG | CARDLRYLTYYSGSGD-------DSW | 3-20 | RASQSLSDN-----YLA | GASSRPT | CQQYGTTP--ITF | G | Conserved |
| 870 | 4-59 | N--YYWS | EIS---NTWSTNYNPSLKS | CARGLFYDSGGYYLFYF------QHW | 1-39 | RASQRIAS------YLN | AASSLQS | CQQSYSTPI-YTF | F | (F1) |
| 871 | 3-33 | N--YGMH | VIWY--DDSNKQYGDSVKG | CARASEYSISWRHRGVL------DYW | 1D-33 | QASQGISN------YLN | DASNLES | CQQYDNFP--YTF | F | UCI |
| 874 | 3-30 | H--YGMH | VISH--DGNIKYSADSVKG | CHGEGYSTSWLGTAAL-------DYW | 1-27 | RASQGIRN------FLA | AASTLQS | CQKYNSAP--WTF | G | Conserved |
| 879 | 3-23 | A--YAMS | AISG--GGGTTYYADSVKG | CAKTRGYSYTWGDAF--------DLW | 3-15 | RASQSVTS------NLA | GASTRAT | CQQYNNWP--QTF | F | U |
| 880 | 2-5 | TSKLGVG | LVD---WDDDRRYRPSLKS | CAHSAYYTSSGYYLQYF------HHW | 1-39 | RASQTIAS------YVN | AASSLQS | CQQSYSFP--YTF | F | UCII |
| 881 | 3-48 | S--YEMT | HIGN--SGSMIYYADSVKG | CARSDYYDSSGYYLLYL------DSW | 1-39 | RASQTIAS------YVN | AASNLQS | CQQSYSVPR-LTF | F | UCII |
| 884 | 1-3 | N--FAMH | YINA--VNGNTQYSQKFQG | CARNNGGSAIIF-----------YYW | 1-39 | RSSQTISV------FLN | AASSLHS | CQESFSS---STF | F | U |
| 885 | 4-b | SN-YYWG | SMH---HSGSSYYKPSLKS | CARDLVVVTDISIKNYF------DPW | 3-11 | RASQSVTK------YLA | DASNRAT | CQHRRSW---PTF | + | U |
| 886 | 3-30 | S--YGMH | VISN--DGSNKYYADSVKG | CAKTTDQRLLVDWF---------DPW | 3-15 | RASQSVSS------NLA | SASTRAT | CQQYNMWPP-WTF | F | A/II |
| 887 | 2-70 | TSRMSVS | RID---WDDDKYYSTSLKT | CARTLVYAPDSYYLYYF------DYW | 1-39 | RASQTIAS------YVN | AASRLQS | CQQSYSIP--WTF | F | U (F1) |
| 888 | 4-39 | SSNFYWG | SIF---YSGTTYYNPSLKS | CARHGFRYCNNGVCSINLDAF--DIW | 2-28 | RSSQSLLRT-NGYNYLD | LGSIRAS | CMQSLQTS--ITF | G | GCRR |
| 889 | 1-18 | T--YGIS | WISA--YNGNTFYAQRLQG | CARDLRMLPGGLPTRRGM-----DVW | 1-5 | RASQSISS------WLA | KASSLES | CQQYNSYP--YTF | G | GCRRA |
| 890 | 1-46 | K--FYIH | IINP--SGGSTTYAQTFQD | CARGIREGGVSVEDWMLVYSWF-DPW | 1-39 | RASQNIRT------FIN | AASKLES | CQQGHSTP--YTF | G | Conserved |
| 891 | 3-30 | S--YTMH | VVSY--DGNHNDYADSVKG | CVRAPGSMGL-------------DVW | 2-28 | RSSQSLLHR-NGYNHLD | LGSNRAS | CMQALQTP--RTF | G | Centr. dom |
| 892 | 3-15 | N--AWMS | LIKSHFEGGATDYAAPVKG | CAPLGGPTPF-------------DYW | 1-17 | RAGQGIRN------DLG | GASTLQS | CLQHNSYP--WTF | + | U |
| 893 | 3-30 | I--YGMH | VISY--DGAKKFYANSVKG | CATASTYFYDSR-----------DYW | 2-24 | RSSRSLVHS-DGNTYLS | KISNRFS | CLQATQF---LTF | G | Conserved |
| 894 | 3-33 | D--YGMH | VIWH--DGSNIRYADSVRG | CARVPFQIWSGLYF---------DHW | 3-15 | RASQSVGN------NLA | GASTRAT | CQQYDKWP--ETF | F | UCI |
| 924 | 4-b | SE-YYWG | SVH---HSGSTYYNPSLKS | CARDRVALGVHYWYF--------DIW | 3-15 | RASQSVSS------HLA | GASTRAT | CQQYDNWL--PTF | G | Centr. dom |
| 955 | 1-46 | D--YCMH | ILNP--DGGTTFYAEKFQD | CAILIARAYCGLADGQEGDF---DTW | 1-5 | RASRSITS------WLA | KASSLQS | CQQYNSYP--LTF | SH | A2 aa42-64 |

The amino acid sequences from top to bottom in the column termed CDRH1 are set forth in the same order in SEQ ID NOs: 201-285. The amino acid sequences from top to bottom in the column termed CDRH2 are set forth in the same order in SEQ ID NOs. 286-370. The amino acid sequences from top to bottom in the column termed CDRH3 are set forth in the same order in SEQ ID NOs: 371-455. The amino acid sequences from top to bottom in the column termed CDRL1 are set forth in the same order in SEQ ID NOs. 456-540. The amino acid sequences from top to bottom in the column termed CDRL2 are set forth in the same order in SEQ ID NOs: 541-625. The amino acid sequences from top to bottom in the column termed CDRL3 are set forth in the same order in SEQ ID NOs. 626-710.

### Characterization of Binding Kinetics

The binding affinity for recombinant RSV antigens was determined by surface plasmon resonance for a number antibody clones. The analysis was performed with Fab fragments prepared by enzymatic cleavage of the full-length antibodies. Data for a number of high-affinity antibody clones with K_{D} values in the picomolar to nanomolar range is presented in Table 4. Fab fragments derived from commercially available Palivizumab (Synagis) were similarly analyzed for reference.

TABLE 4
Kinetic binding constants and affinities of selected clones.

| Fab clone | kₒₙ | k_{off} | t_{1/2} | K_{D} |
|---|---|---|---|---|
| (antigen) | (10⁵ M⁻¹ s⁻¹) | (10⁻⁵ l/s) | (min) | (pM) |
| 735 (F) | 4.07 | 9.18 | 130 | 226 |
| 810 (F) | 17.40 | 34.80 | 33 | 200 |
| 818 (F) | 1.92 | 2.20 | 530 | 115 |
| 817 (F) | 0.92 | 7.54 | 150 | 820 |
| 819 (F) | 3.56 | 4.99 | 230 | 140 |
| 825 (F) | 7.72 | 15.00 | 77 | 195 |
| 858 (F) | 4.97 | 0.34 | 3400 | 7 |
| 831 (F) | 3.72 | 42 | 28 | 1130 |
| 796 (G) | 8.33 | 40.3 | 28.67 | 480 |
| 811 (G) | 4.98 | 17.1 | 68 | 340 |
| 816 (G) | 20.20 | 17.80 | 65 | 90 |
| 838 (G) | 2.64 | 5.06 | 230 | 190 |
| 853 (G) | 17.7 | 140 | 8.25 | 790 |
| 859 (G) | 3.8 | 4.63 | 250 | 120 |
| Synagis (F) | 2.00 | 75.70 | 15 | 3780 |

### Sequences of Representative Human Anti-RSV Antibodies

The full sequences (DNA and deduced amino acid) of 44 selected clones which each express a unique antibody from a single cognate V_{H} and V_{L} gene sequence (clone nr 735, 736, 744, 793, 795, 796, 799, 800, 801, 804, 810, 811, 812, 814, 816, 817, 818, 819, 824, 825, 827, 829, 830, 831, 835, 838, 841, 853, 855, 856, 857, 858, 859, 861, 863, 868, 870, 871, 880, 881, 884, 886, 888, 894) are shown in SEQ ID NOs 1-176.

The 44 clones are charecterized by producing the following V_{H} sequences, which are set forth in SEQ ID NOs. 1-44:

Clone No. 735:
QVQLQESGPGLVKPSETLSLTCTVSNGAIGDYDWSWIRQSPGKGLEWIGNINYRG
NTNYNPSLKSRVTMSLRTSTMQFSLKLSSATAADTAVYYCARDVGYGGGQYFAMDVW
SPGTTVTVSS
Clone No. 736:
QVQLVESGGGVVQPGGSLRLSCTASGFTFSTYGMHWVRQAPGKGLEWVAFIRY
DGSTQDYVDSVKGRFTISRDNSKNMVYVQMNSLRVEDTAVYYCAKDMDYYGSRSYSV
TYYYGMDVWGQGTTVTVSS
Clone No. 744:
QVQLVQSGAEVKKPGASVKVSCKASGYTFSGYYMHWVRQAPGQGLEWMGWI
NTSSGGTNYAQKFQGRVTMTRDTSISTAHMELRRLRSDDTAVYYCAREDGTMGTNSW
YGWFDPWGQGTLVTVSS
Clone No. 793:
QVQLVESGGGLVKPGGSLRLSCAASGFPFGDYYMSWIRQAPGKGLEWVAYINR
GGTTIYYADSVKGRFTISRDNAKNSLFLQMNSLRAGDTALYYCARGLILALPTATVELG
AFDIWGQGTMVTVSS
Clone No. 795:
QVQLQESGPGLVKPSQTLSLTCTVSGASISSGDYYWSWIRQSPRKGLEWIGYIFHS
GTTYYNPSLKSRAVISLDTSKNQFSLRLTSVTAADTAVYYCARDVDDFPVWGMNRYLA
LWGRGTLVTVSS
Clone No. 796:
QVQLVESGGGVVQPGRSLRLSCAASGFSFSHFGMHWVRQVPGKGLEWVAIISYD
GNNVHYADSVKGRFTISRDNSKNTLFLQMNSLRDDDTGVYYCAKDDVATDLAAYYYF
DVWGRGTLVTVSS
Clone No. 799:
QVQLVESGGGVVQPGRSLKLSCEASGFNFNNYGMHWVRQAPGKGLEWVAVISY
DGRNKYFADSVKGRFIISRDDSRNTVFLQMNSLRVEDTAVYYCARGSVQVWLHLGLFD
NWGQGTLVTVSS
Clone No. 800:
QVQLVESGGAVVQPGRSLRLSCEVSGFSFSDYGMNWVRQGPGKGLEWVAVIWH
DGSNKNYLDSVKGRFTVSRDNSKNTLFLQMNSLRAEDTAVYYCARTPYEFWSGYYFDF
WGQGTLVTVSS
Clone No. 801:
QVQLVESGGGVVQPGRSLRLSCAASGFPFNSYAMHWVRQAPGKGLEWVAVIYY
EGSNEYYADSVKGRFTISRDNSKNTLYLQMDSLRAEDTAVYYCARKWLGMDFWGQGT
LVTVSS
Clone No. 804:
EVQLVESGGGLVRPGGSLRLSCSASGFTFSNYAMHWVRQAPGKRLEYVSATSTD
GGSTYYADSLKGTFTISRDNSKNTLYLQMSSLSTEDTAIYYCARRFWGFGNFFDYWGRG
TLVTVSS
Clone No. 810:
QVQLVQSGAEVKKSGSSVKVSCRASGGTFGNYAINWVRQAPGQGLEWVGRIIPV
FDTTNYAQKFQGRVTITADRSTNTAIMQLSSLRPQDTAMYYCLRGSTRGWDTDGFDIW
GQGTMVTVSS
Clone No. 811:
QVQLVQSGAVVETPGASVKVSCKASGYIFGNYYIHWVRQAPGQGLEWMAVINP
NGGSTTSAQKFQDRITVTRDTSTTTVYLEVDNLRSEDTATYYCARQRSVTGGFDAWLLI
PDASNTWGQGTMVTVSS
Clone No. 812:
QVQLVQSGAEMKKPGSSVKVSCKASGGSFSSYSISWVRQAPGRGLEWVGMILPIS
GTTNYAQTFQGRVIISADTSTSTAYMELTSLTSEDTAVYFCARVFREFSTSTLDPYYFDY
WGQGTLVTVSS
Clone No. 814:
QVQLVESGGGVVQPGKSVRLSCVGSGFRLMDYAMHWVRQAPGKGLDWVAVIS
YDGANEYYAESVKGRFTVSRDNSDNTLYLQMKSLRAEDTAVYFCARAGRSSMNEEVIM
YFDNWGLGTLVTVSS
Clone No. 816:
EVQLLESGGGLVQPGGSLRLSCVASGFTFSTYAMTWVRQAPGKGLEWVSVIRAS
GDSEIYADSVRGRFTISRDNSKNTVFLQMDSLRVEDTAVYFCANIGQRRYCSGDHCYGH
FDYWGQGTLVTVSS
Clone No. 817:
QVQLVESGGGVVQPGRSLRLSCAASGFGFNTHGMHWVRQAPGKGLEWLSIISLD
GIKTHYADSVKGRFTISRDNSKNTVFLQLSGLRPEDTAVYYCAKDHIGGTNAYFEWTVP
FDGWGQGTLVTVSS
Clone No. 818:
QVTLRESGPAVVKPTETLTLTCAFSGFSLNAGRVGVSWIRQPPGQAPEWLARIDW
DDDKAFRTSLKTRLSISKDSSKNQVVLTLSNMDPADTATYYCARTQVFASGGYYLYYL
DHWGQGTLVTVSS
Clone No. 819:
QVQLQESGPGLVKPSQTLSLTCTVSSGAISGADYYWSWIRQPPGKGLEWVGFIYD
SGSTYYNPSLRSRVTISIDTSKKQFSLKLTSVTAADTAVYYCARDLGYGGNSYSHSYYYG
LDVWGRGTTVTVSS
Clone No. 824:
QVQLQESGPGLVKPSETLSLTCTVSGGSIGNYYWGWIRQPPGKGLEWIGHIYFGG
NTNYNPSLQSRVTISVDTSRNQFSLKLNSVTAADTAVYYCARDSSNWPAGYEDWGQGT
LVTVSS
Clone No. 825:
QVQLVQSGAEVKKPGASVKVSCKVSGYTFTSNGLSWVRQAPGQGFEWLGWISA
SSGNKKYAPKFQGRVTLTTDISTSTAYMELRSLRSDDTAVYYCAKDGGTYVPYSDA [symbol] DF
WGQGTMVTVSS
Clone No. 827:
QVQLVQSGAEVKKPGASVKVSCRVSGHTFTALSKHWMRQGPGGGLEWMGFFD
PEDGDTGYAQKFQGRVTMTEDTATGTAYMELSSLTSDDTAVYYCATVAAAGNFDNWG
QGTLVTVSS
Clone No. 829:
QVTLKESGPALVKATQTLTLTCTFSGFSLSRNRMSVSWIRQPPGKALEWLARIDW
DDDKFYNTSLQTRLTISKDTSKNQVVLTMTNMDPVDTATYYCARTGIYDSSGYYLYYF
DYWGQGTLVTVSS
Clone No. 830:
QVQLVQSGAEVKVPGASVKVSCKASGYTFTTYGVSWVRQAPGQGLEWMGWIS
AYNGNTYYLQKLQGRVTMTTDTSTSTAYMELRGLRSDDTAMYYCARDRVGGSSSEVL
SRAKNYGLDVWGQGTTVTVSS
Clone No. 831:
QVQLVQSGAEVKKPGASVKVSCKASANIFTYAMHWVRQAPGQRLEWMGWINV
GNGQTKYSQRFQGRVTITRDTSATTAYMELSTLRSEDTAVYYCARRASQYGEVYGNYF
DYWGQGTLVTVSS
Clone No. 835:
QVQLVQSGAEVKRPGASVKVSCKASGYTFISYGFSWVRQAPGQGLEWMGWSSV
YNGDTNYAQKFHGRVNMTTDTSTNTAYMELRGLRSDDTAVYFCARDRNVVLLPAAPF
GGMDVWGQGTMVTVSS
Clone No. 838:
QVQLVESGGGVVQPGTSLRLSCAASGFTFSTFGMHWVRQAPGKGLEWVAVISY
DGNKKYYADSVKGRFTISRDNSKNTLYLQVNSLRVEDTAVYYCAAQTPYFNESSGLVP
DWGQGTLVTVSS
Clone No. 841:
QVQLVQSGAEVKKPGASVKVSCKASGYTFISFGISWVRQAPGQGLEWMGWISA
YNGNTDYAQRLQDRVTMTRDTATSTAYLELRSLKSDDTAVYYCTRDESMLRGVTEGFG
PIDYWGQGTLVTVSS
Clone No. 853:
EVQLVQSGAEVKKPGQSLKISCKTSGYIFTNYWIGWVRQRPGKGLEWMGVIFPA
DSDARYSPSFQGQVTISADKSIGTAYLQWSSLKASDTAIYYCARPKYYFDSSGQFSEMYY
FDFWGQGTLVTVSS
Clone No. 855:
QVQLVQSGPEVKKPGASVKVSCKASGYVLTNYAFSWVRQAPGQGLEWLGWISG
SNGNTYYAEKFQGRVTMTTDTSTSTAYMELRSLRSDDTAVYFCARDLLRSTYFDYWGQ
GTLVTVSS
Clone No. 856:
QVQLVQSGAEVKKPGASVKVSCKASGYTFSNYGFSWVRQAPGRGLEWMGWIS
AYNGNTYYAQNLQGRVTMTTDTSTTTAYMVLRSLRSDDTAMYYCARDGNTAGVDMW
SRDGFDIWGQGTMVTVSS
Clone No. 857:
EVQLLESGGGLVQPGGPLRLSCVASGFSFSSYAMNWIRLAPGKGLEWVSGISGSG
GSTYYGDSVKGRFTISRDNSKNTLYLQMNSLRAEDTAVYYCAKEPWIDIVVASVISPYY
YDGMDVWGQGTTVTVSS
Clone No. 858:
QVQLVQSGAEVKKPGSSVKVSCKASGGSFDGYTISWLRQAPGQGLEWMGRVVP
TLGFPNYAQKFQGRVTVTADRSTNTAYLELSRLTSEDTAVYYCARMNLGSHSGRPGFD
MWGQGTLVTVSS
Clone No. 859:
QVQLVESGGGVVQPGRSLRLSCAVSGSSFSKYGIHWVRQAPGKGLEWVAVISYD
GSKKYFTDSVKGRFTIARDNSQNTVFLQMNSLRAEDTAVYYCATGGGVNVTSWSDVEH
SSSLGYWGLGTLVTVSS
Clone No. 861:
QVQLVESGGGVVQPGGSLRLSCAASGFTFSSYGMHWVRQAPGKGLEWVAFIWN
DGSNKYYADSVKGRFTISRDNSKNTLYLQMNSLRAEDTAVYYCVKDEVYDSSGYYLYY
FDSWGQGTLVTVSS
Clone No. 863:
EVQLLESGGGLVQPGGSLRLSCAASGFTFSSYTMSWVRQAPGKGLEWVSSISAST
VLTYYADSVKGRFTISRDNSKNTLYLQMSSLRAEDTAVYYCAKDYDFWSGYPGGQYW
FFDLWGRGTLVTVSS
Clone No. 868:
QVQLQESGPGLVTPSETLSVTCTVSNYSIDNAYYWGWIRQPPGKGLEWIGSIHHS
GSAYYNSSLKSRATISIDTSKNQFSLNLRSVTAADTAVYYCARDTILTFGEPHWFDPWGQ
GTLVTVSS
Clone No. 870:
QVQLQESGPGLVKPSETLSLTCTVSGDSISNYYWSWIRQPPGKGLEWIGEISNTWS
TNYNPSLKSRVTISLDMPKNQLSLKLSSVTAADTAVYYCARGLFYDSGGYYLFYFQHW
GQGTLVTVSS
Clone No. 871:
QVQLVESGGGVVQPGRSLRVSCAASGFTFSNYGMHWVRQAPGKGLEWVAVIW
YDDSNKQYGDSVKGRFTISRDNSKSTLYLQMDRLRVEDTAVYYCARASEYSISWRHRG
VLDYWGQGTLVTVSS
Clone No. 880:
QITLKESGPTLVRPTQTLTLTCTFSGFSLSTSKLGVGWIRQPPGKALEWLALVDW
DDDRRYRPSLKSRLTVTKDTSKNQVVLTMTNMDPVDTATYYCAHSAYYTSSGYYLQYF
HHWGPGTLVTVSS
Clone No. 881:
EVQLVESGGGVVQPGGSLRLSCEVSGFTFNSYEMTWVRQAPGKGLEWVSHIGNS
GSMIYYADSVKGRFTISRDNAKNSLYLQMNSLRVEDTAVYYCARSDYYDSSGYYLLYL
DSWGHGTLVTVSS
Clone No. 884:
QVQLVQSGAEVRKPGASVKVSCKASGHTFINFAMHWVRQAPGQGLEWMGYIN
AVNGNTQYSQKFQGRVTFTRDTSANTAYMELSSLRSEDTAVYYCARNNGGSAIIFYYW
GQGTLVTVSS
Clone No. 886:
QVQLVESGGGVVQPGRSLRLSCAASGFSFSSYGMHWVRQAPGKGLEWVAVISN
DGSNKYYADSVKGRFTISRDNSKKTMYLQMNSLRAEDTAVYFCAKTTDQRLLVDWFD
PWGQGTLVTVSS
Clone No. 888:
QLQLQESGPGLVKPSETLSLTCTASGGSINSSNFYWGWIRQPPGKGLEWIGSIFYS
GTTYYNPSLKSRVTISVDTSKNQFSLKLSPVTAADTAVYHCARHGFRYCNNGVCSINLD
AFDIWGQGTMVTVSS
Clone No. 894:
QVQLVESGGGVVQPGKSLRLSCAASGFRFSDYGMHWVRQAPSKGLEWVAVIW
HDGSNIRYADSVRGRFSISRDNSKNTLYLQMNSMRADDTAFYYCARVPFQIWSGLYFDH
WGQGTLVTVSS

[symbol]

indicates data missing or illegible when filed

These V_{H} amino acid sequences are in the clones encoded by the following nucleic acid sequences, which are also set forth as SEQ ID NOs. 45-88:

Clone No. 735:
caggtgcagctgcaggagtcgggcccaggactggtgaagccttcggagaccctgtccctcacgtgcactgtgtctaatggcgc
catcggcgactacgactggagctggattcgtcagtccccagggaagggactggagtggattgggaacataaattacagagggaacaccaa
ctacaacccctccctcaagagtcgagtcaccatgtccctacgcacgtccacgatgcagttctccctgaagctgagctctgcgaccgctgcgg
acacggccgtctattactgtgcgagagatgtaggctacggtggcgggcagtatttcgcgatggacgtctggagcccagggaccacggtca
ccgtctcgagt
Clone No. 736:
caggtgcagctggtggagtctgggggaggcgtggtccagcctggggggtccctgagactctcctgtacagcgtctggattcac
cttcagtacctatggcatgcactgggtccgccaggctcccggcaaggggctggaatgggtggcatttatacggtatgatggaagtactcaag
actatgtagactccgtgaagggccgattcaccatctccagagacaattccaagaatatggtgtatgtgcagatgaacagcctgagagttgag
gacacggctgtctattactgtgcgaaagacatggattactatggttcgcggagttattctgtcacctactactacggaatggacgtctggggcc
aagggaccacggtcaccgtctcgagt
Clone No. 744:
caggtgcagctggtgcagtctggggctgaggtgaagaagcctggggcctcagtgaaggtctcctgcaaggcttctggatacac
cttcagcggctattatatgcactgggtgcgacaggcccctggacaagggcttgagtggatgggatggatcaacactagcagtggtggcaca
aactatgcgcagaagtttcagggcagggtcaccatgaccagggacacgtccatcagcacagcccacatggaactgaggaggctgagatct
gacgacacggccgtgtattattgtgcgagagaggacggcaccatgggtactaatagttggtatggctggttcgacccctggggccagggaa
ccctggtcaccgtctcgagt
Clone No. 793:
caggtgcagctggtggagtctgggggaggcttggtcaagcctggggggtccctgagactctcctgtgcggcctctggattcccc
ttcggtgactactacatgagctggatccgccaggctccagggaagggactggagtgggttgcatacattaatagaggtggcactaccatata
ctacgcagactctgtgaagggccgattcaccatctccagggacaacgccaagaactccctgtttctgcaaatgaacagcctgagagccggg
gacacggccctctattactgtgcgagagggctaattctagcactaccgactgctacggttgagttaggagcttttgatatctggggccaaggg
acaatggtcaccgtctcgagt
Clone No. 795:
caggtgcagctgcaggagtcgggcccaggactggtgaagccttcacagaccctgtccctcacctgcactgtctctggtgcctcc
atcagcagtggtgattattactggagttggatccgtcagtctccaaggaagggcctggagtggattgggtacatcttccacagtgggaccac
gtactacaacccgtccctcaagagtcgagctgtcatctcactggacacgtccaagaaccaattctccctgaggctgacgtctgtgactgccgc
agacacggccgtctattattgtgccagagatgtcgacgattttcccgtttggggtatgaatcgatatcttgccctctggggccggggaaccctg
gtcaccgtctcgagt
Clone No. 796:
caggtgcagctggtggagtctgggggaggcgtggtccagcctgggaggtccctgagactctcctgtgcagcctctggattcag
cttcagtcactttggcatgcactgggtccgccaggttccaggcaaggggctggagtgggtggcaattatatcatatgatgggaataatgtaca
ctatgccgactccgtaaagggccgattcaccatctccagagacaattccaagaacacgctgtttctgcaaatgaacagcctgagagatgacg
acacgggtgtgtattactgtgcgaaggacgacgtggcgacagatttggctgcctactactacttcgatgtctggggccgtggcaccctggtc
accgtctcgagt
Clone No. 799:
caggtgcagctggtggagtctgggggcggcgtggtccagcctgggaggtccctgaaactctcttgtgaagcctctggattcaac
ttcaataattatggcatgcactgggtccgccaggcaccaggcaaggggctggagtgggtggcagttatttcatatgacggaagaaataagta
ttttgctgactccgtgaagggccgattcatcatctccagagacgattccaggaacacagtgtttctgcaaatgaacagcctgcgagttgaagat
acggccgtctattactgtgcgagaggcagcgtacaagtctggctacatttgggactttttgacaactggggccagggaaccctggtcaccgt
ctcgagt
Clone No. 800:
caggtgcagctggtggagtctgggggagccgtggtccagcctgggaggtccctgagactctcctgtgaagtgtctggattcagtt
tcagtgactatggcatgaactgggtccgccagggtccaggcaaggggctggagtgggtggcagttatatggcatgacggaagtaataaaa
attatctagactccgtgaagggccgattcaccgtctccagagacaattccaagaacacattgtttctgcaaatgaacagcctgagagccgaag
acacggctgtatattactgtgcgaggacgccttacgagttttggagtggctattactttgacttctggggccagggaaccctggtcaccgtctc
gagt
Clone No. 801:
caggtgcagctggtggagtctgggggaggcgtggtccagcctgggaggtccctgagactctcctgtgcagcgtctggattccc
cttcaatagctatgccatgcactgggtccgccaggctccaggcaaggggctggagtgggtggcagtgatatattatgaagggagtaatgaat
attatgcagactccgtgaagggccgattcaccatctccagagacaattccaagaacactctgtatttgcaaatggatagcctgagagccgag
gacacggctgtctattactgtgcgaggaagtggctggggatggacttctggggccagggaaccctggtcaccgtctcgagt
Clone No. 804:
gaggtgcagctggtggagtctgggggaggcttggtccggcctggggggtccctgagactctcctgttcagcctctggattcacct
tcagtaactatgctatgcactgggtccgccaggctccagggaagagactggaatatgtttcagctactagtactgatggggggagcacatac
tacgcagactccctaaagggcacattcaccatctccagagacaattccaagaacacactgtatcttcaaatgagcagtctcagtactgaggac
acggctatttattactgcgcccgccgattctggggatttggaaacttttttgactactggggccggggaaccctggtcaccgtctcgagt
Clone No. 810:
caggtgcagctggtgcagtctggggctgaggtgaagaagtccgggtcctcggtgaaggtctcctgcagggcttctggaggcac
cttcggcaattatgctatcaactgggtgcgacaggcccctggacaagggcttgagtgggtgggaaggatcatccctgtctttgatacaacaa
actacgcacagaagttccagggcagagtcacgattaccgcggacagatccacaaacacagccatcatgcaactgagcagtctgcgacctc
aggacacggccatgtattattgtttgagaggttccacccgtggctgggatactgatggttttgatatctggggccaagggacaatggtcaccgt
ctcgagt
Clone No. 811:
caggttcagctggtgcagtctggggctgtcgtggagacgcctggggcctcagtgaaggtctcctgcaaggcatctggatacatct
tcggcaactactatatccactgggtgcggcaggcccctggacaagggcttgagtggatggcagttatcaatcccaatggtggtagcacaact
tccgcacagaagttccaagacagaatcaccgtgaccagggacacgtccacgaccactgtctatttggaggttgacaacctgagatctgagg
acacggccacatattattgtgcgagacagagatctgtaacagggggctttgacgcgtggcttttaatcccagatgcttctaatacctggggcc
aggggacaatggtcaccgtctcgagt
Clone No. 812:
caggtgcagctggtgcagtctggggctgagatgaagaagcctgggtcctcggtgaaggtctcctgcaaggcttctggaggctcc
ttcagcagctattctatcagctgggtgcgacaggcccctggacgagggcttgagtgggtgggaatgatcctgcctatctctggtacaacaaa
ctacgcacagacatttcagggcagagtcatcattagcgcggacacatccacgagcacagcctacatggagctgaccagcctcacatctgaa
gacacggccgtgtatttctgtgcgagagtctttagagaatttagcacctcgacccttgacccctactactttgactactggggccagggaaccc
tggtcaccgtctcgagt
Clone No. 814:
caggtgcagctggtggagtctgggggaggcgtggtccagcctgggaagtccgtgagactctcctgtgtaggctctggcttcagg
ctcatggactatgctatgcactgggtccgccaggctccaggcaagggactggattgggtggcagttatttcatatgatggagccaatgaatac
tacgcagagtccgtgaagggccgattcaccgtctccagagacaattcagacaacactctgtatctacaaatgaagagcctgagagctgagg
acacggctgtgtatttctgtgcgagagcgggccgttcctctatgaatgaagaagttattatgtactttgacaactggggcctgggaaccctggt
caccgtctcgagt
Clone No. 816:
gaggtgcagctgttggagtctgggggaggcttggtccagcctggggggtccctgagactctcctgtgtagcctccggattcacct
ttagtacctacgccatgacctgggtccgccaggctccagggaaggggctggagtgggtctcagtcattcgtgctagtggtgatagtgaaatc
tacgcagactccgtgaggggccggttcaccatctccagagacaattccaagaacacggtgtttctgcaaatggacagcctgagagtcgagg
acacggccgtatatttctgtgcgaatataggccagcgtcggtattgtagtggtgatcactgctacggacactttgactactggggccagggaa
ccctggtcaccgtctcgagt
Clone No. 817:
caggtgcagctggtggagtctgggggaggcgtggtccaacctgggaggtccctgagactctcctgtgcagcctctggattcgg
cttcaacacccatggcatgcactgggtccgccaggctccaggcaaggggctggagtggctgtcaattatctcacttgatgggattaagaccc
actatgcagactccgtgaagggccgattcaccatctccagagacaattccaagaacacggtgtttctacaattgagtggcctgagacctgaa
gacacggctgtatattactgtgcgaaagatcatattggggggacgaacgcatattttgaatggacagtcccgtttgacggctggggccaggg
aaccctggtcaccgtctcgagt
Clone No. 818:
caggtcaccttgagggagtctggtccagcggtggtgaagcccacagaaacgctcactctgacctgcgccttctctgggttctcac
tcaacgccggtagagtgggtgtgagttggatccgtcagcccccagggcaggccccggaatggcttgcacgcattgattgggatgatgataa
agcgttccgcacatctctgaagaccagactcagcatctccaaggactcctccaaaaaccaggtggtccttacactgagcaacatggaccctg
cggacacagccacatattactgtgcccggacacaggtcttcgcaagtggaggctactacttgtactaccttgaccactggggccagggaac
cctggtcaccgtctcgagt
Clone No. 819:
caggtgcagctgcaggagtcgggcccaggactggtgaagccttcacagaccctgtccctcacctgcactgtctctagtggcgcc
atcagtggtgctgattactactggagttggatccgccagcccccagggaagggcctggagtgggttgggttcatctatgacagtgggagca
cctactacaacccgtccctcaggagtcgagtgaccatatcaatagacacgtccaagaagcagttctccctgaagctgacctctgtgactgcc
gcagacacggccgtgtattactgtgccagagatctaggctacggtggtaactcttactcccactcctactactacggtttggacgtctggggc
cgagggaccacggtcaccgtctcgagt
Clone No. 824:
caggtgcagctgcaggagtcgggcccaggactggtgaagccttcggagaccctgtccctcacctgcactgtctctggtggctcc
atcggaaattactactggggctggatccggcagcccccagggaagggacttgagtggattgggcatatctacttcggtggcaacaccaact
acaacccttccctccagagtcgagtcaccatttcagtcgacacgtccaggaaccagttctccctgaagttgaactctgtgaccgccgcggac
acggccgtgtattactgtgcgagggatagcagcaactggcccgcaggctatgaggactggggccagggaaccctggtcaccgtctcgagt
Clone No. 825:
caggttcagctggtgcagtctggagctgaggtgaagaagcctggggcctcagtgaaggtctcctgcaaggtttctggttacacct
ttaccagtaatggtctcagctgggtgcgacaggcccctggacaagggtttgagtggctgggatggatcagcgctagtagtggaaacaaaaa
gtatgccccgaaattccagggaagagtcaccttgaccacagacatttccacgagcacagcctacatggaactgaggagtctgagatctgac
gatacggccgtatattactgtgcgaaagatgggggcacctacgtgccctattctgatgcctttgatttctggggccaggggacaatggtcacc
gtctcgagt
Clone No. 827:
caggtccagctggtacagtctggggctgaggtgaagaagcctggggcctcagtgaaggtctcctgcagggtttccggacacac
tttcactgcattatccaaacactggatgcgacagggtcctggaggagggcttgagtggatgggattttttgatcctgaagatggtgacacagg
ctacgcacagaagttccagggcagagtcaccatgaccgaggacacagccacaggcacagcctacatggagctgagcagcctgacatctg
acgacacggccgtatattattgtgcaacagtagcggcagctggaaactttgacaactggggccagggaaccctggtcaccgtctcgagt
Clone No. 829:
caggtcaccttgaaggagtctggtcctgcgctggtgaaagccacacagaccctgacactgacctgcaccttctctgggttttcact
cagtaggaatagaatgagtgtgagctggatccgtcagcccccagggaaggccctggagtggcttgcacgcattgattgggatgatgataaa
ttctacaacacatctctgcagaccaggctcaccatctccaaggacacctccaaaaaccaggtggtccttacaatgaccaacatggaccctgt
ggacacagccacctattactgcgcacggactgggatatatgatagtagtggttattacctctactactttgactactggggccagggaaccctg
gtcaccgtctcgagt
Clone No. 830:
caggtgcagctggtgcagtctggagctgaggtgaaggtgcctggggcctcagtgaaggtctcctgcaaggcttctggttacacc
tttaccacttacggtgtcagctgggtgcggcaggcccctggacaagggcttgagtggatgggttggatcagcgcttacaatggtaacacata
ctatctacagaagctccagggcagagtcaccatgaccacagacacatccacgagcacagcctacatggagctgcggggcctgaggtctg
acgacacggccatgtattactgtgcgagagatcgtgttgggggcagctcgtccgaggttctatcgcgggccaaaaactacggtttggacgtc
tggggccaagggaccacggtcaccgtctcgagt
Clone No. 831:
caggttcagctggtgcagtctggggctgaggtgaagaagcctggggcctcagttaaggtttcctgcaaggcttctgcaaacatctt
cacttatgcaatgcattgggtgcgccaggcccccggacaaaggcttgagtggatgggatggatcaacgttggcaatggtcagacaaaatatt
cacagaggttccagggcagagtcaccattaccagggacacgtccgcgactacagcctacatggagctgagcaccctgagatctgaggac
acggctgtgtattactgtgcgaggcgtgcgagccaatatggggaggtctatggcaactactttgactactggggccagggaaccctggtcac
cgtctcgagt
Clone No. 835:
caggtgcagctggtgcagtctggagagaggtgaagaggcctggggcctcagtgaaggtctcctgcaaggcttcaggttacac
ctttatcagctatggtttcagctgggtgcgacaggcccctggacaagggcttgagtggatgggatggagcagcgtttacaatggtgacacaa
actatgcacagaagttccacggcagagtcaacatgacgactgacacatcgacgaacacggcctacatggaactcaggggcctgagatctg
acgacacggccgtgtatttctgtgcgagggatcgcaatgttgttctacttccagctgctccttttggaggtatggacgtctggggccaagggac
aatggtcaccgtctcgagt
Clone No. 838:
caggtgcagctggtggagtctgggggaggcgtggtccagccggggacttccctgagactctcctgtgcagcctctggattcacc
ttcagtacgtttggcatgcactgggtccgccaggctccaggcaaggggctggagtgggtggcagttatatcatatgatggaaataagaaata
ctatgcagactccgtgaagggccgattcaccataccagagacaattccaagaacacgctgtatctgcaagtgaacagcctgagagtcgag
gacacggctgtgtattactgtgcggcccaaactccatatttcaatgagagcagtgggttagtgccggactggggccagggcaccctggtca
ccgtctcgagt
Clone No. 841:
caggtgcagctggtgcagtctggagctgaggtgaagaagcctggggcctcagtgaaggtctcctgcaaggcttctggttacacc
tttatcagttttggcatcagctgggtgcgacaggcccctggacaaggacttgagtggatgggatggatcagcgcttacaatggtaacacaga
ctatgcacagaggctccaggacagagtcaccatgactagagacacagccacgagcacagcctacttggagctgaggagcctgaaatctg
acgacacggccgtgtactattgcactagagacgagtcgatgcttcggggagttactgaaggattcggacccattgactactggggccaggg
aaccctggtcaccgtctcgagt
Clone No. 853:
gaagtgcagctggtgcagtctggagcagaggtgaaaaagccggggcagtctctgaagatctcctgtaagacttctggatacatct
ttaccaactactggatcggctgggtgcgccagaggcccgggaaaggcctggagtggatgggggtcatctttcctgctgactctgatgccag
atacagcccgtcgttccaaggccaggtcaccatctcagccgacaagtccatcggtactgcctacctgcagtggagtagcctgaaggcctcg
gacaccgccatatattactgtgcgagaccgaaatattactttgatagtagtgggcaattctccgagatgtactactttgacttctggggccaggg
aaccctggtcaccgtctcgagt
Clone No. 855:
caggttcagctggtgcagtctggacctgaggtgaagaagcctggggcctcagtgaaggtctcctgcaaggcttctggttatgtgtt
gaccaactatgccttcagctgggtgcggcaggcccctggacaagggcttgagtggctgggatggatcagcggctccaatggtaacacata
ctatgcagagaagttccagggccgagtcaccatgaccacagacacatccacgagcacagcctacatggagctgaggagtctgagatctga
cgacacggccgtttatttctgtgcgagagatcttctgcggtccacttactttgactactggggccagggaaccctggtcaccgtctcgagt
Clone No. 856:
caggtgcagctggtgcagtctggagctgaggtgaagaagcctggggcctcagtgaaggtctcctgcaaggcttctggttacacc
ttttccaactacggtttcagctgggtgcgacaggcccctggacgagggcttgagtggatgggatggatcagcgcttacaatggtaacacata
ctatgcacagaacctccagggcagagtcaccatgaccacagacacatccacgaccacagcctacatggtactgaggagcctgagatctga
cgacacggccatgtattactgtgcgagagatggaaatacagcaggggttgatatgtggtcgcgtgatggttttgatatctggggccagggga
caatggtcaccgtctcgagt
Clone No. 857:
gaggtgcagctgttggagtctgggggaggcttggtacagcctggggggcccctgaggctctcctgtgtagcctctggattcagc
tttagcagctatgccatgaactggatccgcctggctccagggaaggggctggagtgggtctcaggtattagtggtagcggtggtagcactta
ctacggagactccgtgaagggccggttcaccatctccagagacaattccaagaacacgctgtatctgcaaatgaacagcctgagagccga
ggacacggccgtatattactgtgcgaaagagccgtggatcgatatagtagtggcatctgttatatccccctactactacgacggaatggacgt
ctggggccaagggaccacggtcaccgtctcgagt
Clone No. 858:
caggttcagctggtgcagtctggggctgaggtgaagaagcctgggtcctcggtgaaggtctcctgcaaggcctctggaggatcc
ttcgacggctacactatcagctggctgcgacaggcccctggacaggggcttgagtggatgggaagggtcgtccctacacttggttttccaaa
ctacgcacagaagttccaaggcagagtcaccgttaccgcggacagatccaccaacacagcctacttggaattgagcagactgacatctgaa
gacacggccgtatattactgtgcgaggatgaatctcggatcgcatagcgggcgccccgggttcgacatgtggggccaaggaaccctggtc
accgtctcgagt
Clone No. 859:
caggtgcagctggtggagtctgggggaggcgtggtccagcctgggaggtccttgagactctcctgtgcagtgtctggatccagc
ttcagtaaatatggcatacactgggtccgccaggctccaggcaaggggctggagtgggtggcagttatatcgtatgatggaagtaaaaagta
tttcacagactccgtgaagggccgattcaccatcgccagagacaattcccagaacacggtttttctgcaaatgaacagcctgagagccgagg
acacggctgtctattactgtgcgacaggagggggtgttaatgtcacctcgtggtccgacgtagagcactcgtcgtccttaggctactggggc
ctgggaaccctggtcaccgtctcgagt
Clone No. 861:
caggtgcagctggtggagtctgggggaggcgtggtccagcctggggggtccctgagactctcctgtgcagcgtctggattcac
cttcagtagctatggcatgcactgggtccgccaggctccaggcaaggggctggagtgggtggcatttatatggaatgatggaagtaataaat
actatgcagactccgtgaagggccgattcaccatctccagagacaattccaagaacacgctgtatctgcaaatgaacagcctgagagctga
ggacacggctgtgtattactgtgtgaaagatgaggtctatgatagtagtggttattacctgtactactttgactcttggggccagggaaccctgg
tcaccgtctcgagt
Clone No. 863:
gaggtgcagctgttggagtctgggggaggcttggtacagcctggggggtccctgagactctcctgtgcagcctctggattcacgt
ttagctcctataccatgagctgggtccgccaggctccagggaaggggctggagtgggtctcaagtattagtgctagtactgttctcacatacta
cgcagactccgtgaagggccgcttcaccatctccagagacaattccaagaacacgctgtatctgcaaatgagtagcctgagagccgagga
cacggccgtatattactgtgcgaaagattacgatttttggagtggctatcccgggggacagtactggttcttcgatctctggggccgtggcacc
ctggtcaccgtctcgagt
Clone No. 868:
caggtgcagctgcaggagtcgggcccaggactggtgacgccttcggagaccctgtccgtcacttgcactgtctctaattattccat
cgacaatgcttactactggggctggatccggcagcccccagggaagggtctggagtggataggcagtatccatcatagtgggagcgccta
ctacaattcgtccctcaagagtcgagccaccatatctatagacacgtccaagaaccaattctcgttgaacctgaggtctgtgaccgccgcaga
cacggccgtatattactgtgcgcgcgataccatcctcacgttcggggagccccactggttcgacccctggggccagggaaccctggtcacc
gtctcgagt
Clone No. 870:
caggtgcagctgcaggagtcgggcccaggactggtgaagccttcggagaccttgtccctcacctgcactgtctcaggtgactcc
atcagtaattactactggagttggatccggcagcccccagggaagggactggagtggattggagaaatatctaacacttggagcaccaatta
caacccctccctcaagagtcgagtcaccatatctctagacatgcccaagaaccagttgtccctgaagctgagctctgtgaccgctgcggaca
cggccgtatattactgtgcgagagggatttctatgacagtggtggttactacttgttttacttccaacactggggccagggcaccctggtcacc
gtctcgagt
Clone No. 871:
caggtgcagctggtggagtctgggggaggcgtggtccagcctgggaggtccctgagagtctcctgtgcagcgtctggattcac
cttcagtaactatggcatgcactgggtccgccaggctccaggcaaggggctggagtgggtggcagttatatggtatgatgacagtaataaac
agtatggagactccgtgaagggccgattcaccatctccagagacaattccaagagtacgctgtatctgcaaatggacagactgagagtcga
ggacacggctgtgtattattgtgcgagagcctccgagtatagtatcagctggcgacacaggggggtccttgactactggggccagggaacc
ctggtcaccgtctcgagt
Clone No. 880:
cagatcaccttgaaggagtctggtcctacgctggtgagacccacacagaccctcacactgacctgcaccttctctgggttctcact
cagcactagtaaactgggtgtgggctggatccgtcagcccccaggaaaggccctggagtggcttgcactcgttgattgggatgatgatagg
cgctacaggccatctttgaagagcaggctcaccgtcaccaaggacacctccaaaaaccaggtggtccttacaatgaccaacatggaccctg
tggacacagccacatattactgtgcacacagtgcctactatactagtagtggttattaccttcaatacttccatcactggggcccgggcaccctg
gtcaccgtctcgagt
Clone No. 881:
gaggtgcagctggtggagtctgggggaggcgtggtacagcctggaggctccctgagactctcctgtgaagtctccggattcacc
ttcaatagttatgaaatgacctgggtccgccaggccccagggaaggggctggagtgggtttcacacattggtaatagtggttctatgatatact
acgctgactctgtgaagggccgattcaccatctccagagacaacgccaagaactcactatatctgcaaatgaacagcctgagagtcgagga
cacggctgtttattactgtgcgaggtcagattactatgatagtagtggttattatctcctctacttagactcctggggccatggaaccctggtc
accgtctcgagt
Clone No. 884:
caggtgcagctggtgcagtctggggctgaggtgaggaagcctggggcctcagtgaaggtttcctgcaaggcttctggacatact
ttcattaactttgctatgcattgggtgcgccaggcccccggacaggggcttgagtggatgggatacatcaacgctgtcaatggtaacacaca
gtattcacagaagttccagggcagagtcacctttacgagggacacatccgcgaacacagcctacatggagctgagcagcctgagatctgaa
gacacggctgtgtattactgtgcgagaaacaatgggggctctgctatcattttttactactggggccagggaaccctggtcaccgtctcgagt
Clone No. 886:
caggtgcagctggtggagtctgggggaggcgtggtccagcctgggaggtccctgagactctcctgtgcagcctctggattcag
cttcagtagctatggcatgcactgggtccgccaggctccaggcaaggggctggagtgggtggcagttatatcaaatgatggaagtaataaat
actatgcagactccgtgaagggccgattcaccatctccagagacaattccaagaaaacgatgtatctgcaaatgaacagcctgagagctga
ggacacggctgtgtatttctgtgcgaagacaacagaccagcggctattagtggactggttcgacccctggggccagggaaccctggtcacc
gtctcgagt
Clone No. 888:
cagctgcagctgcaggagtcgggcccaggactggtgaagccatcggagaccctgtccctcacctgcactgcctctggtggctc
catcaacagtagtaatttctactggggctggatccgccagcccccagggaaggggctggagtggattgggagtatcttttatagtgggacca
cctactacaacccgtccctcaagagtcgagtcaccatatccgtagacacgtccaagaaccagttctccctgaagctgagccctgtgaccgcc
gcagacacggctgtctatcactgtgcgagacatggcttccggtattgtaataatggtgtatgctctataaatctcgatgcttttgatatctggg
gccaagggacaatggtcaccgtctcgagt
Clone No. 894:
caggtgcagctggtggagtctgggggaggcgtcgtccagcctggaaagtccctgagactctcctgtgcagcgtctggattcaga
ttcagtgactacggcatgcactgggtccggcaggctccaagcaaggggctggagtgggtggcagttatctggcatgacggaagtaatataa
ggtatgcagactccgtgaggggccgattttccatctccagagacaattccaagaacacgctgtatttgcaaatgaacagcatgagagccgac
gacacggctttttattattgtgcgagagtcccgttccagatttggagtggtctttattttgaccactggggccagggaaccctggtcaccgtctc
gagt

In the same clones, the complete amino acid sequences of the light chains (i.e. light chains including constant and variable regions) have the following amino acid sequences, which are also set forth as SEQ ID NOs: 89-132:

Clone No. 735:
EIVLTQSPATLSLSPGERATLSCRASQSVNSHLAWYQQKPGQAPRLLIYNTFNRVT
GIPARFSGSGSGTDFTLTISSLATEDFGVYYCQQRSNWPPALTFGGGTKVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Clone No. 736:
DIQMTQSPSSLSASVGDRVTFTCRASQRISNHLNWYQQKPGKAPKLLIFGASTLQS
GAPSRFSGSGSGTDFTLTITNVQPDDFATYYCQQSYRTPPINFGQGTRLDIKRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Clone No. 744:
EIVLTQSPGTLSLSPGERATLSCRASQSVSSSYLAWYQQKPGQAPRLLIYGASSRA
TGIPDRFSGSGSGTDFTLTISRLEPEDFAVYYCQQYDSSLSTWTFGQGTKVEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
Clone No. 793:
DIQMTQSPSSLSASVGDRVTITCRASQSITGYLNWYQQKPGKAPKLLIYATSTLQS
EVPSRFSGSGSGTDFTLTISSLQPEDFATYYCQQSYNTLTFGGGTKVEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
Clone No. 795:
EIVLTQSPGTLSLSPGERATLSCRASQSVSSSYLAWYQQKPGQAPRLLIHGASTGA
TGTPDRFSGSGSGTDFTLTISTLEPEDFAVYYCQQYGRTPYTFGQGTKLENKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Clone No. 796:
DIVMTQTPLSLSVTPGQPASISCRSSQSLLRSDGKTFLYWYLQKPGQSPQPLMYEV
SSRFSGVPDRFSGSGSGADFTLNISRVETEDVGIYYCMQGLKIRRTFGPGTKVEIKRTVAA
PSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
Clone No. 799:
DIQMTQSPSTLSASVGDRVTFSCRASQSVSSWVAWYQQKPGKAPKLLISEASNLE
SGVPSRFSGSGSGTEFTLTISSLQPEDFATYYCQQYHSYSGYTFGQGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Clone No. 800:
AIQLTQSPSSLSASVGDRVTLTCRASQGITDSLAWYQQKPGKAPKVLLYAASRLE
SGVPSRFSGRGSGTDFTLTISSLQPEDFATYYCQQYSKSPATFGPGTKVEIRRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Clone No. 801:
DIVMTQSPLSLPVTPGEPASISCRSSQSLLNSNGFNYVDWYLQKPGQSPQLLIYLG
SNRASGVPDRFSGSGSGTDFTLKISRVEAEDVGVYYCMQALETPLTFGGGTKVEIKRTV
AAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
Clone No. 804:
EIVLTQSPGTLSLSPGGRATLSCRASQSVSSGYLAWYQQKPGQAPRLLIYGASGR
ATGIPDRFSGSGSGTDFTLTISRLEPEDFAVYYCQQYFGSPYTFGQGTKLELKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Clone No. 810:
NIQMTQSPSAMSASVGDRVTITCRASQGISNYLVWFQQKPGKVPKRLIYAASSLQ
SGVPSRFSGSGSGTEFTLTISSLQPEDFATYYCLQHNISPYTFGQGTKLETKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Clone No. 811:
DIVMTQSPDSLAVSLGERATINCRSSETVLYTSKNQSYLAWYQQKARQPPKLLLY
WASTRESGVPARFSGSGSGTDFTLAISSLQAEDVAVYYCQQFFRSPFTFGPGTRLEIKRTV
AAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
Clone No. 812:
EIVLTQSPGTLSLSPGERVTLSCRASQSVSSSYIAWYQQKPGQAPRLVIYAASRRA
TGVPDRFSGSGSATDFTLTISRLEPEDLAVYYCQHYGNSLFTFGPGTKVDVKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Clone No. 814:
DIQMTQSPSTLSASVGDRVTITCRASQSIGSRLAWYQQQPGKAPKFLIYDASSLES
GVPSRFSGSGSGTEFTLTISSLQPEDLATYYCQQYNRDSPWTFGQGTKVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Clone No. 816:
DIVMTQSPLSLPVTPGEPASISCRSSQSLLHSDGRYYVDWYLQKPGQSPHLLIYLA
SNRASGVPDRFTGSGSGTDFTLKISRVEAEDVGVYYCMQGLHTPWTFGQGTKVDIKRTV
AAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
Clone No. 817:
EIVMTQSPATLSASPGERATLSCWASQTIGGNLAWYQQKPGQAPRLLIYGASTRA
TGVPARFSGSGSGTEFTLAISSLQSEDFAVYYCQQYKNWYTFGQGTKLELKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Clone No. 818:
DIQMTQSPSSLSASVGDRVTITCRASQTIASYVNWYQQKPGRAPSLLIYAASNLQS
GVPPRFSGSGSGTDFTLTISGLQPDDFATYYCQQSYSYRALTFGGGTKVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Clone No. 819:
EIVLTQSPATLSLSPGERATLSCRASQSVSSSLAWYQQTPGQAPRLLIYDASYRVT
GIPARFSGSGSGIDFTLTISSLEPEDFAVYYCQQRSNWPPGLTFGGGTKVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Clone No. 824:
AIQLTQSPSSLSASVGDTVTVTCRPSQDISSALAWYQQKPGKPPKLLIYGASTLDY
GVPLRFSGTASGTHFTLTISSLQPEDFATYYCQQFNTYPFTFGPGTKVDIKRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
Clone No. 825:
DIVMTQSPDSLAVSLGERATINCKSSQSVLYNSNNKNYLAWYQQKPGQPPKLLIH
LASTREYGVPDRFSGSGSGTDFALIISSLQAEDVAVYYCQQYYQTPLTFGQGTKVEIKRT
VAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
Clone No. 827:
DIQMTQSPSSLAASVGDRVTITCRASQFISSYLHWYQQRPGKAPKLLMYAASTLQ
SGVPSRFSGSGSGTDFTLTISSLQPEDFATYYCQQSYTNPYTFGQGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Clone No. 829:
DIQMTQSPSSLSASVGDRVTITCRASQSIASYLNWYQQKPGKAPKLLIYAASSLHS
GVPSRFSGSGSGTDFTLTISSLQPEDFATYYCQHSYSTRFTFGPGTKVDVKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Clone No. 830:
DIQMTQSPSTLSASVGDRVTITCRASQSVTSELAWYQQKPGKAPNFLIYKASSLES
GVPSRFSGSGSGTEFTLTISSLQPDDFATYYCQQYNSFPYTFGQGTKLEIKRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
Clone No. 831:
DIQMTQSPSTLSASVGDRLTITCRASQNIYNWLAWYQQKPGKAPKLLIYDASTLE
SGVPSRFSGSGSGTEFTLTISSLQPDDFATYYCQQYNSLSPTFGQGTKVEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Clone No. 835:
DIQLTQSPSFLSASLEDRVTITCRASQGISSYLAWYQQKPGKAPKLLLDAASTLQS
GVPSRFSGSGSGTEFTLTISSLQPEDFATYYCQQLNSYPRTFGQGTKVDIKRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
Clone No. 838:
DIQMTQSPSSLSASVGDRVSITCRASQGISNYLAWYQQKPGKVPKLLIYAASTLQS
GVPSRFSGSGSGTDFTLTISSLQPEDVATYYCQKYNSAPQTFGQGTKVEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Clone No. 841:
DIVMTQSPDSLAVSLGERATINCRSSQSVLYSSNNKNYLAWYQQKPGQPPKLLV
YWASTRASGVPDRFSGSGSGTDFTLTLSSLQAEDVAVYYCQQFHSTPRTFGQGTKVEIK
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
Clone No. 853:
EIVLTQSPGTLSLSPGERATLSCRASQSVSSNYLAWYQQKPGQAPRLLIYGASSRA
AGMPDRFSGSGSGTDFTLTISRLEPEDFAVYYCQQYGNSPLTFGGGTEVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Clone No. 855:
DIQMTQSPSSVSASVGDRVTITCRASQAISNWLAWYQQKPGKAPKLLIYAASSLQ
SGVPSRFSGSGSGTDFTLTISGLQPEDFATYYCQQADTFPFTFGPGTKVDIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Clone No. 856:
DIVMTQTPLSLPVTPGEPASISCRSSQSLLDSNDGNTYLDWYLQKPGQSPQLLIYT
FSYRASGVPDRFSGSGSGTDFTLKISRVEAEDVGVYYCMQRIEFPYTFGQGTKLEIKRTV
AAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
Clone No. 857:
DIVMTQSPLSLPVTPGEPASISCRSSQSLLHRNEYNYLDWYLQKPGQSPQLLIYWG
SNRASGVPDRFSGSGSGTDFTLKISRVEAEDVGVYYCMQTLQTPRTFGQGTKVEIKRTV
AAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
Clone No. 858:
DIQMTQSPSSVSASVGDRVTITCQASQDISNYLNWYQQKPGKAPKLLIFDATKLE
TGVPTRFIGSGSGTDFTVTITSLQPEDVATYYCQHFANLPYTFGQGTKLEIKRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Clone No. 859:
DIQMTQSPSSLSASVGDRVTITCRASQGIRNYLAWYQQKPGKVPKLLVFAASTLQ
SGVPSRFSGSGSGTDFTLTISSLQPEDVATYYCQRYNSAPLTFGGGTKVEIKRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
Clone No. 861:
DIQMTQSPSSLSASVGDRVTITCRASQIIASYLNWYQQKPGRAPKLLIYAASSLQS
GVPSRFSGSGSGTDFTLTISSLQPEDFATYYCQQSYSTPIFTFGPGTKVNIKRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
Clone No. 863:
EIVLTQSPATLSLSPGERATLSCRTSQSVSSYLAWYQQKPGQAPRLLIYDASNRAT
GIPARFSGSGSGTDFTLTISSLEPEDFAVYYCQQRSDWLTFGGGTKVEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
Clone No. 868:
EIVMTQSPATLSVSPGERATLSCRASQSIKNNLAWYQVKPGQAPRLLTSGASARA
TGIPGRFSGSGSGTDFTLTISSLQSEDIAVYYCQEYNNWPLLTFGGGTKVEIQRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Clone No. 870:
DIQMTQSPPSLSASVGDRVT TCRASQRIASYLNWYQQKPGRAPKLLIFAASSLQS
GVPSRFSGSGSGTDFTLTISSLQPEDYATYYCQQSYSTPIYTFGQGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Clone No. 871:
DIQMTQSPSSLSASVGDRVTITCQASQGISNYLNWYQQKPGKAPKLLIFDASNLES
EVPSRFSGRGSGTDFTFSISSLQPEDIATYFCQQYDNFPYTFGQGTKLEIKRTVAAPSVFIF
PPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
Clone No. 880:
DIQMTQSPSSLAASVGDRVTITCRASQTIASYVNWYQQKPGKAPNLLIYAASSLQ
SGVPSRFSGSGSGTDFTLTISSLQPEDFASYFCQQSYSFPYTFGQGTKLDIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
Clone No. 881:
DIQMTQSPSSLSASVGDRVTITCRASQTIASYVNWYQQKPGKAPKLLIYAASNLQ
SGVPSRFSGSGSGTDFTLTISSLQPEDFATYYCQQSYSVPRLTFGGGTKVDITRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Clone No. 884:
DIQMTQSPSSLSASVGDRVTITCRSSQTISVFLNWYQQKPGKAPKLLIYAASSLHS
AVPSRFSGSGSGTDFTLTISSLQPEDSATYYCQESFSSSTFGGGTKVEIKRTVAAPSVFIFPP
SDEQLKSGTASVVCLLNNFYPREAKVQWKVDNALQSGNSQESVTEQDSKDSTYSLSSTL
TLSKADYEKHKVYACEVTHQGLSSPVTKSFNRGEC
Clone No. 886:
EIVMTQSPATLSVSPGETATLSCRASQSVSSNLAWYQHKPGQAPRLLIHSASTRAT
GIPARFSGSGSGTEFTLTISSLQSEDFAVYYCQQYNMWPPWTFGQGTKVEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
Clone No. 888:
DIVMTQSPLSLPVTPGAPASISCRSSQSLLRTNGYNYLDWYLQKPGQSPQLLIYLG
SIRASGVPDRFSGSGSGTDFTLKISRVEAEDVGVYYCMQSLQTSITFGQGTRLEIKRTVAA
PSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
Clone No. 894:
EIVMTQSPATLSVSPGERATLSCRASQSVGNNLAWYQQRPGQAPRLLIYGASTRA
TGIPARFSGSGSGTEFTLTISSLQSEDFAVYYCQQYDKWPETFGQGTKVDIKRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC

The light chain encoding nucleic acid fragments in these clones have the following nucleic acid sequences, which are also provided as SEQ ID NOs: 133-176:

Clone No 735:
gaaattgtgttgacacagtctccagccaccctgtccttgtctccaggagaaagagccaccctctcctgcagggccagtcagagtg
ttaacagccacttagcctggtaccaacagaaacctggccaggctcccaggctcctcatctataatacattcaatagggtcactggcatcccag
ccaggttcagtggcagtgggtctgggacagacttcactctcaccatcagcagccttgcgactgaagattttggcgtttattactgtcagcagcg
tagcaactggcctcccgccctcactttcggcggagggaccaaagtggagatcaaacgaactgtggctgcaccatctgtcttcatcttcccgc
catctgatgagcagttgaaatctggaactgcctctgttgtgtgcctgctgaataacttctatcccagagaggccaaagtacagtggaaggtgg
ataacgccctccaatcgggtaactcccaggagagtgtcacagagcaggacagcaaggacagcacctacagcctcagcagcaccctgacg
ctgagcaaagcagactacgagaaacacaaagtctacgcctgcgaagtcacccatcagggcctgagctcgcccgtcacaaagagcttcaac
aggggagagtgt
Clone No 736:
gacatccagatgacccagtctccatcctccctgtctgcatctgtgggagacagagtcaccttcacttgccgggccagtcagagga
ttagcaaccatttaaattggtatcaacaaaagccagggaaagcccctaaactcctgatctttggtgcatccactcttcaaagtggggccccatc
aaggttcagtggcagtggatctgggacagatttcactctcaccatcactaatgtacaacctgacgattttgcaacttactactgtcaacagagtt
acagaactcccccgatcaacttcggccaagggacacgcctggacattaagcgaactgtggctgcaccatctgtcttcatcttcccgccatctg
atgagcagttgaaatctggaactgcctctgttgtgtgcctgctgaataacttctatcccagagaggccaaagtacagtggaaggtggataacg
ccctccaatcgggtaactcccaggagagtgtcacagagcaggacagcaaggacagcacctacagcctcagcagcaccctgacgctgag
caaagcagactacgagaaacacaaagtctacgcctgcgaagtcacccatcagggcctgagctcgcccgtcacaaagagcttcaacaggg
gagagtgt
Clone No 744:
gaaattgtgttgacgcagtctccaggcaccctgtctttgtctccaggggaaagagccaccctctcctgcagggccagtcagagtg
ttagcagcagctacttagcctggtatcagcagaaacctggccaggctcccaggctcctcatctatggtgcatccagcagggccactggcatc
ccagacaggttcagtggcagtgggtctgggacagacttcactctcaccatcagcagactggagcctgaagattttgcagtgtattactgtcag
cagtatgatagctcactttctacgtggacgttcggccaagggaccaaggtggaaatcaaacgaactgtggctgcaccatctgtcttcatcttcc
cgccatctgatgagcagttgaaatctggaactgcctctgttgtgtgcctgctgaataacttctatcccagagaggccaaagtacagtggaagg
tggataacgccctccaatcgggtaactcccaggagagtgtcacagagcaggacagcaaggacagcacctacagcctcagcagcaccctg
acgctgagcaaagcagactacgagaaacacaaagtctacgcctgcgaagtcacccatcagggcctgagctcgcccgtcacaaagagctt
caacaggggagagtgt
Clone No 793:
gacatccagatgacccagtctccatcctccctgtctgcatctgtaggagacagagtcaccatcacttgccgggcaagtcagagca
ttaccggctatttaaattggtatcagcagaaaccagggaaagcccctaaactcctgatctatgctacatccactttgcaaagtgaggtcccatc
aaggttcagtggcagtggatctgggacagatttcactctcaccatcagcagtcttcaacctgaagattttgcaacttactactgtcaacagagtt
ataataccctcactttcggcggagggaccaaggtggagatcaaacgaactgtggctgcaccatctgtcttcatcttcccgccatctgatgagc
agttgaaatctggaactgcctctgttgtgtgcctgctgaataacttctatcccagagaggccaaagtacagtggaaggtggataacgccctcc
aatcgggtaactcccaggagagtgtcacagagcaggacagcaaggacagcacctacagcctcagcagcaccctgacgctgagcaaagc
agactacgagaaacacaaagtctacgcctgcgaagtcacccatcagggcctgagctcgcccgtcacaaagagcttcaacaggggagagt
gt
Clone No 795:
gaaattgtgttgacgcagtctccaggcaccctgtctttgtctccaggggaaagagccaccctctcctgcagggccagtcagagtg
ttagcagcagctacttagcctggtatcagcagaaacctggccaggctcccaggctcctcatacatggcgcatccaccggggccactggcac
cccagacaggttcagtggcagtgggtctgggacagacttcactctcaccatcagtacactggagcctgaagattttgcagtgtattactgtca
gcaatatggtaggacaccgtacacttttggccaggggaccaagctggagaacaaacgaactgtggctgcaccatctgtcttcatcttcccgc
catctgatgagcagttgaaatctggaactgcctctgttgtgtgcctgctgaataacttctatcccagagaggccaaagtacagtggaaggtgg
ataacgccctccaatcgggtaactcccaggagagtgtcacagagcaggacagcaaggacagcacctacagcctcagcagcaccctgacg
ctgagcaaagcagactacgagaaacacaaagtctacgcctgcgaagtcacccatcagggcctgagctcgcccgtcacaaagagcttcaac
aggggagagtgt
Clone No 796:
gatattgtgatgacccagactccactctctctgtccgtcacccctggacagccggcctccatctcctgcaggtctagtcagagcct
cctgcgaagtgatggaaagacgtttttgtattggtatctgcagaagccaggccagtctccccaacccctaatgtatgaggtgtccagccggtt
ctctggagtgccagataggttcagtggcagcgggtcaggggcagatttcacactgaacatcagccgggtggagactgaggatgttgggat
ctattactgcatgcaaggtttgaaaattcgtcggacgtttggcccagggaccaaggtcgaaatcaagcgaactgtggctgcaccatctgtcttc
atcttcccgccatctgatgagcagttgaaatctggaactgcctctgttgtgtgcctgctgaataacttctatcccagagaggccaaagtacagtg
gaaggtggataacgccctccaatcgggtaactcccaggagagtgtcacagagcaggacagcaaggacagcacctacagcctcagcagc
accctgacgctgagcaaagcagactacgagaaacacaaagtctacgcctgcgaagtcacccatcagggcctgagctcgcccgtcacaaa
gagcttcaacaggggagagtgt
Clone No 799:
gacatccagatgacccagtctccttccaccctgtctgcatctgtaggagacagagtcaccttctcttgccgggccagtcagagtgtt
agtagttgggtggcctggtatcagcagaaaccaggaaaagcccctaagctcctgatctctgaggcctccaatttggaaagtggggtcccatc
ccggttcagcggcagtggatccgggacagaattcactctcaccatcagcagcctgcagcctgaagattttgcaacttattactgccaacagta
tcatagttactctgggtacacttttggccaggggaccaagttggaaatcaagcgaactgtggctgcaccatctgtcttcatcttcccgccatctg
atgagcagttgaaatctggaactgcctctgttgtgtgcctgctgaataacttctatcccagagaggccaaagtacagtggaaggtggataacg
ccctccaatcgggtaactcccaggagagtgtcacagagcaggacagcaaggacagcacctacagcctcagcagcaccctgacgctgag
caaagcagactacgagaaacacaaagtctacgcctgcgaagtcacccatcagggcctgagctcgcccgtcacaaagagcttcaacaggg
gagagtgt
Clone No 800:
gccatccagttgacccagtctccatcgtccctgtctgcatctgtaggcgacagagtcaccctcacttgccgggcgagtcagggca
ttaccgattctttagcctggtatcagcagaaaccagggaaagcccctaaggtcctgctctatgctgcttccagattggaaagtggggtcccatc
caggttcagtggccgtggatctgggacggatttcactctcaccatcagcagcctgcagcctgaagactttgcaacttattactgtcaacagtatt
ctaagtcccctgcgacgttcggcccagggaccaaggtggaaatcagacgaactgtggctgcaccatctgtcttcatcttcccgccatctgatg
agcagttgaaatctggaactgcctctgttgtgtgcctgctgaataacttctatcccagagaggccaaagtacagtggaaggtggataacgccc
tccaatcgggtaactcccaggagagtgtcacagagcaggacagcaaggacagcacctacagcctcagcagcaccctgacgctgagcaa
agcagactacgagaaacacaaagtctacgcctgcgaagtcacccatcagggcctgagctcgcccgtcacaaagagcttcaacaggggag
agtgt
Clone No 801:
gatattgtgatgacccagtctccactctccctgcccgtcacccctggagagccggcctccatctcctgcaggtctagtcagagcct
cctaaatagtaatggattcaactatgtggattggtacctgcagaagccagggcagtctccacaactcctgatctatttgggttctaatcgggcct
ccggggtccctgacaggttcagtggcagtggatcaggcacagattttacactgaaaatcagcagagtggaggctgaggatgttggggtttat
tactgcatgcaagctctagaaactccgctcactttcggcggagggaccaaggtggagatcaaacgaactgtggctgcaccatctgtcttcatc
ttcccgccatctgatgagcagttgaaatctggaactgcctctgttgtgtgcctgctgaataacttctatcccagagaggccaaagtacagtgga
aggtggataacgccctccaatcgggtaactcccaggagagtgtcacagagcaggacagcaaggacagcacctacagcctcagcagcac
cctgacgctgagcaaagcagactacgagaaacacaaagtctacgcctgcgaagtcacccatcagggcctgagctcgcccgtcacaaaga
gcttcaacaggggagagtgt
Clone No 804:
gaaattgtgttgacgcagtctccaggcaccctgtctttgtctccagggggaagagccaccctctcctgcagggccagtcagagtg
ttagcagcggctacttagcctggtaccagcagaaacctggccaggctcccaggctcctcatctatggtgcatccggcagggccactggcat
cccagacaggttcagtggcagtgggtctgggacagacttcactctcaccatcagcagactggagcctgaagattttgcagtgtattactgtca
gcagtattttggctcaccgtacacttttggccaggggaccaagctggagctcaaacgaactgtggctgcaccatctgtcttcatcttcccgcca
tctgatgagcagttgaaatctggaactgcctctgttgtgtgcctgctgaataacttctatcccagagaggccaaagtacagtggaaggtggata
acgccctccaatcgggtaactcccaggagagtgtcacagagcaggacagcaaggacagcacctacagcctcagcagcaccctgacgct
gagcaaagcagactacgagaaacacaaagtctacgcctgcgaagtcacccatcagggcctgagctcgcccgtcacaaagagcttcaaca
ggggagagtgt
Clone No 810:
aacatccagatgacccagtctccatctgccatgtctgcatctgtaggagacagagtcaccatcacttgtcgggcgagtcagggca
ttagtaattatttagtctggtttcagcagaaaccagggaaagtccctaagcgcctgatctatgctgcatccagtttgcaaagtggggtcccatca
aggttcagcggcagtggatctgggacagaattcactctcacaatcagcagcctgcagcctgaagattttgcaacttattactgtctacagcata
atatttccccttacacttttggccaggggaccaagctggagaccaaacgaactgtggctgcaccatctgtcttcatcttcccgccatctgatga
gcagttgaaatctggaactgcctctgttgtgtgcctgctgaataacttctatcccagagaggccaaagtacagtggaaggtggataacgccct
ccaatcgggtaactcccaggagagtgtcacagagcaggacagcaaggacagcacctacagcctcagcagcaccctgacgctgagcaaa
gcagactacgagaaacacaaagtctacgcctgcgaagtcacccatcagggcctgagctcgcccgtcacaaagagcttcaacaggggaga
gtgt
Clone No 811:
gacatcgtgatgacccagtctccagactccctggctgtgtctctgggcgagagggccaccatcaactgcaggtccagtgagact
gttttatacacctctaaaaatcagagctacttagcttggtaccagcagaaagcacgacagcctcctaaactactcctttactgggcatctacccg
ggaatccggggtccctgcccgattcagtggcagcggatctgggacagatttcactctcgccatcagcagcctgcaggctgaagatgtggca
gtttattactgtcagcaattttttaggagtcctttcactttcggccccgggaccagactggagattaaacgaactgtggctgcaccatctgtctt
catcttcccgccatctgatgagcagttgaaatctggaactgcctctgttgtgtgcctgctgaataacttctatcccagagaggccaaagtacag
tggaaggtggataacgccctccaatcgggtaactcccaggagagtgtcacagagcaggacagcaaggacagcacctacagcctcagcagc
accctgacgctgagcaaagcagactacgagaaacacaaagtctacgcctgcgaagtcacccatcagggcctgagctcgcccgtcacaaa
gagcttcaacaggggagagtgt
Clone No 812:
gaaattgtgttgacgcagtctccaggcaccctgtctttgtctccaggggaaagagttaccctctcttgcagggccagtcagagtgtt
agcagcagttacatagcctggtaccagcagaagcctggccaggctcccaggctcgtcatctatgctgcatcccgcagggccactggcgtc
ccagacaggttcagtggcagtgggtctgcgacagacttcactctcaccatcagtagactggagcctgaagatcttgcagtgtattactgtcag
cactatggtaactcactattcactttcggccctgggaccaaggtggatgtcaaacgaactgtggctgcaccatctgtcttcatcttcccgccatc
tgatgagcagttgaaatctggaactgcctctgttgtgtgcctgctgaataacttctatcccagagaggccaaagtacagtggaaggtggataa
cgccctccaatcgggtaactcccaggagagtgtcacagagcaggacagcaaggacagcacctacagcctcagcagcaccctgacgctg
agcaaagcagactacgagaaacacaaagtctacgcctgcgaagtcacccatcagggcctgagctcgcccgtcacaaagagcttcaacag
gggagagtgt
Clone No 814:
gacatccagatgacccagtctccctccaccctgtctgcatctgtcggagacagagtcaccatcacttgccgggccagtcagagta
ttggtagccggttggcctggtatcagcagcaaccagggaaagcccctaaattcctgatctatgatgcctccagtttggaaagtggggtcccat
caaggttcagcggcagtggatcagggacagaattcactctcaccatcagcagcctgcagccggaggatcttgcaacttattactgccaaca
gtacaatagagattctccgtggacgttcggccaagggaccaaggtggaaatcaagcgaactgtggctgcaccatctgtcttcatcttcccgc
catctgatgagcagttgaaatctggaactgcctctgttgtgtgcctgctgaataacttctatcccagagaggccaaagtacagtggaaggtgg
ataacgccctccaatcgggtaactcccaggagagtgtcacagagcaggacagcaaggacagcacctacagcctcagcagcaccctgacg
ctgagcaaagcagactacgagaaacacaaagtctacgcctgcgaagtcacccatcagggcctgagctcgcccgtcacaaagagcttcaac
aggggagagtgt
Clone No 816:
gatattgtgatgacccagtctccactctccctgcccgtcaccccaggagagccggcctccatctcctgcaggtctagtcagagcct
cctgcatagtgatggacgctactatgtggattggtacctgcagaagccagggcagtctccacacctcctgatctatttggcttctaatcgggcc
tccggggtccctgacaggttcactggcagtggatcaggcacagattttacactgaaaatcagcagagtggaggctgaggatgttggcgttta
ttactgcatgcaaggtctacacactccttggacgttcggccaggggaccaaggtggacatcaagcgaactgtggctgcaccatctgtcttcat
cttcccgccatctgatgagcagttgaaatctggaactgcctctgttgtgtgcctgctgaataacttctatcccagagaggccaaagtacagtgg
aaggtggataacgccctccaatcgggtaactcccaggagagtgtcacagagcaggacagcaaggacagcacctacagcctcagcagca
ccctgacgctgagcaaagcagactacgagaaacacaaagtctacgcctgcgaagtcacccatcagggcctgagctcgcccgtcacaaag
agcttcaacaggggagagtgt
Clone No 817:
gaaattgtaatgacacagtctccagccaccctgtctgcgtccccaggggaaagagccaccctctcctgttgggccagtcagacta
ttggaggcaacttagcctggtaccagcagaaacctggccaggctcccaggctcctcatctatggtgcatccaccagggccactggtgtccc
agccaggttcagtggcagtgggtctgggacagagttcactctcgccatcagcagcctgcagtctgaagattttgcagtttattactgtcagcag
tataaaaactggtacacttttggccaggggaccaagctggagctcaaacgaactgtggctgcaccatctgtcttcatcttcccgccatctgatg
agcagttgaaatctggaactgcctctgttgtgtgcctgctgaataacttctatcccagagaggccaaagtacagtggaaggtggataacgccc
tccaatcgggtaactcccaggagagtgtcacagagcaggacagcaaggacagcacctacagcctcagcagcaccctgacgctgagcaa
agcagactacgagaaacacaaagtctacgcctgcgaagtcacccatcagggcctgagctcgcccgtcacaaagagcttcaacaggggag
agtgt
Clone No 818:
gacatccagatgacccagtctccatcctccctgtctgcatctgtaggagacagagtcaccatcacttgccgggcaagtcagacca
ttgccagttacgtaaattggtaccaacaaaaaccagggagagcccctagtctcctgatctatgctgcatctaacttgcagagtggggtcccac
caaggttcagtggcagtggatctgggacagacttcactctcaccatcagcggtctgcaacctgacgattttgcaacttattactgtcaacagag
ttacagttatcgagcgctcactttcggcggagggaccaaggtggagatcaaacgaactgtggctgcaccatctgtcttcatcttcccgccatct
gatgagcagttgaaatctggaactgcctctgttgtgtgcctgctgaataacttctatcccagagaggccaaagtacagtggaaggtggataac
gccctccaatcgggtaactcccaggagagtgtcacagagcaggacagcaaggacagcacctacagcctcagcagcaccctgacgctga
gcaaagcagactacgagaaacacaaagtctacgcctgcgaagtcacccatcagggcctgagctcgcccgtcacaaagagcttcaacagg
ggagagtgt
Clone No 819:
gaaattgtgttgacacagtaccagccaccctgtcgttgtccccaggggaaagagccaccctctcctgcagggccagtcagagt
gttagcagctccttagcctggtaccaacagacacctggccaggctcccaggcttacatctatgatgcgtcctacagggtcactggcatccca
gccaggttcagtggcagtgggtctgggatagacttcactctcaccatcagcagcctagagcctgaagattttgcagtttactattgtcagcagc
gtagcaactggcctccggggctcactttcggcggggggaccaaggtggagatcaaacgaactgtggctgcaccatctgtcttcatcttcccg
ccatctgatgagcagttgaaatctggaactgcctctgttgtgtgcctgctgaataacttctatcccagagaggccaaagtacagtggaaggtg
gataacgccctccaatcgggtaactcccaggagagtgtcacagagcaggacagcaaggacagcacctacagcctcagcagcaccctgac
gctgagcaaagcagactacgagaaacacaaagtctacgcctgcgaagtcacccatcagggcctgagctcgcccgtcacaaagagcttca
acaggggagagtgt
Clone No 824:
gccatccagttgacccagtctccatcctccctgtctgcatctgttggagacacagtcaccgtcacttgccggccaagtcaggacat
tagcagtgctttagcctggtatcagcagaaaccagggaaacctcctaagctcctgatctatggtgcctccactttggattatggggtcccattaa
ggttcagcggcactgcatctgggacacatttcactctcaccatcagcagcctgcaacctgaagattttgcaacttattactgtcaacagtttaat
acttacccattcactttcggccctgggaccaaagtggatatcaaacgaactgtggctgcaccatctgtcttcatcttcccgccatctgatgagca
gttgaaatctggaactgcctctgttgtgtgcctgctgaataacttctatcccagagaggccaaagtacagtggaaggtggataacgccctcca
atcgggtaactcccaggagagtgtcacagagcaggacagcaaggacagcacctacagcctcagcagcaccctgacgctgagcaaagcag
actacgagaaacacaaagtctacgcctgcgaagtcacccatcagggcctgagctcgcccgtcacaaagagcttcaacaggggagagtgt
Clone No 825:
gacatcgtgatgacccagtctccagactccctggctgtgtctctgggcgagagggccaccatcaactgcaagtccagccagagt
gttttatacaactccaacaataagaactacttagcctggtatcagcagaaaccaggacagcctcctaagacctcattcacttggcatctaccc
gggaatacggggtccctgaccgattcagtggcagcgggtctgggacagatttcgctctcatcatcagcagcctgcaggctgaagatgtggc
agtttattactgtcaacaatattatcaaactcctctaacttttggccaggggaccaaggtggagatcaaacgaactgtggctgcaccatctgtct
tcatcttcccgccatctgatgagcagttgaaatctggaactgcctctgttgtgtgcctgctgaataacttctatcccagagaggccaaagtaca
gtggaaggtggataacgccctccaatcgggtaactcccaggagagtgtcacagagcaggacagcaaggacagcacctacagcctcagcag
caccctgacgctgagcaaagcagactacgagaaacacaaagtctacgcctgcgaagtcacccatcagggcctgagctcgcccgtcacaa
agagcttcaacaggggagagtgt
Clone No 827:
gacatccagatgacccagtctccatcctccctggctgcatctgtaggagacagagtcaccatcacttgccgggcaagtcagttcat
tagcagctatttacattggtatcagcaaagaccaggcaaggcccctaaactcctgatgtatgctgcctccactttgcaaagtggggtcccatca
aggttcagtggcagtggatctgggacagatttcactctcaccatcagcagtctgcaacctgaagattttgcaacttactactgtcaacagagtta
cactaacccatacacttttggccaggggaccaagaggagatcaaacgaactgtggctgcaccatctgtcttcatcttcccgccatctgatga
gcagttgaaatctggaactgcctctgttgtgtgcctgctgaataacttctatcccagagaggccaaagtacagtggaaggtggataacgccct
ccaatcgggtaactcccaggagagtgtcacagagcaggacagcaaggacagcacctacagcctcagcagcaccctgacgctgagcaaa
gcagactacgagaaacacaaagtctacgcctgcgaagtcacccatcagggcctgagctcgcccgtcacaaagagcttcaacaggggaga
gtgt
Clone No 829:
gacatccagatgacccagtctccatcctccctatctgcatctgtaggagacagagtcaccatcacttgccgggcaagtcagagca
ttgccagctatttaaattggtatcagcagaaaccagggaaagcccccaaactcctgatctatgctgcatccagtttgcatagtggggtcccatc
aagattcagtggcagtggatctgggacagatttcactctcaccatcagcagtctgcaacctgaagattttgcaacttactactgtcaacacagtt
acagtactcgattcactttcggccctgggaccaaagtggatgtcaaacgaactgtggctgcaccatctgtcttcatcttcccgccatctgatga
gcagttgaaatctggaactgcctctgttgtgtgcctgctgaataacttctatcccagagaggccaaagtacagtggaaggtggataacgccct
ccaatcgggtaactcccaggagagtgtcacagagcaggacagcaaggacagcacctacagcctcagcagcaccctgacgctgagcaaa
gcagactacgagaaacacaaagtctacgcctgcgaagtcacccatcagggcctgagctcgcccgtcacaaagagcttcaacaggggaga
gtgt
Clone No 830:
gacatccagatgacccagtctccttcgaccctgtctgcatctgtaggagacagagtcaccatcacttgccgggccagtcagagtg
ttactagtgagttggcctggtatcagcagaaaccagggaaagcccctaacttcctgatctataaggcgtctagtttagaaagtggggtcccatc
aaggttcagcggcagtggatctgggacagaattcactctcaccatcagcagcctgcagcctgatgattttgcaacttattactgccaacagtat
aatagttttccgtacacttttggccaggggaccaagctggagatcaaacgaactgtggctgcaccatctgtcttcatcttcccgccatctgatga
gcagttgaaatctggaactgcctctgttgtgtgcctgctgaataacttctatcccagagaggccaaagtacagtggaaggtggataacgccct
ccaatcgggtaactcccaggagagtgtcacagagcaggacagcaaggacagcacctacagcctcagcagcaccctgacgctgagcaaa
gcagactacgagaaacacaaagtctacgcctgcgaagtcacccatcagggcctgagctcgcccgtcacaaagagcttcaacaggggaga
gtgt
Clone No 831:
gacatccagatgacccagtctccttccaccctgtctgcatctgtaggcgacagactcaccatcacttgccgggccagtcagaatat
ttataactggttggcctggtatcagcagaaaccagggaaagcccctaaactcctgatctatgacgcctccactttggaaagtggggtcccatc
aaggttcagcggcagtggatctgggacagagttcactctcaccatcagcagcctgcagcctgatgattttgcgacttattactgccaacaatat
aatagtttgtctccgacgttcggccaagggaccaaggtggaaatcaagcgaactgtggctgcaccatctgtcttcatcttcccgccatctgatg
agcagttgaaatctggaactgcctctgttgtgtgcctgctgaataacttctatcccagagaggccaaagtacagtggaaggtggataacgccc
tccaatcgggtaactcccaggagagtgtcacagagcaggacagcaaggacagcacctacagcctcagcagcaccctgacgctgagcaa
agcagactacgagaaacacaaagtctacgcctgcgaagtcacccatcagggcctgagctcgcccgtcacaaagagcttcaacaggggag
agtgt
Clone No 835:
gacatccagttgacccagtctccatccttcctgtctgcatctttagaagacagagtcactatcacttgccgggccagtcagggcatt
agcagttatttagcctggtatcagcaaaaaccagggaaagcccctaagctcctgctcgatgctgcatccactttgcaaagtggggtcccatca
aggttcagcggcagtggatctgggacagagttcactctcacaatcagcagcctgcagcctgaagattttgcaacttattactgtcaacagctta
atagttaccctcggacgttcggccaagggaccaaggtggacatcaaacgaactgtggctgcaccatctgtcttcatcttcccgccatctgatg
agcagttgaaatctggaactgcctctgttgtgtgcctgctgaataacttctatcccagagaggccaaagtacagtggaaggtggataacgccc
tccaatcgggtaactcccaggagagtgtcacagagcaggacagcaaggacagcacctacagcctcagcagcaccctgacgctgagcaa
agcagactacgagaaacacaaagtctacgcctgcgaagtcacccatcagggcctgagctcgcccgtcacaaagagcttcaacaggggag
agtgt
Clone No 838:
gacatccagatgacccagtctccatcctccctgtctgcatctgtaggagacagagtcagcatcacttgccgggcgagtcagggc
attagcaattatttagcctggtatcagcagaaaccagggaaggttcctaagctcctgatctatgctgcatccactttgcaatcaggggtcccatc
tcggttcagtggcagtggatctgggacagatttcactctcaccatcagcagcctgcagcctgaggatgttgcaacttattactgtcaaaagtat
aacagtgcccctcaaacgttcggccaagggaccaaggtggaaatcaaacgaactgtggctgcaccatctgtcttcatcttcccgccatctgat
gagcagttgaaatctggaactgcctctgttgtgtgcctgctgaataacttctatcccagagaggccaaagtacagtggaaggtggataacgcc
ctccaatcgggtaactcccaggagagtgtcacagagcaggacagcaaggacagcacctacagcctcagcagcaccctgacgctgagca
aagcagactacgagaaacacaaagtctacgcctgcgaagtcacccatcagggcctgagctcgcccgtcacaaagagcttcaacagggga
gagtgt
Clone No 841:
gacatcgtgatgacccagtctccagactccctggctgtgtctctgggcgagagggccaccatcaactgcaggtccagccagagt
gttttatacagctccaacaataagaactacttagcttggtaccagcagaaaccaggacagcctcctaagagctcgtttactgggcatcaaccc
gggcatccggggtccctgaccgattcagtggcagcgggtctgggacagatttcactctcaccctcagcagcctgcaggctgaagatgtggc
agtttattactgtcagcagtttcatagtactcctcggacgttcggccaagggaccaaggtggagatcaaacgaactgtggctgcaccatctgtc
ttcatcttcccgccatctgatgagcagttgaaatctggaactgcctctgttgtgtgcctgctgaataacttctatcccagagaggccaaagtaca
gtggaaggtggataacgccctccaatcgggtaactcccaggagagtgtcacagagcaggacagcaaggacagcacctacagcctcagc
agcaccctgacgctgagcaaagcagactacgagaaacacaaagtctacgcctgcgaagtcacccatcagggcctgagctcgcccgtcac
aaagagcttcaacaggggagagtgt
Clone No 853:
gaaattgtgttgacgcagtctccaggcaccctgtctttgtctccaggggaaagagccaccctctcctgcagggccagtcagagtg
ttagcagcaactacttagcctggtaccagcagaaacctggccaggctcccaggctcctcatctatggtgcatccagcagggccgctggcat
gccagacaggttcagtggcagtgggtctgggacagacttcactctcaccatcagcagactggagcctgaagattttgcagtgtattactgtca
gcagtatggtaactcaccgctcactttcggcggagggaccgaggtggagatcaaacgaactgtggctgcaccatctgtcttcatcttcccgc
catctgatgagcagttgaaatctggaactgcctctgttgtgtgcctgctgaataacttctatcccagagaggccaaagtacagtggaaggtgg
ataacgccctccaatcgggtaactcccaggagagtgtcacagagcaggacagcaaggacagcacctacagcctcagcagcaccctgacg
ctgagcaaagcagactacgagaaacacaaagtctacgcctgcgaagtcacccatcagggcctgagctcgcccgtcacaaagagcttcaac
aggggagagtgt
Clone No 855:
gacatccagatgacccagtctccatcttctgtgtctgcatctgtaggagacagagtcaccatcacttgtcgggcgagtcaggctatt
agtaactggttagcctggtatcagcagaaaccaggaaaagcccctaagctcctgatctatgctgcatccagtttgcaaagtggggtcccatca
agattcagcggcagtggatctgggacagatttcactctcactatcagcggcctgcagcctgaggattttgcaacttactattgtcaacaggctg
acactttccctttcactttcggccctgggaccaaagtggatatcaaacgaactgtggctgcaccatctgtcttcatcttcccgccatctgatgag
cagttgaaatctggaactgcctctgttgtgtgcctgctgaataacttctatcccagagaggccaaagtacagtggaaggtggataacgccctc
caatcgggtaactcccaggagagtgtcacagagcaggacagcaaggacagcacctacagcctcagcagcaccctgacgctgagcaaag
cagactacgagaaacacaaagtctacgcctgcgaagtcacccatcagggcctgagctcgcccgtcacaaagagcttcaacaggggagag
tgt
Clone No 856:
gatattgtgatgacccagactccactctccctgcccgtcacccctggagagccggcctccatctcctgcaggtctagtcagagcct
cttggatagtaatgatggaaacacctatttggactggtacctgcagaagccagggcagtctccacagctcctgatttatacattttcctatcggg
cctctggagtcccagacaggttcagtggcagtgggtctggcactgatttcacactgaaaatcagcagggtggaggccgaggatgttggagt
ttattactgcatgcaacgtatcgagtttccgtacacttttggccaggggaccaagctggagatcaaacgaactgtggctgcaccatctgtcttca
tcttcccgccatctgatgagcagttgaaatctggaactgcctctgttgtgtgcctgctgaataacttctatcccagagaggccaaagtacagtg
gaaggtggataacgccctccaatcgggtaactcccaggagagtgtcacagagcaggacagcaaggacagcacctacagcctcagcagc
accctgacgctgagcaaagcagactacgagaaacacaaagtctacgcctgcgaagtcacccatcagggcctgagctcgcccgtcacaaa
gagcttcaacaggggagagtgt
Clone No 857:
gatattgtgatgacccagtctccactctccctgcccgtcacccctggagagccggcctccatctcctgcaggtctagtcagagcct
cctgcatagaaatgagtacaactatttggattggtacttgcagaagccagggcagtctccacagctcctgatctattggggttctaatcgggcc
tccggggtccctgacaggttcagtggcagtggatcaggcacagattttacactgaaaatcagcagagtggaggctgaggatgttggggttta
ttactgcatgcaaactctacaaactcctcggacgttcggccaagggaccaaggtggaaatcaaacgaactgtggctgcaccatctgtcttcat
cttcccgccatctgatgagcagttgaaatctggaactgcctctgttgtgtgcctgctgaataacttctatcccagagaggccaaagtacagtgg
aaggtggataacgccctccaatcgggtaactcccaggagagtgtcacagagcaggacagcaaggacagcacctacagcctcagcagca
ccctgacgctgagcaaagcagactacgagaaacacaaagtctacgcctgcgaagtcacccatcagggcctgagctcgcccgtcacaaag
agcttcaacaggggagagtgt
Clone No 858:
gacatccagatgacccagtctccatcctccgtgtctgcatctgtgggagacagagtcaccatcacttgccaggcgagtcaagaca
ttagcaactatttaaattggtatcagcagaaaccagggaaagcccctaagctcctgatcttcgatgcaaccaaattggagacaggggtcccaa
caaggttcattggaagtggatctgggacagattttactgtcaccatcaccagcctgcagcctgaagatgttgcaacatattactgtcaacacttt
gctaatctcccatacacttttggccaggggaccaagctggagatcaagcgaactgtggctgcaccatctgtcttcatcttcccgccatctgatg
agcagttgaaatctggaactgcctctgttgtgtgcctgctgaataacttctatcccagagaggccaaagtacagtggaaggtggataacgccc
tccaatcgggtaactcccaggagagtgtcacagagcaggacagcaaggacagcacctacagcctcagcagcaccctgacgctgagcaa
agcagactacgagaaacacaaagtctacgcctgcgaagtcacccatcagggcctgagctcgcccgtcacaaagagcttcaacaggggag
agtgt
Clone No 859:
gacatccagatgacccagtctccatcttccctgtctgcatctgtaggagacagagtcaccatcacttgccgggcgagtcagggca
ttaggaattatttagcctggtatcagcagaaaccagggaaagttcctaagctcctggtctttgctgcatccactttgcaatcaggggtcccatct
cggttcagtggcagtggatctgggacagatttcactctcaccatcagcagcctgcagcctgaggatgttgcaacttattactgtcaaaggtata
acagtgccccgctcactttcggcggagggacgaaggtggagatcaaacgaactgtggctgcaccatctgtcttcatcttcccgccatctgat
gagcagttgaaatctggaactgcctctgttgtgtgcctgctgaataacttctatcccagagaggccaaagtacagtggaaggtggataacgcc
ctccaatcgggtaactcccaggagagtgtcacagagcaggacagcaaggacagcacctacagcctcagcagcaccctgacgctgagca
aagcagactacgagaaacacaaagtctacgcctgcgaagtcacccatcagggcctgagctcgcccgtcacaaagagcttcaacagggga
gagtgt
Clone No 861:
gacatccagatgacccagtctccatcctccctgtctgcatctgtaggagacagagtcaccatcacttgccgggcaagtcagatcat
tgccagctatttaaattggtatcagcagaaaccaggcagagcccctaagctcctgatctatgctgcatccagtttgcaaagtggggtcccatca
aggttcagtggcagtggatctgggacagatttcactctcaccatcagcagtctgcaacctgaagattttgcaacttactactgtcaacagagtta
cagtacccccatattcactttcggccctgggaccaaggtgaatatcaaacgaactgtggctgcaccatctgtcttcatcttcccgccatctgatg
agcagttgaaatctggaactgcctctgttgtgtgcctgctgaataacttctatcccagagaggccaaagtacagtggaaggtggataacgccc
tccaatcgggtaactcccaggagagtgtcacagagcaggacagcaaggacagcacctacagcctcagcagcaccctgacgctgagcaa
agcagactacgagaaacacaaagtctacgcctgcgaagtcacccatcagggcctgagctcgcccgtcacaaagagcttcaacaggggag
agtgt
Clone No 863:
gaaattgtgttgacacagtctccagccaccctgtctttgtctccaggggaaagagccaccctctcctgcaggaccagtcagagtgt
tagcagctacttagcctggtaccaacagaaacctggccaggctcccaggctcctcatctatgatgcttccaatagggccactggcatcccag
ccaggttcagtggcagtgggtagggacagacttcactctcaccatcagcagcctagagcctgaagattttgcagtttattactgtcagcagc
gtagtgactggctcactttcggcggagggaccaaggtggagatcaaacgaactgtggctgcaccatctgtcttcatcttcccgccatctgatg
agcagttgaaatctggaactgcctctgttgtgtgcctgctgaataacttctatcccagagaggccaaagtacagtggaaggtggataacgccc
tccaatcgggtaactcccaggagagtgtcacagagcaggacagcaaggacagcacctacagcctcagcagcaccctgacgctgagcaa
agcagactacgagaaacacaaagtctacgcctgcgaagtcacccatcagggcctgagctcgcccgtcacaaagagcttcaacaggggag
agtgt
Clone No 868:
gaaattgtaatgacacagtctccagccaccctgtctgtgtctccaggggaaagagccaccctctcctgcagggccagtcagagta
ttaaaaacaacttggcctggtaccaggtgaaacctggccaggctcccaggctcctcacctctggtgcatccgccagggccactggaattcca
ggcaggttcagtggcagtgggtctgggactgacttcactctcaccatcagcagcctccagtctgaagatattgcagtttattactgtcaggagt
ataataattggcccctgctcactttcggcggagggaccaaggtggagatccaacgaactgtggctgcaccatctgtcttcatcttcccgccat
ctgatgagcagttgaaatctggaactgcctctgttgtgtgcctgctgaataacttctatcccagagaggccaaagtacagtggaaggtggata
acgccctccaatcgggtaactcccaggagagtgtcacagagcaggacagcaaggacagcacctacagcctcagcagcaccctgacgct
gagcaaagcagactacgagaaacacaaagtctacgcctgcgaagtcacccatcagggcctgagctcgcccgtcacaaagagcttcaaca
ggggagagtgt
Clone No 870:
gacatccagatgacccagtctcctccctccctgtctgcatctgtgggagacagagtcaccatcacttgccgggcaagtcagagga
ttgccagctatttaaattggtatcagcagaaaccagggagagcccctaagctcctgatctttgctgcatccagtttacaaagtggggtcccatc
aaggttcagtggcagtggatctgggacagacttcactctcaccatcagtagtctgcaacctgaagattatgcgacttactactgtcaacagagt
tacagtactcccatctacacttttggccaggggaccaagctggagatcaaacgaactgtggctgcaccatctgtcttcatcttcccgccatctg
atgagcagttgaaatctggaactgcctctgttgtgtgcctgctgaataacttctatcccagagaggccaaagtacagtggaaggtggataacg
ccctccaatcgggtaactcccaggagagtgtcacagagcaggacagcaaggacagcacctacagcctcagcagcaccctgacgctgag
caaagcagactacgagaaacacaaagtctacgcctgcgaagtcacccatcagggcctgagctcgcccgtcacaaagagcttcaacaggg
gagagtgt
Clone No 871:
gacatccagatgacccagtctccatcctccctgtctgcatctgtaggagacagagtcaccatcacttgccaggcgagtcagggca
ttagcaactatttaaattggtatcaacagaaaccagggaaagcccctaagctcctgatcttcgatgcatccaatttggaatcagaggtcccatc
aaggttcagtggacgtggatctgggacagattttactttctccatcagcagcctgcagcctgaagatattgcaacatatttctgtcaacagtatg
ataatttcccgtacacttttggccaggggaccaagctggagatcaaacgaactgtggctgcaccatctgtcttcatcttcccgccatctgatga
gcagttgaaatctggaactgcctctgttgtgtgcctgctgaataacttctatcccagagaggccaaagtacagtggaaggtggataacgccct
ccaatcgggtaactcccaggagagtgtcacagagcaggacagcaaggacagcacctacagcctcagcagcaccctgacgctgagcaaa
gcagactacgagaaacacaaagtctacgcctgcgaagtcacccatcagggcctgagctcgcccgtcacaaagagcttcaacaggggaga
gtgt
Clone No 880:
gacatccagatgacccagtctccatcctccctggctgcatctgtaggagacagagtcaccatcacctgccgggcaagtcagacg
attgccagttatgtaaattggtatcaacagaaaccagggaaagcccctaatctcctgatctatgctgcatccagtttgcaaagtggggtcccat
caaggttcagtggcagtggatctgggacagatttcactctcaccatcagcagtctgcaacctgaagattttgcatcttacttctgtcaacagagt
tacagtttcccgtacacttttggccaggggaccaagctggatatcaaacgaactgtggctgcaccatctgtcttcatcttcccgccatctgatga
gcagttgaaatctggaactgcctctgttgtgtgcctgctgaataacttctatcccagagaggccaaagtacagtggaaggtggataacgccct
ccaatcgggtaactcccaggagagtgtcacagagcaggacagcaaggacagcacctacagcctcagcagcaccctgacgctgagcaaa
gcagactacgagaaacacaaagtctacgcctgcgaagtcacccatcagggcctgagctcgcccgtcacaaagagcttcaacaggggaga
gtgt
Clone No 881:
gacatccagatgacccagtctccatcctccctgtctgcatctgtaggagacagagtcaccatcacttgccgggcaagtcagacca
ttgccagctatgtaaattggtatcagcagaaaccagggaaagcccctaagctcctgatctatgctgcatccaatttgcaaagtggggtcccttc
aaggttcagtggcagtggatctgggacagatttcactctcaccatcagcagtctgcaacctgaagattttgcaacttactactgtcaacagagtt
acagtgtccctcggctcactttcggcggagggaccaaggtggacatcacacgaactgtggctgcaccatctgtcttcatcttcccgccatctg
atgagcagttgaaatctggaactgcctctgttgtgtgcctgctgaataacttctatcccagagaggccaaagtacagtggaaggtggataacg
ccctccaatcgggtaactcccaggagagtgtcacagagcaggacagcaaggacagcacctacagcctcagcagcaccctgacgctgag
caaagcagactacgagaaacacaaagtctacgcctgcgaagtcacccatcagggcctgagctcgcccgtcacaaagagcttcaacaggg
gagagtgt
Clone No 884:
gacatccagatgacccagtctccatcctccctgtctgcatctgtaggagacagagtcaccatcacttgccggtcaagtcagaccat
tagcgtctttttaaattggtatcagcagaaaccagggaaagcccctaagctcctgatctatgccgcatccagtttgcacagtgcggtcccatca
aggttcagtggcagtggatctgggacagatttcactctcaccatcagcagtctgcaacctgaagattctgcaacttactactgtcaagagagttt
cagtagctcaactttcggcggagggaccaaggtggagatcaaacgaactgtggctgcaccatctgtcttcatcttcccgccatctgatgagc
agttgaaatctggaactgcctctgttgtgtgcctgctgaataacttctatcccagagaggccaaagtacagtggaaggtggataacgccctcc
aatcgggtaactcccaggagagtgtcacagagcaggacagcaaggacagcacctacagcctcagcagcaccctgacgctgagcaaagc
agactacgagaaacacaaagtctacgcctgcgaagtcacccatcagggcctgagctcgcccgtcacaaagagatcaacaggggagagt
gt
Clone No 886:
gaaattgtaatgacacagtctccagccaccctgtctgtgtctccaggggaaacagccaccctctcctgcagggccagtcagagtg
ttagcagcaacttagcctggtaccaacataaacctggccaggctcccaggctcctcatccatagtgcatccaccagggccactgggatccca
gccaggttcagtggcagtgggtctgggacagagttcactctcaccataagcagcctgcagtctgaagattttgcagtttattactgtcagcagt
ataatatgtggcctccctggacgttcggccaagggaccaaggtggaaatcaaacgaactgtggctgcaccatctgtcttcatcttcccgccat
ctgatgagcagttgaaatctggaactgcctctgttgtgtgcctgctgaataacttctatcccagagaggccaaagtacagtggaaggtggata
acgccctccaatcgggtaactcccaggagagtgtcacagagcaggacagcaaggacagcacctacagcctcagcagcaccctgacgct
gagcaaagcagactacgagaaacacaaagtctacgcctgcgaagtcacccatcagggcctgagctcgcccgtcacaaagagatcaaca
ggggagagtgt
Clone No 888:
gatattgtgatgacccagtctccactctccctgcccgtcacccctggagcgccggcctccatctcctgcaggtctagtcagagcct
cctgcgtactaatggatacaactatttggattggtacctgcagaagccagggcagtctccacagctcctgatctatttgggttctattcgggcct
ccggggtccctgacaggttcagtggcagtggctcaggcacagattttacactgaaaatcagcagagtggaggctgaggatgttggggtttat
tactgcatgcaatctctacaaacttcgatcaccttcggccaagggacacgactggagattaaacgaactgtggctgcaccatctgtcttcatctt
cccgccatctgatgagcagttgaaatctggaactgcctctgttgtgtgcctgctgaataacttctatcccagagaggccaaagtacagtggaa
ggtggataacgccctccaatcgggtaactcccaggagagtgtcacagagcaggacagcaaggacagcacctacagcctcagcagcacc
ctgacgctgagcaaagcagactacgagaaacacaaagtctacgcctgcgaagtcacccatcagggcctgagctcgcccgtcacaaagag
cttcaacaggggagagtgt
Clone No 894:
gaaattgtaatgacacagtctccagccaccctgtctgtgtctccgggggaaagagccaccctctcctgcagggctagtcagagtg
ttggcaacaacttagcctggtaccagcagagacctggccaggctcccagactcctcatctatggtgcgtccaccagggccactggtatccca
gccaggttcagtggcagtgggtctgggacagagttcactctcaccatcagcagcctgcagtctgaggattttgcagtttattactgtcagcagt
atgataagtggcctgagacgttcggccaggggaccaaggtggacatcaagcgaactgtggctgcaccatctgtcttcatcttcccgccatct
gatgagcagttgaaatctggaactgcctctgttgtgtgcctgctgaataacttctatcccagagaggccaaagtacagtggaaggtggataac
gccctccaatcgggtaactcccaggagagtgtcacagagcaggacagcaaggacagcacctacagcctcagcagcaccctgacgctga
gcaaagcagactacgagaaacacaaagtctacgcctgcgaagtcacccatcagggcctgagctcgcccgtcacaaagagcttcaacagg
ggagagtgt

(0161J In all of the above-discussed 44 clones, the encoded antibodies include the same constant IgG heavy chain, which has the following amino acid sequence (SEQ ID NO: 178):

SASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRVE
PKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSR
WQQGNVFSCSVMHEALHNHYTQKSLSLSPGK

The genomic sequence encoding this heavy chain has the following nucleic acid sequence (SEQ ID NO: 177):

agtgcctccaccaagggcccatcggtcttccccctggcaccctcctccaagagcacctctgggggcacagcggccctgggctg
cctggtcaaggactacttccccgaaccggtgacggtgtcgtggaactcaggcgccctgaccagcggcgtgcacaccttcccgg
ctgtcctacagtcctcaggactctactccctcagcagcgtggtgaccgtgccctccagcagcttgggcacccagacctacatctg
caacgtgaatcacaagcccagcaacaccaaggtggacaagagagttggtgagaggccagcacagggagggagggtgtctgc
tggaagccaggctcagcgctcctgcctggacgcatcccggctatgcagtcccagtccagggcagcaaggcaggccccgtctg
cctcttcacccggaggcctctgcccgccccactcatgctcagggagagggtcttctggctttttccccaggctctgggcaggcac
aggctaggtgcccctaacccaggccctgcacacaaaggggcaggtgctgggctcagacctgccaagagccatatccgggag
gaccctgcccctgacctaagcccaccccaaaggccaaactctccactccctcagctcggacaccttctctcctcccagattccag
taactcccaatcttctctctgcagagcccaaatcttgtgacaaaactcacacatgcccaccgtgcccaggtaagccagcccaggc
ctcgccctccagctcaaggcgggacaggtgccctagagtagcctgcatccagggacaggccccagccgggtgctgacacgtc
cacctccatctcttcctcagcacctgaactcctggggggaccgtcagtcttcctcttccccccaaaacccaaggacaccctcatga
tctcccggacccctgaggtcacatgcgtggtggtggacgtgagccacgaagaccctgaggtcaagttcaactggtacgtggac
ggcgtggaggtgcataatgccaagacaaagccgcgggaggagcagtacaacagcacgtaccgtgtggtcagcgtcctcacc
gtcctgcaccaggactggctgaatggcaaggagtacaagtgcaaggtctccaacaaagccctcccagcccccatcgagaaaa
ccatctccaaagccaaaggtgggacccgtggggtgcgagggccacatggacagaggccggctcggcccaccctctgccctg
agagtgaccgctgtaccaacctctgtccctacagggcagccccgagaaccacaggtgtacaccctgcccccatcccgggagg
agatgaccaagaaccaggtcagcctgacctgcctggtcaaaggcttctatcccagcgacatcgccgtggagtgggagagcaat
gggcagccggagaacaactacaagaccacgcctcccgtgctggactccgacggctccttcttcctctatagcaagctcaccgtg
gacaagagcaggtggcagcaggggaacgtcttctcatgctccgtgatgcatgaggctctgcacaaccactacacgcagaagag
cctctccctgtccccgggtaaatga

In this sequence exons are indicated by double underlining. Further, the initial Ser-encoding nucleotides agt (bold underline) are created as a consequence of the introduction into the Xhol digested expression vector of an XhoI digested PCR product encoding the variable heavy chain site in the IgG expression vector.

The above-discussed V_{H} and V_{L} coding pairs were selected according to the binding specificity to various antigens and peptides in ELISA and/or FLISA, epitope mapping, antigen diversity, and sequence diversity. The selected cognate V-gene pairs were subjected to clone repair if errors were identified.

### Example 2

### Functional in vitro Testing of Mono- and Polyclonal Anti-RSV Antibodies

In vitro neutralization experiments have been performed both with single antibody clones and with combinations of purified antibodies. All the antibody mixtures described below are constituted of a number of individual anti-RSV antibodies of the present invention, which were combined into a mixture using equal amounts of the different antibodies.

Preparation of Live RSV for in vitro use

Human laryngeal epithelial HEp-2 cells (ATCC CLL-23) were seeded in 175 cm² flasks at 1x10⁷ cells/flask. The cells were infected with either the RSV Long (ATCC number VR-26), the RSV A2 (Advanced Biotechnologies Inc., ATCC number VR-1540) the RSV B1 (ATCC number VR-1400) or the RSV B Wash/18537 (Advanced Biotechnologies Inc., ATCC number VR-1580) strain in 3 ml serum-free medium at a ratio of 0.1 pfu/cell. Cells were infected for 2 h at 37° C.; 5% CO₂ followed by addition of 37 ml of complete MEM medium. Cells were incubated until cytopathic effects were visible. The cells were detached by scraping and the media and cells were sonicated for 20 sec and aliquoted, snap frozen in liquid nitrogen and stored at −80° C. Plaque reduction neutralization test (PRNT)

HEp-2 cells were seeded in 96-well culture plates at 2×10⁴ cells/well, and incubated overnight at 37° C.; 5% CO₂. The test substances were diluted in serum-free MEM and allowed to pre-incubate with RSV in the absence or presence of complement (Complement sera from rabbit, Sigma) for 30 min at 37° C. This mixture was applied to the monolayer of HEp-2 cells and incubated for 24-72 h at 37° C.; 5% CO₂. The cells were fixed with 80% acetone; 20% PBS for 20 min. After washing, biotinylated goat anti-RS V antibody (AbD Serotec) was added (1:200) in PBS with 1% BSA and incubated for 1 h at room temperature. After washing, HRP-avidin was added and allowed to incubate for 30 min. Plaques were developed by incubation with 3-amino-9-ethylcarbazole (AEC) substrate until plaques were visible by microscopy, e.g., for 25 min (RSV Long) or 45 min (RSV B1). Plaques were counted in a Bioreader (Bio-Sys GmbH). EC₅₀ values (effective concentrations required to induce a 50% reduction in the number of plaques) were calculated where applicable to allow for a comparison of the potencies.

### Testing of Single Antibodies

The neutralizing activity of each antibody was determined in the presence of complement against RSV subtype A and B strains. The EC₅₀ values of a number of the purified antibodies are shown in Table 5. Blank fields indicate that the analysis has not been performed yet. ND indicates that an EC₅₀ value could not be determined in the PRNT due to a very low or lacking neutralizing activity.

TABLE 5
EC₅₀ values of purified anti-RSV protein F and protein
G antibodies against RSV subtype A and B.

| | | EC₅₀ value (μg/ml) | | | |
|---|---|---|---|---|---|
| Clone | Antigen-specificity | Long | A2 | 18537 | B1 |
| 793 | G | 2.52 | | 0.09 | |
| 800 | F | 0.15 | | | 0.16 |
| 810 | F | 0.04-0.06 | 0.02 | 0.02-0.14 | 0.29 |
| 816 | G | ND | | ND | |
| 818 | F | 0.15 | | 0.21 | |
| 819 | F | 0.18 | | 0.09 | |
| 824 | F | 0.03 | 0.007 | 0.02 | 0.07 |
| 825 | F | 0.12 | | 0.04 | |
| 827 | F | 0.16 | | 0.10 | |
| 831 | F | 0.08 | | 0.72 | 1.66 |
| 853 | G | 0.13* | | 0.14 | |
| 855 | G | 6.35 | | ND | |
| 856 | G | ND | | ND | |
| 858 | F | ND | | 0.13 | |
| 868 | G | ND | | | |
| 880 | F | 0.38 | | 0.95 | 0.40 |
| 888 | G | 0.14 | | | |
| 894 | F | 0.08 | | 0.07 | |
| Palivizumab | F | 0.14 | 0.15 | 0.20 | |

### Mixtures of Anti-F Antibodies

The ability of mixtures of anti-RSV protein F antibodies to neutralize RSV strains of subtype A and B was compared with the neutralizing effect obtained using Palivizumab (also an anti-F antibody). The neutralization capability was assessed using a microneutralization test or the PRNT. In an initial experiment two antibody mixtures, anti-F(I) and anti-F(II), containing five and eleven distinct anti-F antibodies, respectively were compared against Palivizumab using the microneutralizating test. Anti-F(I) is composed of antibodies obtained from clones 810, 818, 819, 825 and 827. Anti-F(II) is composed of antibodies obtained from clones 735, 800, 810, 818, 819, 825, 827, 863, 880, 884 and 894. Both composition Anti-F(I) and F(II) were more potent than Palivizumab with respect to neutralization of RSV strains of both subtypes.

Both the in vitro assays and the combinations of clones have been refined since this initial experiment and a number of combinations of F-specific antibody clones that are highly potent in the presence of complement have been identified. The neutralizing potencies, expressed as EC₅₀ values (effective concentrations required to induce a 50% reduction in the number of plaques), of additional anti-F antibody compositions are listed in Table 6. Irrespective of the exact number of clones in the compositions, the majority of the tested combinations of F-specific antibodies were more potent than Palivizumab with respect to neutralization of RSV strain subtype A.

### Mixtures of Anti-G Antibodies

The ability of mixtures of anti-G antibodies to neutralize RSV strains of subtype A was tested using the PRNT. The EC₅₀ values from the tested anti-G antibody compositions are listed in Table 6. Most of the compositions of two anti-G antibodies did not exhibit a markedly increased ability to neutralize virus compared to the individual anti-G antibodies. Some combinations of two or three anti-G antibodies never reached 100% neutralization of the virus, irrespective of the concentration. However, when additional anti-G antibodies were added to the composition the potency increased, possibly indicating a synergistic neutralizing effect between the anti-G antibodies.

### Mixtures of Anti-F and Anti-G Antibodies

The ability of mixtures of anti-RSV protein F and protein G antibodies to neutralize RSV subtype B strain was compared with the neutralizing effect obtained using Palivizumab.

Initially, the neutralizing activity of two antibody mixtures, anti-F(I)G and anti-F(II)G, was measured in the microneutralization fusion inhibition assay. Each of these mixtures contains the anti-F antibodies of composition anti-F(I) and anti-F(II) described above as well as anti-G antibodies obtained from clones 793, 796, 838, 841, 856 and 888. Both composition Anti-F(I)G and F(II)G were more potent than Palivizumab with respect to neutralization of the RSV B1 strain. Further, the neutralizing activity of the two mixtures was more or less equal.

A large number of different combinations of both anti-F and anti-G antibodies have been tested in the PRNT in the presence or absence of complement. EC₅₀ values obtained by this assay in the presence of active complement are presented in Table 6. All of the tested compositions including both anti-F and anti-G antibodies do neutralize RSV subtype A and the majority of these are more potent than Palivizumab.

TABLE 6
EC₅₀ values of combinations of anti-RSV antibodies against RSV subtype A and B.

| Composition | | EC50 value (μg/ml) | | | |
|---|---|---|---|---|---|
| number | Antibodies in composition | Long | A2 | 18537 | B1 |
| 1 | 810, 818, 819, 825, 827 | 0.19 | | | 0.38 |
| 2 | 810, 818, 819, 825, 827, 831, 858, | 0.34 | | | |
| | 863, 884, 894, 793, 796, 816, 838, | | | | |
| | 853, 856, 859, 888 | | | | |
| 3 | 810, 818, 825, 827, 884, 886, 793, | 0.30 | | | |
| | 853, 868, 888 | | | | |
| 4 | 810, 818, 825, 827, 831, 858, 884, | 0.19 | | | |
| | 886, 793, 796, 816, 853, 856, 868, | | | | |
| | 888 | | | | |
| 5 | 810, 818, 825, 827, 831, 858, 884, | 0.21 | | | |
| | 886, 793, 853, 868, 888 | | | | |
| 6 | 810, 819, 825, 827, 831, 793, 853, | 0.20 | | | |
| | 856, 858, 868 | | | | |
| 7 | 810, 811, 817, 819, 825, 827, 831, | 0.18 | | | |
| | 838, 853, 856, 858, 859, 863, 868 | | | | |
| 8 | 800, 801, 811, 838, 853, 855, 859, | 0.92 | | | |
| | 861, 880, 894, 736, 795, 796, 799 | | | | |
| 9 | 810, 818, 825 | 0.14 | | 0.03 | 0.29 |
| 10 | 810, 818, 819, 825, 827, 884 | 0.21 | | | 0.42 |
| 11 | 810, 818, 819, 825, 827, 884, 886 | 0.15 | | | 0.29 |
| 12 | 793, 816, 853, 856 | 0.06 | | | |
| 13 | 793, 816, 853, 855, 856 | 0.03 | 0.03 | 0.86 | |
| 14 | 793, 868, 888, 853, 856 | 0.34 | | | |
| 15 | 793, 796, 818, 816, 838, 853, 855, | 0.11 | | | |
| | 856, 859, 868, 888 | | | | |
| 16 | 810, 818, 827 | 0.11 | | | 0.21 |
| 17 | 810, 818, 825, 827, 858, 886 | 0.10 | | 0.05 | 0.16 |
| 18 | 810, 818, 825, 827, 858, 886, 793, | 0.04 | | 0.06 | 0.15 |
| | 816, 853, 855, 856 | | | | |
| 19 | 818, 825, 827, 858, 886, 793, 816, | 0.06 | | | |
| | 853, 855, 856 | | | | |
| 20 | 810, 818, 819, 825, 827, 858, 793, | 0.10 | | 0.06 | |
| | 816, 853, 855, 856 | | | | |
| 21 | 810, 793, 816, 853, 855, 856 | 0.04 | | | |
| 22 | 818, 825, 827, 831, 858, 886, 793, | 0.06 | | | |
| | 816, 853, 855, 856 | | | | |
| 23 | 818, 825, 827, 831, 858, 819, 793, | 0.06 | | 0.03 | |
| | 816, 853, 855, 856 | | | | |
| 24 | 818, 827, 831, 858, 819, 793, 816, | 0.06 | | 0.04 | |
| | 853, 855, 856 | | | | |
| 25 | 810, 818, 819, 824, 825, 827, 858, | 0.07 | | | |
| | 793, 816, 853, 855, 856 | | | | |
| 26 | 831, 818, 819, 824, 825, 827, 858, | 0.08 | | | |
| | 793, 816, 853, 855, 856 | | | | |
| 27 | 831, 818, 819, 824, 827, 858, 793, | 0.05 | | | |
| | 816, 853, 855, 856 | | | | |
| 28 | 810, 818, 824 | 0.03-0.06 | 0.04 | 0.04 | 0.04 |
| 29 | 810, 824 | 0.05 | | | |
| 30 | 818, 824 | 0.04 | | | |
| 31 | 810, 818 | 0.08-0.11 | | | |
| 32 | 824, 793, 816, 853, 855, 856 | 0.05 | | | |
| 33 | 810, 818, 819, 824, 825, 827, 858, | 0.03-0.07 | 0.06 | 0.03 | 0.06 |
| | 894, 793, 816, 853, 855, 856 | | | | |
| 34 | 810, 818, 819, 824, 825, 827, 894, | 0.07 | | | |
| | 793, 816, 853, 855, 856 | | | | |
| 35 | 793, 816 | 5.94 | | | |
| 36 | 855, 856 | ND | | | |
| 37 | 793, 856 | ND | | | |
| 38 | 793, 853 | 2.35 | | | |
| 39 | 853, 856 | 0.21 | | | |
| 40 | 793, 853, 856 | 2.84 | | | |
| 41 | 793, 816, 853 | 1.97 | | | |
| 42 | 853, 855, 856 | 0.25 | | | |
| 43 | 793, 816, 853, 856 | 0.45 | | | |
| 44 | 793, 853, 855 | 0.26 | | | |
| 45 | 793, 853, 855, 856 | 0.16 | | | |
| 46 | 816, 853, 855, 856 | 0.07 | | | |
| 47 | 816, 856 | 0.06 | | | |
| 48 | 816, 853 | 0.75 | | | |
| 49 | 816, 853, 856 | 0.07 | | | |
| 50 | 810, 818, 824, 816 | 0.09 | | | |
| 51 | 810, 818, 824, 853 | 0.11 | | | |
| 52 | 810, 818, 824, 856 | 0.10 | | | |
| 53 | 810, 818, 824, 816, 853 | 0.09 | | | |
| 54 | 810, 818, 824, 816, 856 | 0.05 | | | |
| 55 | 810, 818, 824, 853, 856 | 0.08 | | | |
| 56 | 810, 818, 824, 816, 853, 856 | 0.05 | 0.03-0.05 | 0.03 | 0.06 |
| | Palivizumab (Synagis) | 0.14 | 0.15 | 0.20 | |

Blank fields indicate that the analysis has not been performed yet. ND indicates that an EC₅₀ value could not be determined in the PRNT due to a very low or lacking neutralizing activity.

### Example 3

### In vivo Testing of Poly- and Monoclonal Anti-RSV Antibodies Reduction of Viral Loads in the Lungs of RSV-Infected Mice

The in vivo protective capacity of combinations of purified antibodies of the invention against RSV infection has been demonstrated in the BALB/c mouse model (Taylor et al. 1984. Immunology 52, 137-142; Mejias, et al. 2005. Antimicrob. Agents Chemother. 49: 4700-4707).

### Mouse Challenge Model

7-8-weeks old female BALB/c mice were inoculated intraperitoneally with 0.2 ml antibody preparation on day −1 of study. Placebo treated mice were similarly inoculated i.p. with 0.1 ml PBS buffer. On day 0 of study, the mice were anesthetized using inhaled isofluorane and inoculated intranasally with 10⁻⁶-10⁻⁷ pfu of RSV strain A2 in 50 μl or with cell lysate (mock inoculum). Animals were allowed 30 seconds to aspirate the inoculum whilst held upright until fully recovered from the anaesthesia.

Five days after challenge, the mice were killed with an overdose of sodium pentobarbitone. At post-mortem, blood was obtained by exsanguination from the axillary vessels for preparation of sera. Lungs were removed and homogenized in 2.5 ml buffer with sterile sand. Lung homogenates were centrifuged to sediment sand and cell debris and supernatants were aliquoted and stored at −70° C.

The virus load was initially determined by quantification of the number of RSV RNA copies in the lung samples using reverse transcriptase (RT-) PCR. RNA was extracted from the lung homogenate samples using the MagNA Pure LC Total Nucleic Acid kit (Roche Diagnostics) automated extraction, system according to the manufacturer's instructions. Detection of RSV RNA was performed by single-tube real-time RT-PCR using the LightCycler instrument and reagents (Roche Diagnostics) with primers and fluorophore-labeled probes specific for the N gene of RSV subtype A as described by Whiley et al. (J. Clinical Microbiol. 2002, 40: 4418-22). Samples with known RSV RNA copy numbers were similarly analyzed to derive a standard ‘curve.

Subsequently, the number of RSV RNA copies in the lung samples was determined using quantitative reverse transcriptase (RT-) PCR. RNA was extracted from the lung homogenate samples using the the RNeasy mini kit (Qiagen) according to the manufacturer's instructions. Detection of RSV RNA was performed by using the SuperScript III Platinum One-Step Quantitative RT-PCR System (Invitrogen) with primers and fluorophore-labeled probes specific for the N gene of RSV subtype A as described below. Samples with known RSV RNA copy numbers were similarly analyzed to derive a standard curve.

### RSV Subtype A Specific Primers and Probe for Quantitative RT-PCR.

| Name | Sequence 5′-3′ |
|---|---|
| RSV-A forward | CAA CAA AGA TCA ACT TCT GTC ATC |
| RSV-A reverse | GCA CAT CAT AAT TAG GAG TAT CAA T |
| RSA Probe | 6-FAM-CA CCA TCC AAC GGA GCA CAG GAG |
| | AT-TAMRA |

In table 7a, data from an experiment with four different anti-RSV rpAb consisting of equal amounts of different antibody clones of the invention (described in table 6) and clone 810 alone are presented in comparison with data from uninfected control animals and placebo (PBS) treated animals of the same experiment. Each treatment group contained 5 mice and the samples were obtained on day five post-infection, which is approximately at the peak of virus replication in this model. As shown in Table 7a, the rpAb combinations effectively reduce the virus load by at least an order of magnitude when given prophylactically at 25 mg/kg of body weight. Copy numbers are presented as means±standard deviations.

TABLE 7a
Virus loads in the lungs of mice following
prophylaxis and RSV challenge.

| | Virus load by RT-PCR |
|---|---|
| Treatment group (dose) | (log10 RSV RNA copies/ng total RNA) |
| Uninfected | Negative |
| PBS | 4.11 ± 0.12 |
| Anti-RSV rpAb 18 (25 mg/kg) | 2.74 ± 0.16 |
| Anti-RSV rpAb 18 (5 mg/kg) | 3.40 ± 0.09 |
| Anti-RSV rpAb 9 (25 mg/kg) | 2.95 ± 0.19 |
| Anti-RSV rpAb 9 (5 mg/kg) | 3.56 ± 0.31 |
| Anti-RSV rpAb 17 (25 mg/kg) | 2.81 ± 0.29 |
| Anti-RSV rpAb 17 (5 mg/kg) | 3.39 ± 0.12 |
| Anti-RSV rpAb 13 (25 mg/kg) | 3.02 ± 0.33 |
| Anti-RSV rpAb 13 (5 mg/kg) | 3.34 ± 0.26 |
| Clone 810 (25 mg/kg) | 3.03 ± 0.16 |
| Clone 810 (5 mg/kg) | 3.37 ± 0.22 |

In table 7b, data from a second study with three different anti-RSV rpAb consisting of equal amounts of different antibody clones of the invention (described in table 6) and clone 824 alone are presented in comparison with data from uninfected control animals, placebo (PBS) treated animals and Palivizumab (Synagis) treated animals of the same experiment. Each treatment group contained 5 mice and the samples were obtained on day five post-infection. Copy numbers are presented as means±standard deviations.

In table 7c, data from a third study with anti-RSV rpAb 33 consisting of equal amounts of different antibody clones of the invention (described in table 6) are presented in comparison with data from uninfected control animals, placebo (PBS) treated animals and Palivizumab (Synagis) treated animals of the same experiment. Each treatment group except the last three contained 5 mice and the samples were obtained on day five post-infection. One mouse was removed from each of the groups treated with anti-RSV rpAb 33 at 15, 5 and 1.5 mg/kg body weight since it was discovered that they were never injected with antibody. Copy numbers are presented as means±standard deviations.

In all three studies, there is a statistically significant reduction of the RSV RNA copy number in the antibody-treated groups as compared to the Placebo-treated control (p<0.05; homoscedastic t-test). In the second study, the virus load in the groups treated with the antibodies of the invention are significantly lower than in the Synagis-treated groups at the corresponding doses (Table 7b). In the third study, the virus load is significantly lower in the groups treated with the anti-RSV rpAb 33 than in the Synagis-treated groups at all tested doses (Table 7c).

TABLE 7b
Virus loads in the lungs of mice following
prophylaxis and RSV challenge.

| | Virus load by RT-PCR |
|---|---|
| Treatment group (dose) | (log10 RSV RNA copies/ng total RNA) |
| Uninfected | Negative |
| PBS | 4.22 ± 0.20 |
| Synagis (15 mg/kg) | 3.68 ± 0.25 |
| Synagis (3 mg/kg) | 3.83 ± 0.12 |
| Anti-RSV rpAb 28 (15 mg/kg) | 2.96 ± 0.19 |
| Anti-RSV rpAb 28 (3 mg/kg) | 3.32 ± 0.23 |
| Anti-RSV rpAb 33 (15 mg/kg) | 2.95 ± 0.30 |
| Anti-RSV rpAb 33 (3 mg/kg) | 3.66 ± 0.07 |
| Anti-RSV rpAb 56 (15 mg/kg) | 2.66 ± 0.18 |
| Anti-RSV rpAb 56 (3 mg/kg) | 3.25 ± 0.38 |
| Clone 824 (15 mg/kg) | 2.51 ± 0.28 |
| Clone 824 (3 mg/kg) | 3.09 ± 0.18 |

TABLE 7c
Virus loads in the lungs of mice following
prophylaxis and RSV challenge.

| | Virus load by RT-PCR |
|---|---|
| Treatment group (dose) | (log10 RSV RNA copies/ng total RNA) |
| Uninfected | Negative |
| PBS | 4.13 ± 0.17 |
| Synagis (45 mg/kg) | 3.56 ± 0.22 |
| Synagis (15 mg/kg) | 3.60 ± 0.27 |
| Synagis (5 mg/kg) | 3.77 ± 0.14 |
| Synagis (1.5 mg/kg) | 3.86 ± 0.12 |
| Anti-RSV rpAb 33 (45 mg/kg) | 2.38 ± 0.18 |
| Anti-RSV rpAb 33 (15 mg/kg)* | 2.70 ± 0.18 |
| Anti-RSV rpAb 33 (5 mg/kg)* | 3.15 ± 0.24 |
| Anti-RSV rpAb 33 (1.5 mg/kg)* | 3.53 ± 0.12 |

The asterisk indicates that the group only contained four animals.

### Example 4

### Derivation of cho Cell

### Derivation of CHO Cell Clones Expressing Antibodies

### Expression Vector

The IgG expression vector used is shown in FIG. 2a.

The E1A expression vector is shown in FIG. 2b.

### Cell Line

The cell line used is a derivative of the DHFR-negative CHO cell line DG44 obtained from Lawrence Chasin, Columbia University (also available from Gibco cat # 12613-014). DG44 cells were transfected with a cDNA for the 13S version of the adenovirus type 5 transactivator E1A (NO31 accession no. AY339865, cDNA sequence: atgagacatattatctgccacggaggtgttattaccgaagaaatggccgccagtatttggaccagctgatcgaagaggtactggc tgataatcttccacctcctagccattttgaaccacctacccttcacgaactgtatgatttagacgtgacggcccccgaagatcccaa cgaggaggcggatcgcagatttttcccgactctgtaatgttggcggtgcaggaagggattgacttactcacttttccgccggcgc ccggttctccggagccgcctcacctttcccggcagcccgagcagccggagcagagagccttgggtccggtttctatgccaaac cttgtaccggaggtgatcgatcttacctgccacgaggctggctttccacccagtgacgacgaggatgaagagggtgaggagttt gtgttagattatgtggagcaccccgggcacggttgcaggtatgtcattatcaccggaggaatacgggggacccagatattatgtg ttcgctttgctatatgaggacctgtggcatgtttgtctacagtcctgtgtctgaacctgagcctgagcccgagccagaaccggagc ctgcaagacctacccgccgtcctaaaatggcgcctgctatcctgagacgcccgacatcacctgtgtctagagaatgcaatagtag tacggatagctgtgactccggtccttctaacacacctcctgagatacacccggtggtcccgctgtgccccattaaaccagttgccg tgagagttggtgggcgtcgccaggctgtggaatgtatcgaggacttgcttaacgagcctgggcaacctttggacttgagctgtaa acgccccaggccataa) in the vector pcDNA3.1+(Cat # V790-20, Invitrogen). Transfectants were selected with Geneticin (Invitrogen) at a concentration of 500 μg/ml. After selection the cells were single-cell cloned by limiting dilution. Clones were tested for antibody expression by transient transfection with an antibody plasmid (shown above). A single clone showed an expression level in the transient assay that was improved by a factor of 3 compared to the untransfected DG44 cell line. In comparisons performed with stable transfection, selected pools showed a 4-5 times increased expression level compared to the wild-type DG44 cell line. This clone (termed ECHO) was subcloned twice and appeared to be stable with regard to high expression of antibody.

### Example 5

### Establishment of Anti-RSV Antibody Expressing Cell Lines with Randomly Integrated Expression Vectors

Antibody Expression Plasmids

13 different anti-RSV antibodies were chosen for expression in the ECHO cell line. The antibody expression plasmids used were constructed as shown above. The antibodies were:
Sym003-810 (clone 810)
Sym003-818 (clone 818)
Sym003-819 (clone 819)
Sym003-824 (clone 824)
Sym003-825 (clone 825)
Sym003-827 (clone 827)
Sym003-858 (clone 858)
Sym003-894 (clone 894)
Sym003-793 (clone 793)
Sym003-816 (clone 816)
Sym003-853 (clone 853)
Sym003-855 (clone 855)
Sym003-856 (clone 856)

The clone numbers refer to the numbers in Table 3. The light chain and V_{H} polypeptide and encoding sequences for the clones are found in Example 1. The C_{H} sequence is found in SEQ ID NO 177, and its coding sequence in SEQ ID NO 178. The general procedure for transfection of ECHO cells with anti-RSV antibody expressing plasmids is illustrated below.

### IgG ELISA

IgG was measured by sandwich ELISA. Briefly, 96-well plates (Maxisorp,

NUNC) were coated with goat anti-human Fe (Serotec, STAR106) followed by incubation with samples and standard (purified human monoclonal IgG1 kappa antibody). Detection was performed with goat anti-human kappa light chains conjugated with horseradish peroxidase (Serotec STAR100P).

### Transfection of ECHO Cells

ECHO cells were seeded in T75 flasks at a density of 0.15*10⁶ cells/per flask in MEM alpha medium with nucleosides (Invitrogen cat.no. 32571) with 10% fetal calf serum (FCS) (Invitrogen). On the following day, the cells were transfected with Fugene6 (Roche):

10 μl of Fugene6 is mixed with 490 μl Dulbecco's modified Eagle's medium and allowed to incubate for 5 min at room temperature

5 μg of expression plasmid is added and the mix is incubated for a further 15 min at room temperature

The mix is added to the cell culture flask

24 hrs after transfection the medium with transfection reagents was aspirated, each flask was washed once with 5 ml of MEM alpha medium (without nucleosides; MEMalpha-) with 10% dialyzed FCS (Invitrogen). 10 ml of the same medium (MEMalpha- with 10% dialysed FCS) was added together with methotrexate at a concentration of 3 nM for selection. Following this the medium was changed three times a week.

Around day 14 to 18 the cells were trypsinized, resuspended in 10 ml selective medium and transferred to a new T75 or T175 flask.

The next day the medium was changed and after 24 h a medium sample was aspirated for ELISA and the cells were trypsinized, counted and transferred to a new T-flasks in MEMalpha- with 3nM methotrexate. Productivity was measured by performing IgG ELISA on supernatants. Before the cells reached confluency the pools of cells were frozen in culture medium containing 20% DMSO and 10% dialyzed FCS.

For the production of single-cell clones the pools were thawed again. Cells may also be subjected to single-cell cloning without a prior freezing step. After 3 days cells were stained for surface-associated antibody and single-cell sorted into 96-well plates containing 50% ECHO-cell conditioned medium (MEMalpha-) and 50% of the same medium without conditioning. Briefly, the staining protocol was as follows:
1. Cells were trypsinized and counted
2. 1-5×10⁶ cells were pipetted into sterile FACS tube
3. cells were spun down for 1 min at 250 g 4° C. and remove supernatant
4. cells were washed in 2 ml sterile FACS PBS (PBS+2% FCS) (5 ml)
5. cells were stained with (Goat F(ab)2 fragment anti-human IgG H+L− PE (Beckman-Coulter, IM1626) diluted 1:20 in 100 μl diluted Ab/106 cells and incubated for 20 min (4° C. in the dark)
6. cells were washed twice in 2 ml FACS PBS (5 ml)
7. cells were resuspended to 1-5×10⁶/ml in FACS PBS (2 ml)
8. propidium iodide was added, 10 μg/ml 1:100

30,000 events were recorded and high expressing cells were identified for Sym003-824, using the following gating strategy: Firstly, a gate (p1) was set in the fsc/ssc dot plot gating cells of approximately same size and granularity. Then, live cells were gated (p2) using propidium iodide staining as a marker of dead cells. Thirdly, multimeric cell clumps are excluded using the doublet discrimination technique with ssc-hight and ssc-width (p3). Finally, a gate (P5) was set including the 0.2% strongest stained cells.

Using this gating, cells were single-cell sorted into 96-well plates (5 plates per antibody) using a FACS-Aria (Beckton-Dickinson).

After 7 days wells were inspected by microscope for the presence of single clones. After inspection 100 μl MEMalpha medium with 10% dialysed FCS was added to each well.

12 days after sorting supernatants from wells with a single clone were assayed each in a single dilution by IgG ELISA. Based on the IgG ELISA value and visual inspection (cell number and morphology) of the wells 15-25 clones representing each antibody were selected for continued culture. Selected clones were trypsinized and transferred to 6-well plate and further to T75 flask when the 6-wells were close to confluency. When the cultures were ≧50% confluent, the medium was changed. 24 hours later IgG ELISA was performed on the supernatant and the cells were counted. A number of clones with an appropriate productivity was chosen for freezing and adaptation to suspension culture.

### Adaptation to Serum-Free Suspension Culture

Cells were trypsinized and counted. 6*10⁶ cells were centrifuged and resuspended in 12 ml ProCHO4 serum-free medium (Lonza). The cells were transferred to 50 ml cell culture tubes (TRP, Switzerland) and incubated on a shaker at 37° C. Cell densities were counted twice a week for at least 2 weeks and each time the cultures were diluted to 0.5*10⁶ cells per ml if possible. When doubling times were stably below 60 h the cells were diluted 3 times a week for 0.5*10⁶ cells per ml.

Specific productivity (pictogram/cell/24 h) was determined by IgG ELISA on a supernatant sample which was taken after dilution of the culture and a supernatant sample was taken 48 hours afterwards. High-expression clones continued in adaptation.

After 6 to 8 weeks doubling times for most clones were approaching 35 h at which time point it was considered that they were adapted to serum-free culture. From then the cells were cultivated in shaker flasks by diluting the cultures 3 times a week to 0.5*10⁶ cells per ml. The culture volume was stepwise scaled up to 150 ml.

Suspension cells were frozen in freezing medium (50% conditioned medium: 50% fresh culture medium+7.5% DMSO). To ensure that the cells were in exponential growth before freezing the doubling time during the last 24 hours before freezing had to be 35 h or less. Specific productivity on the day of freezing was determined by IgG ELISA as described above.

Adapted cell clones were prepared for each of the 13 anti-RSV antibodies.

### Example 6

### Purification and Preliminary Characterization of Individual Sym003 Antibodies Expressed in the ECHO Cell Line

The recombinant antibody samples were purified by affinity chromatography using MAb Select SuRe (GE Healthcare, UK). The culture supernatants from shaker flasks, pre-clarified by centrifugation and filtration using 0.22 μm filters, were purified using 0.1 ml MAb Select SuRe packed in small single use columns. Briefly, the MAb Select SuRe column was equilibrated in PBS buffer, pH 7.4. The culture supernatant was applied onto the column at RT using gravity flow rate. The column was subsequently washed using PBS, pH 7.4 using gravity flow rate and eluted using 0.1 M Glycine-HCl, pH 2.7 also using gravity flow rate. The purified antibody samples were neutralized by addition of 1 M Tris, pH 7.0 and further analysed using SDS-PAGE. The purified amounts were typically between 10 to 250 μg.

FIG. 3 shows an example of SDS-PAGE of antibodies 818, 810, and two clones of 824 (824-8 and 824-17).

### Example 7

### Establishment of a Polyclonal Cell Bank

To establish a polyclonal cell bank capable of expressing several antibodies in the same vessel, mixes of clones are prepared. Based on cell counts made during the adaptation period doubling time is taken into consideration to the extent possible. Care is taken to match clones with similar doubling time.

Clones are mixed so that the number of cells representing each antibody constitute the same percentage of the total number of cells in the mix.

1-38. (canceled)

39. A polyclonal cell line comprising two or more sub-populations of cells, wherein each sub-population of cells expresses a distinct antibody member of a recombinant polyclonal anti-RSV antibody and comprises at least one expression construct encoding the distinct antibody member randomly and stably integrated into the genome, wherein the distinct antibody members of the polyclonal cell line comprise:
(i) a heavy chain CDR1 comprising an amino acid sequence selected from SEQ ID NOs:201-285; a heavy chain CDR2 comprising an amino acid sequence selected from SEQ ID NOs:286-370; and/or a heavy chain CDR3 comprising an amino acid sequence selected from SEQ ID NOs:371-455; and/or
(ii) a light chain CDR1 comprising an amino acid sequence selected from SEQ ID NOs:456-540; a light chain CDR2 comprising an amino acid sequence selected from SEQ ID NOs:541-625; and/or a light chain CDR3 comprising an amino acid sequence selected from SEQ ID NOs:626-710.

40. The polyclonal cell line of claim 39, wherein the distinct antibody members comprise the antibodies encoded by:
(a) clones 810, 818, 819, 825, and 827;
(b) clones 810, 818, 819, 825, 827, 831, 858, 863, 884, 894, 793, 796, 816, 838, 853, 856, 859, and 888;
(c) clones 810, 818, 825, 827, 884, 886, 793, 853, 868, and 888;
(d) clones 810, 818, 825, 827, 831, 858, 884, 886, 793, 796, 816, 853, 856, 868, 888;
(e) clones 810, 818, 825, 827, 831, 858, 884, 886, 793, 853, 868, and 888;
(f) clones 810, 819, 825, 827, 831, 793, 853, 856, 858, and 868;
(g) clones 810, 811, 817, 819, 825, 827, 831, 838, 853, 856, 858, 859, 863, and 868;
(h) clones 800, 801, 811, 838, 853, 855, 859, 861, 880, 894, 736, 795, 796, and 799;
(i) clones 810, 818, and 825;
(j) clones 810, 818, 819, 825, 827, and 884;
(k) clones 810, 818, 819, 825, 827, 884, and 886;
(l) clones 793, 816, 853, and 856;
(m) clones 793, 816, 853, 855, and 856;
(n) clones 793, 868, 888, 853, and 856;
(o) clones 793, 796, 818, 816, 838, 853, 855, 856, 859, 868, and 888;
(p) clones 810, 818, and 827;
(q) clones 810, 818, 825, 827, 858, and 886;
(r) clones 810, 818, 825, 827, 858, 886, 793, 816, 853, 855, and 856;
(s) clones 818, 825, 827, 858, 886, 793, 816, 853, 855, and 856;
(t) clones 810, 818, 819, 825, 827, 858, 793, 816, 853, 855, and 856;
(u) clones 810, 793, 816, 853, 855, and 856;
(v) clones 818, 825, 827, 831, 858, 886, 793, 816, 853, 855, and 856;
(w) clones 818, 825, 827, 831, 858, 819, 793, 816, 853, 855, and 856;
(x) clones 818, 827, 831, 858, 819, 793, 816, 853, 855, and 856;
(y) clones 810, 818, 819, 824, 825, 827, 858, 793, 816, 853, 855, and 856;
(z) clones 831, 818, 819, 824, 825, 827, 858, 793, 816, 853, 855, and 856;
(aa) clones 831, 818, 819, 824, 827, 858, 793, 816, 853, 855, and 856;
(bb) clones 810, 818, and 824;
(cc) clones 810 and 824;
(dd) clones 818 and 824;
(ee) clones 810 and 818;
(ff) clones 824, 793, 816, 853, 855, and 856;
(gg) clones 810, 818, 819, 824, 825, 827, 858, 894, 793, 816, 853, 855, and 856;
(hh) clones 810, 818, 819, 824, 825, 827, 894, 793, 816, 853, 855, and 856;
(ii) clones 793 and 816;
(jj) clones 855 and 856;
(kk) clones 793 and 856;
(ll) clones 793 and 853;
(mm) clones 853 and 856;
(nn) clones 793, 853, and 856;
(oo) clones 793, 816, and 853;
(pp) clones 853, 855, and 856;
(qq) clones 793, 816, 853, and 856;
(rr) clones 793, 853, and 855;
(ss) clones 793, 853, 855, and 856;
(tt) clones 816, 853, 855, and 856;
(uu) clones 816 and 856;
(vv) clones 816 and 853;
(ww) clones 816, 853, and 856;
(xx) clones 810, 818, 824, and 816;
(yy) clones 810, 818, 824, and 853;
(zz) clones 810, 818, 824, and 856;
(aaa) clones 810, 818, 824, 816, and 853;
(bbb) clones 810, 818, 824, 816, and 856;
(ccc) clones 810, 818, 824, 853, and 856; or
(ddd) clones 810, 818, 824, 816, 853, and 856.

41. The polyclonal cell line of claim 40, wherein the distinct antibody members comprise the antibodies encoded by:
(a) clones 810, 818 and 824;
(b) clones 810, 818, 819, 824, 825, 827, 858, 894, 793, 816, 853, 855, and 856; or
(c) clones 810, 818, 824, 816, 853, and 856.

42. The polyclonal cell line of claim 39, wherein the distinct antibody members comprise antibodies comprising the V_{H} and V_{L} sequences encoded by clones 735, 736, 744, 793, 795, 796, 799, 800, 801, 804, 810, 811, 812, 814, 816, 817, 818, 819, 824, 825, 827, 829, 830, 831, 835, 838, 841, 853, 855, 856, 857, 858, 859, 861, 863, 868, 870, 871, 880, 881, 884, 886, 888, or 894.

43. The polyclonal cell line of claim 42, wherein the distinct antibody members comprise the antibodies encoded by clones 793, 800, 810, 816, 818, 819, 824, 825, 827, 831, 853, 855, 856, 858, 868, 880, 888, or 894, or antibodies comprising the CDRs of said antibodies.

44. The polyclonal cell line of claim 43, wherein the distinct antibody members comprise antibodies comprising the V_{H} and V_{L} sequences encoded by clones 810, 818, 819, 824, 825, 827, 858, 894, 793, 816, 853, 855, or 856.

45. The polyclonal cell line of claim 39, wherein at least one distinct antibody member is the antibody encoded by clone 824 or an antibody comprising the CDRs of the antibody encoded by clone 824.

46. The polyclonal cell line of claim 39, wherein at least one distinct antibody member is the antibody encoded by clone 810 or an antibody comprising the CDRs of the antibody encoded by clone 810.

47. The polyclonal cell line of claim 39, wherein at least one distinct antibody member binds to F protein, and at least one distinct antibody member binds to G-protein.

48. The polyclonal cell line of claim 39, comprising three or more sub-populations of cells.

49. The polyclonal cell line of claim 39, comprising less than 30 sub-populations of cells.

50. The polyclonal cell line of claim 39, wherein the sub-population of cells are derived from one or more cloned cells.

51. The polyclonal cell line of claim 39, wherein the expression construct encodes both the heavy and light chains of the distinct antibody member.

52. The polyclonal cell line of claim 39, wherein separate expression constructs encode the heavy and light chains of the distinct antibody member.

53. The polyclonal cell line of claim 51, wherein expression of the heavy and light chains of the distinct antibody member is under the control of the same promoter.

54. The polyclonal cell line of claim 51, wherein the expression construct encodes a selectable marker.

55. The polyclonal cell line of claim 54, wherein the selectable marker is encoded by a transcript that also encodes an antibody or an antibody subunit.

56. The polyclonal cell line of claim 39, wherein the distinct antibody members are of the same isotype.

57. The polyclonal cell line of claim 39, wherein the sub-populations of cells are prokaryotic.

58. The polyclonal cell line of claim 39, wherein the sub-populations of cells are eukaryotic.

59. A cell comprising an expression construct that directs the expression of an anti-RSV antibody selected from an antibody comprising:
(i) a heavy chain CDR1 comprising an amino acid sequence selected from SEQ ID NOs:201-285; a heavy chain CDR2 comprising an amino acid sequence selected from SEQ ID NOs:286-370; and/or a heavy chain CDR3 comprising an amino acid sequence selected from SEQ ID NOs:371-455; and/or
(ii) a light chain CDR1 comprising an amino acid sequence selected from SEQ ID NOs:456-540; a light chain CDR2 comprising an amino acid sequence selected from SEQ ID NOs:541-625; and/or a light chain CDR3 comprising an amino acid sequence selected from SEQ ID NOs:626-710;
wherein the cell comprises at least one expression construct stably integrated at a random position in the genome.

60. The cell of claim 59, wherein the anti-RSV antibody is selected from antibodies comprising the CDRs from the V_{H} and V_{L} sequences encoded by clones 735, 736, 744, 793, 795, 796, 799, 800, 801, 804, 810, 811, 812, 814, 816, 817, 818, 819, 824, 825, 827, 829, 830, 831, 835, 838, 841, 853, 855, 856, 857, 858, 859, 861, 863, 868, 870, 871, 880, 881, 884, 886, 888, or 894.

61. The cell of claim 59, wherein the cell comprises two or more expression constructs integrated at different and random positions into the genome.

62. The cell of claim 59, wherein the anti-RSV antibody is selected from the antibodies encoded by clones 793, 800, 810, 816, 818, 819, 824, 825, 827, 831, 853, 855, 856, 858, 868, 880, 888, or 894, or antibodies comprising the CDRs of said antibodies.

63. The cell of claim 59, wherein the anti-RSV antibody is selected from the antibodies encoded by clones 793, 800, 810, 818, 819, 824, 825, 827, 831, 853, 858, 888, or 894.

64. The cell of claim 59, wherein the anti-RSV antibody comprises the CDR sequences encoded by clone 810.

65. The cell of claim 59, wherein the anti-RSV antibody comprises the CDR sequences encoded by clone 824.

66. The cell of claim 59, wherein the cell is eukaryotic.

67. The cell of claim 66, wherein the eukaryotic cell is from a plant, yeast, fungus, vertebrate, or invertebrate.

68. The cell of claim 66, wherein the eukaryotic cell is selected from Chinese hamster ovary (CHO) cells, COS cells, BHK cells, myeloma cells, NIH 3T3 cells, HeLa cells, HEK293 cells, and PER.C6 cells.

69. A method for generating a cell that expresses an anti-RSV antibody, comprising:
transfecting the cell with an expression construct encoding the anti-RSV antibody under conditions allowing random integration into the genome of the cell; and
selecting at least one transfected cell having the expression construct integrated stably at a random position;
wherein the anti-RSV antibody is an antibody comprising:
(i) a heavy chain CDR1 comprising an amino acid sequence selected from SEQ ID NOs:201-285; a heavy chain CDR2 comprising an amino acid sequence selected from SEQ ID NOs:286-370; and/or a heavy chain CDR3 comprising an amino acid sequence selected from SEQ ID NOs:371-455; and/or
(ii) a light chain CDR1 comprising an amino acid sequence selected from SEQ ID NOs:456-540; a light chain CDR2 comprising an amino acid sequence selected from SEQ ID NOs:541-625; and a light chain CDR3 comprising an amino acid sequence selected from SEQ ID NOs626-710.

70. The method of claim 69, wherein the transfection and/or selection is carried out under conditions favoring amplification of the expression construct.

71. A method for manufacture of a polyclonal anti-RSV antibody, comprising:
providing the polyclonal cell line of claim 39;
culturing the polyclonal cell line under conditions allowing for expression of the distinct antibody members; and
recovering and optionally purifying the polyclonal anti-RSV antibody from the culture medium.

72. The method of claim 71, wherein the culturing is in a shake flask, a disposable bioreactor, or a bioreactor.

73. The method of claim 71, comprising at least two polyclonal cell lines, wherein a first polyclonal cell line is cultured in a first container, and a second polyclonal cell line is cultured in a second container, and wherein the first polyclonal cell line and the second polyclonal cell line are mixed together prior to or after purification.

74. The method of claim 71, further comprising verifying the presence of the polyclonal anti-RSV antibody.

75. A method for manufacture of an anti-RSV antibody, comprising:
providing a cell line derived from the cell of claim 59;
culturing the cell line under conditions allowing for expression of the anti-RSV antibody; and
recovering and optionally purifying the anti-RSV antibody from the culture medium.

76. The method of claim 75, wherein the culturing is a shake flask, a disposable bioreactor, or a bioreactor.


SEQUENCE LISTING (714 sequences)

SEQ ID NO: 1 - protein, 122 aa, homo sapiens
QVQLQESGPGLVKPSETLSLTCTVSNGAIGDYDWSWIRQSPGKGLEWIGNINYRGNTNYNPSLKSRVTMSLRTSTMQFSLKLSSATAADTAVYYCARDVGYGGGQYFAMDVWSPGTTVTVSS

SEQ ID NO: 2 - protein, 129 aa, homo sapiens
QVQLVESGGGVVQPGGSLRLSCTASGFTFSTYGMHWVRQAPGKGLEWVAFIRYDGSTQDYVDSVKGRFTISRDNSKNMVYVQMNSLRVEDTAVYYCAKDMDYYGSRSYSVTYYYGMDVWGQGTTVTVSS

SEQ ID NO: 3 - protein, 125 aa, homo sapiens
QVQLVQSGAEVKKPGASVKVSCKASGYTFSGYYMHWVRQAPGQGLEWMGWINTSSGGTNYAQKFQGRVTMTRDTSISTAHMELRRLRSDDTAVYYCAREDGTMGTNSWYGWFDPWGQGTLVTVSS

SEQ ID NO: 4 - protein, 127 aa, homo sapiens
QVQLVESGGGLVKPGGSLRLSCAASGFPFGDYYMSWIRQAPGKGLEWVAYINRGGTTIYYADSVKGRFTISRDNAKNSLFLQMNSLRAGDTALYYCARGLILALPTATVELGAFDIWGQGTMVTVSS

SEQ ID NO: 5 - protein, 126 aa, homo sapiens
QVQLQESGPGLVKPSQTLSLTCTVSGASISSGDYYWSWIRQSPRKGLEWIGYIFHSGTTYYNPSLKSRAVISLDTSKNQFSLRLTSVTAADTAVYYCARDVDDFPVWGMNRYLALWGRGTLVTVSS

SEQ ID NO: 6 - protein, 124 aa, homo sapiens
QVQLVESGGGVVQPGRSLRLSCAASGFSFSHFGMHWVRQVPGKGLEWVAIISYDGNNVHYADSVKGRFTISRDNSKNTLFLQMNSLRDDDTGVYYCAKDDVATDLAAYYYFDVWGRGTLVTVSS

SEQ ID NO: 7 - protein, 123 aa, homo sapiens
QVQLVESGGGVVQPGRSLKLSCEASGFNFNNYGMHWVRQAPGKGLEWVAVISYDGRNKYFADSVKGRFIISRDDSRNTVFLQMNSLRVEDTAVYYCARGSVQVWLHLGLFDNWGQGTLVTVSS

SEQ ID NO: 8 - protein, 122 aa, homo sapiens
QVQLVESGGAVVQPGRSLRLSCEVSGFSFSDYGMNWVRQGPGKGLEWVAVIWHDGSNKNYLDSVKGRFTVSRDNSKNTLFLQMNSLRAEDTAVYYCARTPYEFWSGYYFDFWGQGTLVTVSS

SEQ ID NO: 9 - protein, 116 aa, homo sapiens
QVQLVESGGGVVQPGRSLRLSCAASGFPFNSYAMHWVRQAPGKGLEWVAVIYYEGSNEYYADSVKGRFTISRDNSKNTLYLQMDSLRAEDTAVYYCARKWLGMDFWGQGTLVTVSS

SEQ ID NO: 10 - protein, 120 aa, homo sapiens
EVQLVESGGGLVRPGGSLRLSCSASGFTFSNYAMHWVRQAPGKRLEYVSATSTDGGSTYYADSLKGTFTISRDNSKNTLYLQMSSLSTEDTAIYYCARRFWGFGNFFDYWGRGTLVTVSS

SEQ ID NO: 11 - protein, 122 aa, homo sapiens
QVQLVQSGAEVKKSGSSVKVSCRASGGTFGNYAINWVRQAPGQGLEWVGRIIPVFDTTNYAQKFQGRVTITADRSTNTAIMQLSSLRPQDTAMYYCLRGSTRGWDTDGFDIWGQGTMVTVSS

SEQ ID NO: 12 - protein, 129 aa, homo sapiens
QVQLVQSGAVVETPGASVKVSCKASGYIFGNYYIHWVRQAPGQGLEWMAVINPNGGSTTSAQKFQDRITVTRDTSTTTVYLEVDNLRSEDTATYYCARQRSVTGGFDAWLLIPDASNTWGQGTMVTVSS

SEQ ID NO: 13 - protein, 126 aa, homo sapiens
QVQLVQSGAEMKKPGSSVKVSCKASGGSFSSYSISWVRQAPGRGLEWVGMILPISGTTNYAQTFQGRVIISADTSTSTAYMELTSLTSEDTAVYFCARVFREFSTSTLDPYYFDYWGQGTLVTVSS

SEQ ID NO: 14 - protein, 125 aa, homo sapiens
QVQLVESGGGVVQPGKSVRLSCVGSGFRLMDYAMHWVRQAPGKGLDWVAVISYDGANEYYAESVKGRFTVSRDNSDNTLYLQMKSLRAEDTAVYFCARAGRSSMNEEVIMYFDNWGLGTLVTVSS

SEQ ID NO: 15 - protein, 127 aa, homo sapiens
EVQLLESGGGLVQPGGSLRLSCVASGFTFSTYAMTWVRQAPGKGLEWVSVIRASGDSEIYADSVRGRFTISRDNSKNTVFLQMDSLRVEDTAVYFCANIGQRRYCSGDHCYGHFDYWGQGTLVTVSS

SEQ ID NO: 16 - protein, 127 aa, homo sapiens
QVQLVESGGGVVQPGRSLRLSCAASGFGFNTHGMHWVRQAPGKGLEWLSIISLDGIKTHYADSVKGRFTISRDNSKNTVFLQLSGLRPEDTAVYYCAKDHIGGTNAYFEWTVPFDGWGQGTLVTVSS

SEQ ID NO: 17 - protein, 126 aa, homo sapiens
QVTLRESGPAVVKPTETLTLTCAFSGFSLNAGRVGVSWIRQPPGQAPEWLARIDWDDDKAFRTSLKTRLSISKDSSKNQVVLTLSNMDPADTATYYCARTQVFASGGYYLYYLDHWGQGTLVTVSS

SEQ ID NO: 18 - protein, 129 aa, homo sapiens
QVQLQESGPGLVKPSQTLSLTCTVSSGAISGADYYWSWIRQPPGKGLEWVGFIYDSGSTYYNPSLRSRVTISIDTSKKQFSLKLTSVTAADTAVYYCARDLGYGGNSYSHSYYYGLDVWGRGTTVTVSS

SEQ ID NO: 19 - protein, 119 aa, homo sapiens
QVQLQESGPGLVKPSETLSLTCTVSGGSIGNYYWGWIRQPPGKGLEWIGHIYFGGNTNYNPSLQSRVTISVDTSRNQFSLKLNSVTAADTAVYYCARDSSNWPAGYEDWGQGTLVTVSS

SEQ ID NO: 20 - protein, 123 aa, homo sapiens
QVQLVQSGAEVKKPGASVKVSCKVSGYTFTSNGLSWVRQAPGQGFEWLGWISASSGNKKYAPKFQGRVTLTTDISTSTAYMELRSLRSDDTAVYYCAKDGGTYVPYSDAFDFWGQGTMVTVSS

SEQ ID NO: 21 - protein, 118 aa, homo sapiens
QVQLVQSGAEVKKPGASVKVSCRVSGHTFTALSKHWMRQGPGGGLEWMGFFDPEDGDTGYAQKFQGRVTMTEDTATGTAYMELSSLTSDDTAVYYCATVAAAGNFDNWGQGTLVTVSS

SEQ ID NO: 22 - protein, 126 aa, homo sapiens
QVTLKESGPALVKATQTLTLTCTFSGFSLSRNRMSVSWIRQPPGKALEWLARIDWDDDKFYNTSLQTRLTISKDTSKNQVVLTMTNMDPVDTATYYCARTGIYDSSGYYLYYFDYWGQGTLVTVSS

SEQ ID NO: 23 - protein, 130 aa, homo sapiens
QVQLVQSGAEVKVPGASVKVSCKASGYTFTTYGVSWVRQAPGQGLEWMGWISAYNGNTYYLQKLQGRVTMTTDTSTSTAYMELRGLRSDDTAMYYCARDRVGGSSSEVLSRAKNYGLDVWGQGTTVTVSS

SEQ ID NO: 24 - protein, 123 aa, homo sapiens
QVQLVQSGAEVKKPGASVKVSCKASANIFTYAMHWVRQAPGQRLEWMGWINVGNGQTKYSQRFQGRVTITRDTSATTAYMELSTLRSEDTAVYYCARRASQYGEVYGNYFDYWGQGTLVTVSS

SEQ ID NO: 25 - protein, 126 aa, homo sapiens
QVQLVQSGAEVKRPGASVKVSCKASGYTFISYGFSWVRQAPGQGLEWMGWSSVYNGDTNYAQKFHGRVNMTTDTSTNTAYMELRGLRSDDTAVYFCARDRNVVLLPAAPFGGMDVWGQGTMVTVSS

SEQ ID NO: 26 - protein, 123 aa, homo sapiens
QVQLVESGGGVVQPGTSLRLSCAASGFTFSTFGMHWVRQAPGKGLEWVAVISYDGNKKYYADSVKGRFTISRDNSKNTLYLQVNSLRVEDTAVYYCAAQTPYFNESSGLVPDWGQGTLVTVSS

SEQ ID NO: 27 - protein, 126 aa, homo sapiens
QVQLVQSGAEVKKPGASVKVSCKASGYTFISFGISWVRQAPGQGLEWMGWISAYNGNTDYAQRLQDRVTMTRDTATSTAYLELRSLKSDDTAVYYCTRDESMLRGVTEGFGPIDYWGQGTLVTVSS

SEQ ID NO: 28 - protein, 128 aa, homo sapiens
EVQLVQSGAEVKKPGQSLKISCKTSGYIFTNYWIGWVRQRPGKGLEWMGVIFPADSDARYSPSFQGQVTISADKSIGTAYLQWSSLKASDTAIYYCARPKYYFDSSGQFSEMYYFDFWGQGTLVTVSS

SEQ ID NO: 29 - protein, 119 aa, homo sapiens
QVQLVQSGPEVKKPGASVKVSCKASGYVLTNYAFSWVRQAPGQGLEWLGWISGSNGNTYYAEKFQGRVTMTTDTSTSTAYMELRSLRSDDTAVYFCARDLLRSTYFDYWGQGTLVTVSS

SEQ ID NO: 30 - protein, 126 aa, homo sapiens
QVQLVQSGAEVKKPGASVKVSCKASGYTFSNYGFSWVRQAPGRGLEWMGWISAYNGNTYYAQNLQGRVTMTTDTSTTTAYMVLRSLRSDDTAMYYCARDGNTAGVDMWSRDGFDIWGQGTMVTVSS

SEQ ID NO: 31 - protein, 131 aa, homo sapiens
EVQLLESGGGLVQPGGPLRLSCVASGFSFSSYAMNWIRLAPGKGLEWVSGISGSGGSTYYGDSVKGRFTISRDNSKNTLYLQMNSLRAEDTAVYYCAKEPWIDIVVASVISPYYYDGMDVWGQGTTVTVSS

SEQ ID NO: 32 - protein, 123 aa, homo sapiens
QVQLVQSGAEVKKPGSSVKVSCKASGGSFDGYTISWLRQAPGQGLEWMGRVVPTLGFPNYAQKFQGRVTVTADRSTNTAYLELSRLTSEDTAVYYCARMNLGSHSGRPGFDMWGQGTLVTVSS

SEQ ID NO: 33 - protein, 129 aa, homo sapiens
QVQLVESGGGVVQPGRSLRLSCAVSGSSFSKYGIHWVRQAPGKGLEWVAVISYDGSKKYFTDSVKGRFTIARDNSQNTVFLQMNSLRAEDTAVYYCATGGGVNVTSWSDVEHSSSLGYWGLGTLVTVSS

SEQ ID NO: 34 - protein, 125 aa, homo sapiens
QVQLVESGGGVVQPGGSLRLSCAASGFTFSSYGMHWVRQAPGKGLEWVAFIWNDGSNKYYADSVKGRFTISRDNSKNTLYLQMNSLRAEDTAVYYCVKDEVYDSSGYYLYYFDSWGQGTLVTVSS

SEQ ID NO: 35 - protein, 127 aa, homo sapiens
EVQLLESGGGLVQPGGSLRLSCAASGFTFSSYTMSWVRQAPGKGLEWVSSISASTVLTYYADSVKGRFTISRDNSKNTLYLQMSSLRAEDTAVYYCAKDYDFWSGYPGGQYWFFDLWGRGTLVTVSS

SEQ ID NO: 36 - protein, 123 aa, homo sapiens
QVQLQESGPGLVTPSETLSVTCTVSNYSIDNAYYWGWIRQPPGKGLEWIGSIHHSGSAYYNSSLKSRATISIDTSKNQFSLNLRSVTAADTAVYYCARDTILTFGEPHWFDPWGQGTLVTVSS

SEQ ID NO: 37 - protein, 124 aa, homo sapiens
QVQLQESGPGLVKPSETLSLTCTVSGDSISNYYWSWIRQPPGKGLEWIGEISNTWSTNYNPSLKSRVTISLDMPKNQLSLKLSSVTAADTAVYYCARGLFYDSGGYYLFYFQHWGQGTLVTVSS

SEQ ID NO: 38 - protein, 125 aa, homo sapiens
QVQLVESGGGVVQPGRSLRVSCAASGFTFSNYGMHWVRQAPGKGLEWVAVIWYDDSNKQYGDSVKGRFTISRDNSKSTLYLQMDRLRVEDTAVYYCARASEYSISWRHRGVLDYWGQGTLVTVSS

SEQ ID NO: 39 - protein, 126 aa, homo sapiens
QITLKESGPTLVRPTQTLTLTCTFSGFSLSTSKLGVGWIRQPPGKALEWLALVDWDDDRRYRPSLKSRLTVTKDTSKNQVVLTMTNMDPVDTATYYCAHSAYYTSSGYYLQYFHHWGPGTLVTVSS

SEQ ID NO: 40 - protein, 125 aa, homo sapiens
EVQLVESGGGVVQPGGSLRLSCEVSGFTFNSYEMTWVRQAPGKGLEWVSHIGNSGSMIYYADSVKGRFTISRDNAKNSLYLQMNSLRVEDTAVYYCARSDYYDSSGYYLLYLDSWGHGTLVTVSS

SEQ ID NO: 41 - protein, 120 aa, homo sapiens
QVQLVQSGAEVRKPGASVKVSCKASGHTFINFAMHWVRQAPGQGLEWMGYINAVNGNTQYSQKFQGRVTFTRDTSANTAYMELSSLRSEDTAVYYCARNNGGSAIIFYYWGQGTLVTVSS

SEQ ID NO: 42 - protein, 122 aa, homo sapiens
QVQLVESGGGVVQPGRSLRLSCAASGFSFSSYGMHWVRQAPGKGLEWVAVISNDGSNKYYADSVKGRFTISRDNSKKTMYLQMNSLRAEDTAVYFCAKTTDQRLLVDWFDPWGQGTLVTVSS

SEQ ID NO: 43 - protein, 130 aa, homo sapiens
QLQLQESGPGLVKPSETLSLTCTASGGSINSSNFYWGWIRQPPGKGLEWIGSIFYSGTTYYNPSLKSRVTISVDTSKNQFSLKLSPVTAADTAVYHCARHGFRYCNNGVCSINLDAFDIWGQGTMVTVSS

SEQ ID NO: 44 - protein, 122 aa, homo sapiens
QVQLVESGGGVVQPGKSLRLSCAASGFRFSDYGMHWVRQAPSKGLEWVAVIWHDGSNIRYADSVRGRFSISRDNSKNTLYLQMNSMRADDTAFYYCARVPFQIWSGLYFDHWGQGTLVTVSS

SEQ ID NO: 45 - DNA, 366 bp, homo sapiens
caggtgcagctgcaggagtcgggcccaggactggtgaagccttcggagaccctgtccctcacgtgcactgtgtctaatggcgccatcggcgactacgactggagctggattcgtcagtccccagggaagggactggagtggattgggaacataaattacagagggaacaccaactacaacccctccctcaagagtcgagtcaccatgtccctacgcacgtccacgatgcagttctccctgaagctgagctctgcgaccgctgcggacacggccgtctattactgtgcgagagatgtaggctacggtggcgggcagtatttcgcgatggacgtctggagcccagggaccacggtcaccgtctcgagt

SEQ ID NO: 46 - DNA, 387 bp, homo sapiens
caggtgcagctggtggagtctgggggaggcgtggtccagcctggggggtccctgagactctcctgtacagcgtctggattcaccttcagtacctatggcatgcactgggtccgccaggctcccggcaaggggctggaatgggtggcatttatacggtatgatggaagtactcaagactatgtagactccgtgaagggccgattcaccatctccagagacaattccaagaatatggtgtatgtgcagatgaacagcctgagagttgaggacacggctgtctattactgtgcgaaagacatggattactatggttcgcggagttattctgtcacctactactacggaatggacgtctggggccaagggaccacggtcaccgtctcgagt

SEQ ID NO: 47 - DNA, 375 bp, homo sapiens
caggtgcagctggtgcagtctggggctgaggtgaagaagcctggggcctcagtgaaggtctcctgcaaggcttctggatacaccttcagcggctattatatgcactgggtgcgacaggcccctggacaagggcttgagtggatgggatggatcaacactagcagtggtggcacaaactatgcgcagaagtttcagggcagggtcaccatgaccagggacacgtccatcagcacagcccacatggaactgaggaggctgagatctgacgacacggccgtgtattattgtgcgagagaggacggcaccatgggtactaatagttggtatggctggttcgacccctggggccagggaaccctggtcaccgtctcgagt

SEQ ID NO: 48 - DNA, 381 bp, homo sapiens
caggtgcagctggtggagtctgggggaggcttggtcaagcctggggggtccctgagactctcctgtgcggcctctggattccccttcggtgactactacatgagctggatccgccaggctccagggaagggactggagtgggttgcatacattaatagaggtggcactaccatatactacgcagactctgtgaagggccgattcaccatctccagggacaacgccaagaactccctgtttctgcaaatgaacagcctgagagccggggacacggccctctattactgtgcgagagggctaattctagcactaccgactgctacggttgagttaggagcttttgatatctggggccaagggacaatggtcaccgtctcgagt

SEQ ID NO: 49 - DNA, 378 bp, homo sapiens
caggtgcagctgcaggagtcgggcccaggactggtgaagccttcacagaccctgtccctcacctgcactgtctctggtgcctccatcagcagtggtgattattactggagttggatccgtcagtctccaaggaagggcctggagtggattgggtacatcttccacagtgggaccacgtactacaacccgtccctcaagagtcgagctgtcatctcactggacacgtccaagaaccaattctccctgaggctgacgtctgtgactgccgcagacacggccgtctattattgtgccagagatgtcgacgattttcccgtttggggtatgaatcgatatcttgccctctggggccggggaaccctggtcaccgtctcgagt

SEQ ID NO: 50 - DNA, 372 bp, homo sapiens
caggtgcagctggtggagtctgggggaggcgtggtccagcctgggaggtccctgagactctcctgtgcagcctctggattcagcttcagtcactttggcatgcactgggtccgccaggttccaggcaaggggctggagtgggtggcaattatatcatatgatgggaataatgtacactatgccgactccgtaaagggccgattcaccatctccagagacaattccaagaacacgctgtttctgcaaatgaacagcctgagagatgacgacacgggtgtgtattactgtgcgaaggacgacgtggcgacagatttggctgcctactactacttcgatgtctggggccgtggcaccctggtcaccgtctcgagt

SEQ ID NO: 51 - DNA, 369 bp, homo sapiens
caggtgcagctggtggagtctgggggcggcgtggtccagcctgggaggtccctgaaactctcttgtgaagcctctggattcaacttcaataattatggcatgcactgggtccgccaggcaccaggcaaggggctggagtgggtggcagttatttcatatgacggaagaaataagtattttgctgactccgtgaagggccgattcatcatctccagagacgattccaggaacacagtgtttctgcaaatgaacagcctgcgagttgaagatacggccgtctattactgtgcgagaggcagcgtacaagtctggctacatttgggactttttgacaactggggccagggaaccctggtcaccgtctcgagt

SEQ ID NO: 52 - DNA, 366 bp, homo sapiens
caggtgcagctggtggagtctgggggagccgtggtccagcctgggaggtccctgagactctcctgtgaagtgtctggattcagtttcagtgactatggcatgaactgggtccgccagggtccaggcaaggggctggagtgggtggcagttatatggcatgacggaagtaataaaaattatctagactccgtgaagggccgattcaccgtctccagagacaattccaagaacacattgtttctgcaaatgaacagcctgagagccgaagacacggctgtatattactgtgcgaggacgccttacgagttttggagtggctattactttgacttctggggccagggaaccctggtcaccgtctcgagt

SEQ ID NO: 53 - DNA, 348 bp, homo sapiens
caggtgcagctggtggagtctgggggaggcgtggtccagcctgggaggtccctgagactctcctgtgcagcgtctggattccccttcaatagctatgccatgcactgggtccgccaggctccaggcaaggggctggagtgggtggcagtgatatattatgaagggagtaatgaatattatgcagactccgtgaagggccgattcaccatctccagagacaattccaagaacactctgtatttgcaaatggatagcctgagagccgaggacacggctgtctattactgtgcgaggaagtggctggggatggacttctggggccagggaaccctggtcaccgtctcgagt

SEQ ID NO: 54 - DNA, 360 bp, homo sapiens
gaggtgcagctggtggagtctgggggaggcttggtccggcctggggggtccctgagactctcctgttcagcctctggattcaccttcagtaactatgctatgcactgggtccgccaggctccagggaagagactggaatatgtttcagctactagtactgatggggggagcacatactacgcagactccctaaagggcacattcaccatctccagagacaattccaagaacacactgtatcttcaaatgagcagtctcagtactgaggacacggctatttattactgcgcccgccgattctggggatttggaaacttttttgactactggggccggggaaccctggtcaccgtctcgagt

SEQ ID NO: 55 - DNA, 366 bp, homo sapiens
caggtgcagctggtgcagtctggggctgaggtgaagaagtccgggtcctcggtgaaggtctcctgcagggcttctggaggcaccttcggcaattatgctatcaactgggtgcgacaggcccctggacaagggcttgagtgggtgggaaggatcatccctgtctttgatacaacaaactacgcacagaagttccagggcagagtcacgattaccgcggacagatccacaaacacagccatcatgcaactgagcagtctgcgacctcaggacacggccatgtattattgtttgagaggttccacccgtggctgggatactgatggttttgatatctggggccaagggacaatggtcaccgtctcgagt

SEQ ID NO: 56 - DNA, 387 bp, homo sapiens
caggttcagctggtgcagtctggggctgtcgtggagacgcctggggcctcagtgaaggtctcctgcaaggcatctggatacatcttcggcaactactatatccactgggtgcggcaggcccctggacaagggcttgagtggatggcagttatcaatcccaatggtggtagcacaacttccgcacagaagttccaagacagaatcaccgtgaccagggacacgtccacgaccactgtctatttggaggttgacaacctgagatctgaggacacggccacatattattgtgcgagacagagatctgtaacagggggctttgacgcgtggcttttaatcccagatgcttctaatacctggggccaggggacaatggtcaccgtctcgagt

SEQ ID NO: 57 - DNA, 378 bp, homo sapiens
caggtgcagctggtgcagtctggggctgagatgaagaagcctgggtcctcggtgaaggtctcctgcaaggcttctggaggctccttcagcagctattctatcagctgggtgcgacaggcccctggacgagggcttgagtgggtgggaatgatcctgcctatctctggtacaacaaactacgcacagacatttcagggcagagtcatcattagcgcggacacatccacgagcacagcctacatggagctgaccagcctcacatctgaagacacggccgtgtatttctgtgcgagagtctttagagaatttagcacctcgacccttgacccctactactttgactactggggccagggaaccctggtcaccgtctcgagt

SEQ ID NO: 58 - DNA, 375 bp, homo sapiens
caggtgcagctggtggagtctgggggaggcgtggtccagcctgggaagtccgtgagactctcctgtgtaggctctggcttcaggctcatggactatgctatgcactgggtccgccaggctccaggcaagggactggattgggtggcagttatttcatatgatggagccaatgaatactacgcagagtccgtgaagggccgattcaccgtctccagagacaattcagacaacactctgtatctacaaatgaagagcctgagagctgaggacacggctgtgtatttctgtgcgagagcgggccgttcctctatgaatgaagaagttattatgtactttgacaactggggcctgggaaccctggtcaccgtctcgagt

SEQ ID NO: 59 - DNA, 381 bp, homo sapiens
gaggtgcagctgttggagtctgggggaggcttggtccagcctggggggtccctgagactctcctgtgtagcctccggattcacctttagtacctacgccatgacctgggtccgccaggctccagggaaggggctggagtgggtctcagtcattcgtgctagtggtgatagtgaaatctacgcagactccgtgaggggccggttcaccatctccagagacaattccaagaacacggtgtttctgcaaatggacagcctgagagtcgaggacacggccgtatatttctgtgcgaatataggccagcgtcggtattgtagtggtgatcactgctacggacactttgactactggggccagggaaccctggtcaccgtctcgagt

SEQ ID NO: 60 - DNA, 381 bp, homo sapiens
caggtgcagctggtggagtctgggggaggcgtggtccaacctgggaggtccctgagactctcctgtgcagcctctggattcggcttcaacacccatggcatgcactgggtccgccaggctccaggcaaggggctggagtggctgtcaattatctcacttgatgggattaagacccactatgcagactccgtgaagggccgattcaccatctccagagacaattccaagaacacggtgtttctacaattgagtggcctgagacctgaagacacggctgtatattactgtgcgaaagatcatattggggggacgaacgcatattttgaatggacagtcccgtttgacggctggggccagggaaccctggtcaccgtctcgagt

SEQ ID NO: 61 - DNA, 378 bp, homo sapiens
caggtcaccttgagggagtctggtccagcggtggtgaagcccacagaaacgctcactctgacctgcgccttctctgggttctcactcaacgccggtagagtgggtgtgagttggatccgtcagcccccagggcaggccccggaatggcttgcacgcattgattgggatgatgataaagcgttccgcacatctctgaagaccagactcagcatctccaaggactcctccaaaaaccaggtggtccttacactgagcaacatggaccctgcggacacagccacatattactgtgcccggacacaggtcttcgcaagtggaggctactacttgtactaccttgaccactggggccagggaaccctggtcaccgtctcgagt

SEQ ID NO: 62 - DNA, 387 bp, homo sapiens
caggtgcagctgcaggagtcgggcccaggactggtgaagccttcacagaccctgtccctcacctgcactgtctctagtggcgccatcagtggtgctgattactactggagttggatccgccagcccccagggaagggcctggagtgggttgggttcatctatgacagtgggagcacctactacaacccgtccctcaggagtcgagtgaccatatcaatagacacgtccaagaagcagttctccctgaagctgacctctgtgactgccgcagacacggccgtgtattactgtgccagagatctaggctacggtggtaactcttactcccactcctactactacggtttggacgtctggggccgagggaccacggtcaccgtctcgagt

SEQ ID NO: 63 - DNA, 357 bp, homo sapiens
caggtgcagctgcaggagtcgggcccaggactggtgaagccttcggagaccctgtccctcacctgcactgtctctggtggctccatcggaaattactactggggctggatccggcagcccccagggaagggacttgagtggattgggcatatctacttcggtggcaacaccaactacaacccttccctccagagtcgagtcaccatttcagtcgacacgtccaggaaccagttctccctgaagttgaactctgtgaccgccgcggacacggccgtgtattactgtgcgagggatagcagcaactggcccgcaggctatgaggactggggccagggaaccctggtcaccgtctcgagt

SEQ ID NO: 64 - DNA, 369 bp, homo sapiens
caggttcagctggtgcagtctggagctgaggtgaagaagcctggggcctcagtgaaggtctcctgcaaggtttctggttacacctttaccagtaatggtctcagctgggtgcgacaggcccctggacaagggtttgagtggctgggatggatcagcgctagtagtggaaacaaaaagtatgccccgaaattccagggaagagtcaccttgaccacagacatttccacgagcacagcctacatggaactgaggagtctgagatctgacgatacggccgtatattactgtgcgaaagatgggggcacctacgtgccctattctgatgcctttgatttctggggccaggggacaatggtcaccgtctcgagt

SEQ ID NO: 65 - DNA, 354 bp, homo sapiens
caggtccagctggtacagtctggggctgaggtgaagaagcctggggcctcagtgaaggtctcctgcagggtttccggacacactttcactgcattatccaaacactggatgcgacagggtcctggaggagggcttgagtggatgggattttttgatcctgaagatggtgacacaggctacgcacagaagttccagggcagagtcaccatgaccgaggacacagccacaggcacagcctacatggagctgagcagcctgacatctgacgacacggccgtatattattgtgcaacagtagcggcagctggaaactttgacaactggggccagggaaccctggtcaccgtctcgagt

SEQ ID NO: 66 - DNA, 378 bp, homo sapiens
caggtcaccttgaaggagtctggtcctgcgctggtgaaagccacacagaccctgacactgacctgcaccttctctgggttttcactcagtaggaatagaatgagtgtgagctggatccgtcagcccccagggaaggccctggagtggcttgcacgcattgattgggatgatgataaattctacaacacatctctgcagaccaggctcaccatctccaaggacacctccaaaaaccaggtggtccttacaatgaccaacatggaccctgtggacacagccacctattactgcgcacggactgggatatatgatagtagtggttattacctctactactttgactactggggccagggaaccctggtcaccgtctcgagt

SEQ ID NO: 67 - DNA, 390 bp, homo sapiens
caggtgcagctggtgcagtctggagctgaggtgaaggtgcctggggcctcagtgaaggtctcctgcaaggcttctggttacacctttaccacttacggtgtcagctgggtgcggcaggcccctggacaagggcttgagtggatgggttggatcagcgcttacaatggtaacacatactatctacagaagctccagggcagagtcaccatgaccacagacacatccacgagcacagcctacatggagctgcggggcctgaggtctgacgacacggccatgtattactgtgcgagagatcgtgttgggggcagctcgtccgaggttctatcgcgggccaaaaactacggtttggacgtctggggccaagggaccacggtcaccgtctcgagt

SEQ ID NO: 68 - DNA, 369 bp, homo sapiens
caggttcagctggtgcagtctggggctgaggtgaagaagcctggggcctcagttaaggtttcctgcaaggcttctgcaaacatcttcacttatgcaatgcattgggtgcgccaggcccccggacaaaggcttgagtggatgggatggatcaacgttggcaatggtcagacaaaatattcacagaggttccagggcagagtcaccattaccagggacacgtccgcgactacagcctacatggagctgagcaccctgagatctgaggacacggctgtgtattactgtgcgaggcgtgcgagccaatatggggaggtctatggcaactactttgactactggggccagggaaccctggtcaccgtctcgagt

SEQ ID NO: 69 - DNA, 378 bp, homo sapiens
caggtgcagctggtgcagtctggagctgaggtgaagaggcctggggcctcagtgaaggtctcctgcaaggcttcaggttacacctttatcagctatggtttcagctgggtgcgacaggcccctggacaagggcttgagtggatgggatggagcagcgtttacaatggtgacacaaactatgcacagaagttccacggcagagtcaacatgacgactgacacatcgacgaacacggcctacatggaactcaggggcctgagatctgacgacacggccgtgtatttctgtgcgagggatcgcaatgttgttctacttccagctgctccttttggaggtatggacgtctggggccaagggacaatggtcaccgtctcgagt

SEQ ID NO: 70 - DNA, 369 bp, homo sapiens
caggtgcagctggtggagtctgggggaggcgtggtccagccggggacttccctgagactctcctgtgcagcctctggattcaccttcagtacgtttggcatgcactgggtccgccaggctccaggcaaggggctggagtgggtggcagttatatcatatgatggaaataagaaatactatgcagactccgtgaagggccgattcaccatctccagagacaattccaagaacacgctgtatctgcaagtgaacagcctgagagtcgaggacacggctgtgtattactgtgcggcccaaactccatatttcaatgagagcagtgggttagtgccggactggggccagggcaccctggtcaccgtctcgagt

SEQ ID NO: 71 - DNA, 378 bp, homo sapiens
caggtgcagctggtgcagtctggagctgaggtgaagaagcctggggcctcagtgaaggtctcctgcaaggcttctggttacacctttatcagttttggcatcagctgggtgcgacaggcccctggacaaggacttgagtggatgggatggatcagcgcttacaatggtaacacagactatgcacagaggctccaggacagagtcaccatgactagagacacagccacgagcacagcctacttggagctgaggagcctgaaatctgacgacacggccgtgtactattgcactagagacgagtcgatgcttcggggagttactgaaggattcggacccattgactactggggccagggaaccctggtcaccgtctcgagt

SEQ ID NO: 72 - DNA, 384 bp, homo sapiens
gaagtgcagctggtgcagtctggagcagaggtgaaaaagccggggcagtctctgaagatctcctgtaagacttctggatacatctttaccaactactggatcggctgggtgcgccagaggcccgggaaaggcctggagtggatgggggtcatctttcctgctgactctgatgccagatacagcccgtcgttccaaggccaggtcaccatctcagccgacaagtccatcggtactgcctacctgcagtggagtagcctgaaggcctcggacaccgccatatattactgtgcgagaccgaaatattactttgatagtagtgggcaattctccgagatgtactactttgacttctggggccagggaaccctggtcaccgtctcgagt

SEQ ID NO: 73 - DNA, 357 bp, homo sapiens
caggttcagctggtgcagtctggacctgaggtgaagaagcctggggcctcagtgaaggtctcctgcaaggcttctggttatgtgttgaccaactatgccttcagctgggtgcggcaggcccctggacaagggcttgagtggctgggatggatcagcggctccaatggtaacacatactatgcagagaagttccagggccgagtcaccatgaccacagacacatccacgagcacagcctacatggagctgaggagtctgagatctgacgacacggccgtttatttctgtgcgagagatcttctgcggtccacttactttgactactggggccagggaaccctggtcaccgtctcgagt

SEQ ID NO: 74 - DNA, 378 bp, homo sapiens
caggtgcagctggtgcagtctggagctgaggtgaagaagcctggggcctcagtgaaggtctcctgcaaggcttctggttacaccttttccaactacggtttcagctgggtgcgacaggcccctggacgagggcttgagtggatgggatggatcagcgcttacaatggtaacacatactatgcacagaacctccagggcagagtcaccatgaccacagacacatccacgaccacagcctacatggtactgaggagcctgagatctgacgacacggccatgtattactgtgcgagagatggaaatacagcaggggttgatatgtggtcgcgtgatggttttgatatctggggccaggggacaatggtcaccgtctcgagt

SEQ ID NO: 75 - DNA, 393 bp, homo sapiens
gaggtgcagctgttggagtctgggggaggcttggtacagcctggggggcccctgaggctctcctgtgtagcctctggattcagctttagcagctatgccatgaactggatccgcctggctccagggaaggggctggagtgggtctcaggtattagtggtagcggtggtagcacttactacggagactccgtgaagggccggttcaccatctccagagacaattccaagaacacgctgtatctgcaaatgaacagcctgagagccgaggacacggccgtatattactgtgcgaaagagccgtggatcgatatagtagtggcatctgttatatccccctactactacgacggaatggacgtctggggccaagggaccacggtcaccgtctcgagt

SEQ ID NO: 76 - DNA, 369 bp, homo sapiens
caggttcagctggtgcagtctggggctgaggtgaagaagcctgggtcctcggtgaaggtctcctgcaaggcctctggaggatccttcgacggctacactatcagctggctgcgacaggcccctggacaggggcttgagtggatgggaagggtcgtccctacacttggttttccaaactacgcacagaagttccaaggcagagtcaccgttaccgcggacagatccaccaacacagcctacttggaattgagcagactgacatctgaagacacggccgtatattactgtgcgaggatgaatctcggatcgcatagcgggcgccccgggttcgacatgtggggccaaggaaccctggtcaccgtctcgagt

SEQ ID NO: 77 - DNA, 387 bp, homo sapiens
caggtgcagctggtggagtctgggggaggcgtggtccagcctgggaggtccttgagactctcctgtgcagtgtctggatccagcttcagtaaatatggcatacactgggtccgccaggctccaggcaaggggctggagtgggtggcagttatatcgtatgatggaagtaaaaagtatttcacagactccgtgaagggccgattcaccatcgccagagacaattcccagaacacggtttttctgcaaatgaacagcctgagagccgaggacacggctgtctattactgtgcgacaggagggggtgttaatgtcacctcgtggtccgacgtagagcactcgtcgtccttaggctactggggcctgggaaccctggtcaccgtctcgagt

SEQ ID NO: 78 - DNA, 375 bp, homo sapiens
caggtgcagctggtggagtctgggggaggcgtggtccagcctggggggtccctgagactctcctgtgcagcgtctggattcaccttcagtagctatggcatgcactgggtccgccaggctccaggcaaggggctggagtgggtggcatttatatggaatgatggaagtaataaatactatgcagactccgtgaagggccgattcaccatctccagagacaattccaagaacacgctgtatctgcaaatgaacagcctgagagctgaggacacggctgtgtattactgtgtgaaagatgaggtctatgatagtagtggttattacctgtactactttgactcttggggccagggaaccctggtcaccgtctcgagt

SEQ ID NO: 79 - DNA, 381 bp, homo sapiens
gaggtgcagctgttggagtctgggggaggcttggtacagcctggggggtccctgagactctcctgtgcagcctctggattcacgtttagctcctataccatgagctgggtccgccaggctccagggaaggggctggagtgggtctcaagtattagtgctagtactgttctcacatactacgcagactccgtgaagggccgcttcaccatctccagagacaattccaagaacacgctgtatctgcaaatgagtagcctgagagccgaggacacggccgtatattactgtgcgaaagattacgatttttggagtggctatcccgggggacagtactggttcttcgatctctggggccgtggcaccctggtcaccgtctcgagt

SEQ ID NO: 80 - DNA, 369 bp, homo sapiens
caggtgcagctgcaggagtcgggcccaggactggtgacgccttcggagaccctgtccgtcacttgcactgtctctaattattccatcgacaatgcttactactggggctggatccggcagcccccagggaagggtctggagtggataggcagtatccatcatagtgggagcgcctactacaattcgtccctcaagagtcgagccaccatatctatagacacgtccaagaaccaattctcgttgaacctgaggtctgtgaccgccgcagacacggccgtatattactgtgcgcgcgataccatcctcacgttcggggagccccactggttcgacccctggggccagggaaccctggtcaccgtctcgagt

SEQ ID NO: 81 - DNA, 372 bp, homo sapiens
caggtgcagctgcaggagtcgggcccaggactggtgaagccttcggagaccttgtccctcacctgcactgtctcaggtgactccatcagtaattactactggagttggatccggcagcccccagggaagggactggagtggattggagaaatatctaacacttggagcaccaattacaacccctccctcaagagtcgagtcaccatatctctagacatgcccaagaaccagttgtccctgaagctgagctctgtgaccgctgcggacacggccgtatattactgtgcgagagggcttttctatgacagtggtggttactacttgttttacttccaacactggggccagggcaccctggtcaccgtctcgagt

SEQ ID NO: 82 - DNA, 375 bp, homo sapiens
caggtgcagctggtggagtctgggggaggcgtggtccagcctgggaggtccctgagagtctcctgtgcagcgtctggattcaccttcagtaactatggcatgcactgggtccgccaggctccaggcaaggggctggagtgggtggcagttatatggtatgatgacagtaataaacagtatggagactccgtgaagggccgattcaccatctccagagacaattccaagagtacgctgtatctgcaaatggacagactgagagtcgaggacacggctgtgtattattgtgcgagagcctccgagtatagtatcagctggcgacacaggggggtccttgactactggggccagggaaccctggtcaccgtctcgagt

SEQ ID NO: 83 - DNA, 378 bp, homo sapiens
cagatcaccttgaaggagtctggtcctacgctggtgagacccacacagaccctcacactgacctgcaccttctctgggttctcactcagcactagtaaactgggtgtgggctggatccgtcagcccccaggaaaggccctggagtggcttgcactcgttgattgggatgatgataggcgctacaggccatctttgaagagcaggctcaccgtcaccaaggacacctccaaaaaccaggtggtccttacaatgaccaacatggaccctgtggacacagccacatattactgtgcacacagtgcctactatactagtagtggttattaccttcaatacttccatcactggggcccgggcaccctggtcaccgtctcgagt

SEQ ID NO: 84 - DNA, 375 bp, homo sapiens
gaggtgcagctggtggagtctgggggaggcgtggtacagcctggaggctccctgagactctcctgtgaagtctccggattcaccttcaatagttatgaaatgacctgggtccgccaggccccagggaaggggctggagtgggtttcacacattggtaatagtggttctatgatatactacgctgactctgtgaagggccgattcaccatctccagagacaacgccaagaactcactatatctgcaaatgaacagcctgagagtcgaggacacggctgtttattactgtgcgaggtcagattactatgatagtagtggttattatctcctctacttagactcctggggccatggaaccctggtcaccgtctcgagt

SEQ ID NO: 85 - DNA, 360 bp, homo sapiens
caggtgcagctggtgcagtctggggctgaggtgaggaagcctggggcctcagtgaaggtttcctgcaaggcttctggacatactttcattaactttgctatgcattgggtgcgccaggcccccggacaggggcttgagtggatgggatacatcaacgctgtcaatggtaacacacagtattcacagaagttccagggcagagtcacctttacgagggacacatccgcgaacacagcctacatggagctgagcagcctgagatctgaagacacggctgtgtattactgtgcgagaaacaatgggggctctgctatcattttttactactggggccagggaaccctggtcaccgtctcgagt

SEQ ID NO: 86 - DNA, 366 bp, homo sapiens
caggtgcagctggtggagtctgggggaggcgtggtccagcctgggaggtccctgagactctcctgtgcagcctctggattcagcttcagtagctatggcatgcactgggtccgccaggctccaggcaaggggctggagtgggtggcagttatatcaaatgatggaagtaataaatactatgcagactccgtgaagggccgattcaccatctccagagacaattccaagaaaacgatgtatctgcaaatgaacagcctgagagctgaggacacggctgtgtatttctgtgcgaagacaacagaccagcggctattagtggactggttcgacccctggggccagggaaccctggtcaccgtctcgagt

SEQ ID NO: 87 - DNA, 390 bp, homo sapiens
cagctgcagctgcaggagtcgggcccaggactggtgaagccatcggagaccctgtccctcacctgcactgcctctggtggctccatcaacagtagtaatttctactggggctggatccgccagcccccagggaaggggctggagtggattgggagtatcttttatagtgggaccacctactacaacccgtccctcaagagtcgagtcaccatatccgtagacacgtccaagaaccagttctccctgaagctgagccctgtgaccgccgcagacacggctgtctatcactgtgcgagacatggcttccggtattgtaataatggtgtatgctctataaatctcgatgcttttgatatctggggccaagggacaatggtcaccgtctcgagt

SEQ ID NO: 88 - DNA, 366 bp, homo sapiens
caggtgcagctggtggagtctgggggaggcgtcgtccagcctggaaagtccctgagactctcctgtgcagcgtctggattcagattcagtgactacggcatgcactgggtccggcaggctccaagcaaggggctggagtgggtggcagttatctggcatgacggaagtaatataaggtatgcagactccgtgaggggccgattttccatctccagagacaattccaagaacacgctgtatttgcaaatgaacagcatgagagccgacgacacggctttttattattgtgcgagagtcccgttccagatttggagtggtctttattttgaccactggggccagggaaccctggtcaccgtctcgagt

SEQ ID NO: 89 - protein, 216 aa, homo sapiens
EIVLTQSPATLSLSPGERATLSCRASQSVNSHLAWYQQKPGQAPRLLIYNTFNRVTGIPARFSGSGSGTDFTLTISSLATEDFGVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 215 aa, homo sapiens
DIQMTQSPSSLSASVGDRVTFTCRASQRISNHLNWYQQKPGKAPKLLIFGASTLQSGAPSRFSGSGSGTDFTLTITNVQPDDFATYYCQQSYRTPPINFGQGTRLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 217 aa, homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYDSSLSTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 213 aa, homo sapiens
DIQMTQSPSSLSASVGDRVTITCRASQSITGYLNWYQQKPGKAPKLLIYATSTLQSEVPSRFSGSGSGTDFTLTISSLQPEDFATYYCQQSYNTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 215 aa, homo sapiens
EIVLTQSPGTLSLSPGERATLSCRASQSVSSSYLAWYQQKPGQAPRLLIHGASTGATGTPDRFSGSGSGTDFTLTISTLEPEDFAVYYCQQYGRTPYTFGQGTKLENKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 219 aa, homo sapiens
DIVMTQTPLSLSVTPGQPASISCRSSQSLLRSDGKTFLYWYLQKPGQSPQPLMYEVSSRFSGVPDRFSGSGSGADFTLNISRVETEDVGIYYCMQGLKIRRTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 215 aa, homo sapiens
DIQMTQSPSTLSASVGDRVTFSCRASQSVSSWVAWYQQKPGKAPKLLISEASNLESGVPSRFSGSGSGTEFTLTISSLQPEDFATYYCQQYHSYSGYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 214 aa, homo sapiens
AIQLTQSPSSLSASVGDRVTLTCRASQGITDSLAWYQQKPGKAPKVLLYAASRLESGVPSRFSGRGSGTDFTLTISSLQPEDFATYYCQQYSKSPATFGPGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 219 aa, homo sapiens
DIVMTQSPLSLPVTPGEPASISCRSSQSLLNSNGFNYVDWYLQKPGQSPQLLIYLGSNRASGVPDRFSGSGSGTDFTLKISRVEAEDVGVYYCMQALETPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 215 aa, homo sapiens
EIVLTQSPGTLSLSPGGRATLSCRASQSVSSGYLAWYQQKPGQAPRLLIYGASGRATGIPDRFSGSGSGTDFTLTISRLEPEDFAVYYCQQYFGSPYTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 214 aa, homo sapiens
NIQMTQSPSAMSASVGDRVTITCRASQGISNYLVWFQQKPGKVPKRLIYAASSLQSGVPSRFSGSGSGTEFTLTISSLQPEDFATYYCLQHNISPYTFGQGTKLETKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 220 aa, homo sapiens
DIVMTQSPDSLAVSLGERATINCRSSETVLYTSKNQSYLAWYQQKARQPPKLLLYWASTRESGVPARFSGSGSGTDFTLAISSLQAEDVAVYYCQQFFRSPFTFGPGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 215 aa, homo sapiens
EIVLTQSPGTLSLSPGERVTLSCRASQSVSSSYIAWYQQKPGQAPRLVIYAASRRATGVPDRFSGSGSATDFTLTISRLEPEDLAVYYCQHYGNSLFTFGPGTKVDVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 215 aa, homo sapiens
DIQMTQSPSTLSASVGDRVTITCRASQSIGSRLAWYQQQPGKAPKFLIYDASSLESGVPSRFSGSGSGTEFTLTISSLQPEDLATYYCQQYNRDSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 219 aa, homo sapiens
DIVMTQSPLSLPVTPGEPASISCRSSQSLLHSDGRYYVDWYLQKPGQSPHLLIYLASNRASGVPDRFTGSGSGTDFTLKISRVEAEDVGVYYCMQGLHTPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 213 aa, homo sapiens
EIVMTQSPATLSASPGERATLSCWASQTIGGNLAWYQQKPGQAPRLLIYGASTRATGVPARFSGSGSGTEFTLAISSLQSEDFAVYYCQQYKNWYTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 215 aa, homo sapiens
DIQMTQSPSSLSASVGDRVTITCRASQTIASYVNWYQQKPGRAPSLLIYAASNLQSGVPPRFSGSGSGTDFTLTISGLQPDDFATYYCQQSYSYRALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 216 aa, homo sapiens
EIVLTQSPATLSLSPGERATLSCRASQSVSSSLAWYQQTPGQAPRLLIYDASYRVTGIPARFSGSGSGIDFTLTISSLEPEDFAVYYCQQRSNWPPGLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 214 aa, homo sapiens
AIQLTQSPSSLSASVGDTVTVTCRPSQDISSALAWYQQKPGKPPKLLIYGASTLDYGVPLRFSGTASGTHFTLTISSLQPEDFATYYCQQFNTYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 220 aa, homo sapiens
DIVMTQSPDSLAVSLGERATINCKSSQSVLYNSNNKNYLAWYQQKPGQPPKLLIHLASTREYGVPDRFSGSGSGTDFALIISSLQAEDVAVYYCQQYYQTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 214 aa, homo sapiens
DIQMTQSPSSLAASVGDRVTITCRASQFISSYLHWYQQRPGKAPKLLMYAASTLQSGVPSRFSGSGSGTDFTLTISSLQPEDFATYYCQQSYTNPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 214 aa, homo sapiens
DIQMTQSPSSLSASVGDRVTITCRASQSIASYLNWYQQKPGKAPKLLIYAASSLHSGVPSRFSGSGSGTDFTLTISSLQPEDFATYYCQHSYSTRFTFGPGTKVDVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 214 aa, homo sapiens
DIQMTQSPSTLSASVGDRVTITCRASQSVTSELAWYQQKPGKAPNFLIYKASSLESGVPSRFSGSGSGTEFTLTISSLQPDDFATYYCQQYNSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 214 aa, homo sapiens
DIQMTQSPSTLSASVGDRLTITCRASQNIYNWLAWYQQKPGKAPKLLIYDASTLESGVPSRFSGSGSGTEFTLTISSLQPDDFATYYCQQYNSLSPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 214 aa, homo sapiens
DIQLTQSPSFLSASLEDRVTITCRASQGISSYLAWYQQKPGKAPKLLLDAASTLQSGVPSRFSGSGSGTEFTLTISSLQPEDFATYYCQQLNSYPRTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 214 aa, homo sapiens
DIQMTQSPSSLSASVGDRVSITCRASQGISNYLAWYQQKPGKVPKLLIYAASTLQSGVPSRFSGSGSGTDFTLTISSLQPEDVATYYCQKYNSAPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 220 aa, homo sapiens
DIVMTQSPDSLAVSLGERATINCRSSQSVLYSSNNKNYLAWYQQKPGQPPKLLVYWASTRASGVPDRFSGSGSGTDFTLTLSSLQAEDVAVYYCQQFHSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 215 aa, homo sapiens
EIVLTQSPGTLSLSPGERATLSCRASQSVSSNYLAWYQQKPGQAPRLLIYGASSRAAGMPDRFSGSGSGTDFTLTISRLEPEDFAVYYCQQYGNSPLTFGGGTEVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 214 aa, homo sapiens
DIQMTQSPSSVSASVGDRVTITCRASQAISNWLAWYQQKPGKAPKLLIYAASSLQSGVPSRFSGSGSGTDFTLTISGLQPEDFATYYCQQADTFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 220 aa, homo sapiens
DIVMTQTPLSLPVTPGEPASISCRSSQSLLDSNDGNTYLDWYLQKPGQSPQLLIYTFSYRASGVPDRFSGSGSGTDFTLKISRVEAEDVGVYYCMQRIEFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 219 aa, homo sapiens
DIVMTQSPLSLPVTPGEPASISCRSSQSLLHRNEYNYLDWYLQKPGQSPQLLIYWGSNRASGVPDRFSGSGSGTDFTLKISRVEAEDVGVYYCMQTLQTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 214 aa, homo sapiens
DIQMTQSPSSVSASVGDRVTITCQASQDISNYLNWYQQKPGKAPKLLIFDATKLETGVPTRFIGSGSGTDFTVTITSLQPEDVATYYCQHFANLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 214 aa, homo sapiens
DIQMTQSPSSLSASVGDRVTITCRASQGIRNYLAWYQQKPGKVPKLLVFAASTLQSGVPSRFSGSGSGTDFTLTISSLQPEDVATYYCQRYNSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 215 aa, homo sapiens
DIQMTQSPSSLSASVGDRVTITCRASQIIASYLNWYQQKPGRAPKLLIYAASSLQSGVPSRFSGSGSGTDFTLTISSLQPEDFATYYCQQSYSTPIFTFGPGTKVNIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 213 aa, homo sapiens
EIVLTQSPATLSLSPGERATLSCRTSQSVSSYLAWYQQKPGQAPRLLIYDASNRATGIPARFSGSGSGTDFTLTISSLEPEDFAVYYCQQRSDWLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 215 aa, homo sapiens
EIVMTQSPATLSVSPGERATLSCRASQSIKNNLAWYQVKPGQAPRLLTSGASARATGIPGRFSGSGSGTDFTLTISSLQSEDIAVYYCQEYNNWPLLTFGGGTKVEIQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 215 aa, homo sapiens
DIQMTQSPPSLSASVGDRVTITCRASQRIASYLNWYQQKPGRAPKLLIFAASSLQSGVPSRFSGSGSGTDFTLTISSLQPEDYATYYCQQSYSTPIYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 214 aa, homo sapiens
DIQMTQSPSSLSASVGDRVTITCQASQGISNYLNWYQQKPGKAPKLLIFDASNLESEVPSRFSGRGSGTDFTFSISSLQPEDIATYFCQQYDNFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 214 aa, homo sapiens
DIQMTQSPSSLAASVGDRVTITCRASQTIASYVNWYQQKPGKAPNLLIYAASSLQSGVPSRFSGSGSGTDFTLTISSLQPEDFASYFCQQSYSFPYTFGQGTKLDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 215 aa, homo sapiens
DIQMTQSPSSLSASVGDRVTITCRASQTIASYVNWYQQKPGKAPKLLIYAASNLQSGVPSRFSGSGSGTDFTLTISSLQPEDFATYYCQQSYSVPRLTFGGGTKVDITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 213 aa, homo sapiens
DIQMTQSPSSLSASVGDRVTITCRSSQTISVFLNWYQQKPGKAPKLLIYAASSLHSAVPSRFSGSGSGTDFTLTISSLQPEDSATYYCQESFSSSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 215 aa, homo sapiens
EIVMTQSPATLSVSPGETATLSCRASQSVSSNLAWYQHKPGQAPRLLIHSASTRATGIPARFSGSGSGTEFTLTISSLQSEDFAVYYCQQYNMWPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 219 aa, homo sapiens
DIVMTQSPLSLPVTPGAPASISCRSSQSLLRTNGYNYLDWYLQKPGQSPQLLIYLGSIRASGVPDRFSGSGSGTDFTLKISRVEAEDVGVYYCMQSLQTSITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 214 aa, homo sapiens
EIVMTQSPATLSVSPGERATLSCRASQSVGNNLAWYQQRPGQAPRLLIYGASTRATGIPARFSGSGSGTEFTLTISSLQSEDFAVYYCQQYDKWPETFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - DNA, 648 bp, homo sapiens
gaaattgtgttgacacagtctccagccaccctgtccttgtctccaggagaaagagccaccctctcctgcagggccagtcagagtgttaacagccacttagcctggtaccaacagaaacctggccaggctcccaggctcctcatctataatacattcaatagggtcactggcatcccagccaggttcagtggcagtgggtctgggacagacttcactctcaccatcagcagccttgcgactgaagattttggcgtttattactgtcagcagcgtagcaactggcctcccgccctcactttcggcggagggaccaaagtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 134 - DNA, 645 bp, homo sapiens
gacatccagatgacccagtctccatcctccctgtctgcatctgtgggagacagagtcaccttcacttgccgggccagtcagaggattagcaaccatttaaattggtatcaacaaaagccagggaaagcccctaaactcctgatctttggtgcatccactcttcaaagtggggccccatcaaggttcagtggcagtggatctgggacagatttcactctcaccatcactaatgtacaacctgacgattttgcaacttactactgtcaacagagttacagaactcccccgatcaacttcggccaagggacacgcctggacattaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 135 - DNA, 651 bp, homo sapiens
gaaattgtgttgacgcagtctccaggcaccctgtctttgtctccaggggaaagagccaccctctcctgcagggccagtcagagtgttagcagcagctacttagcctggtatcagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatgatagctcactttctacgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 136 - DNA, 639 bp, homo sapiens
gacatccagatgacccagtctccatcctccctgtctgcatctgtaggagacagagtcaccatcacttgccgggcaagtcagagcattaccggctatttaaattggtatcagcagaaaccagggaaagcccctaaactcctgatctatgctacatccactttgcaaagtgaggtcccatcaaggttcagtggcagtggatctgggacagatttcactctcaccatcagcagtcttcaacctgaagattttgcaacttactactgtcaacagagttataataccctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 137 - DNA, 645 bp, homo sapiens
gaaattgtgttgacgcagtctccaggcaccctgtctttgtctccaggggaaagagccaccctctcctgcagggccagtcagagtgttagcagcagctacttagcctggtatcagcagaaacctggccaggctcccaggctcctcatacatggcgcatccaccggggccactggcaccccagacaggttcagtggcagtgggtctgggacagacttcactctcaccatcagtacactggagcctgaagattttgcagtgtattactgtcagcaatatggtaggacaccgtacacttttggccaggggaccaagctggagaacaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 138 - DNA, 657 bp, homo sapiens
gatattgtgatgacccagactccactctctctgtccgtcacccctggacagccggcctccatctcctgcaggtctagtcagagcctcctgcgaagtgatggaaagacgtttttgtattggtatctgcagaagccaggccagtctccccaacccctaatgtatgaggtgtccagccggttctctggagtgccagataggttcagtggcagcgggtcaggggcagatttcacactgaacatcagccgggtggagactgaggatgttgggatctattactgcatgcaaggtttgaaaattcgtcggacgtttggcccagggaccaaggtcgaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 139 - DNA, 645 bp, homo sapiens
gacatccagatgacccagtctccttccaccctgtctgcatctgtaggagacagagtcaccttctcttgccgggccagtcagagtgttagtagttgggtggcctggtatcagcagaaaccaggaaaagcccctaagctcctgatctctgaggcctccaatttggaaagtggggtcccatcccggttcagcggcagtggatccgggacagaattcactctcaccatcagcagcctgcagcctgaagattttgcaacttattactgccaacagtatcatagttactctgggtacacttttggccaggggaccaagttggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 140 - DNA, 642 bp, homo sapiens
gccatccagttgacccagtctccatcgtccctgtctgcatctgtaggcgacagagtcaccctcacttgccgggcgagtcagggcattaccgattctttagcctggtatcagcagaaaccagggaaagcccctaaggtcctgctctatgctgcttccagattggaaagtggggtcccatccaggttcagtggccgtggatctgggacggatttcactctcaccatcagcagcctgcagcctgaagactttgcaacttattactgtcaacagtattctaagtcccctgcgacgttcggcccagggaccaaggtggaaatcagacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 141 - DNA, 657 bp, homo sapiens
gatattgtgatgacccagtctccactctccctgcccgtcacccctggagagccggcctccatctcctgcaggtctagtcagagcctcctaaatagtaatggattcaactatgtggattggtacctgcagaagccagggcagtctccacaactcctgatctatttgggttctaatcgggcctccggggtccctgacaggttcagtggcagtggatcaggcacagattttacactgaaaatcagcagagtggaggctgaggatgttggggtttattactgcatgcaagctctagaaactccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 142 - DNA, 645 bp, homo sapiens
gaaattgtgttgacgcagtctccaggcaccctgtctttgtctccagggggaagagccaccctctcctgcagggccagtcagagtgttagcagcggctacttagcctggtaccagcagaaacctggccaggctcccaggctcctcatctatggtgcatccggcagggccactggcatcccagacaggttcagtggcagtgggtctgggacagacttcactctcaccatcagcagactggagcctgaagattttgcagtgtattactgtcagcagtattttggctcaccgtacacttttggccaggggaccaagctggagctcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 143 - DNA, 642 bp, homo sapiens
aacatccagatgacccagtctccatctgccatgtctgcatctgtaggagacagagtcaccatcacttgtcgggcgagtcagggcattagtaattatttagtctggtttcagcagaaaccagggaaagtccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatatttccccttacacttttggccaggggaccaagctggagaccaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 144 - DNA, 660 bp, homo sapiens
gacatcgtgatgacccagtctccagactccctggctgtgtctctgggcgagagggccaccatcaactgcaggtccagtgagactgttttatacacctctaaaaatcagagctacttagcttggtaccagcagaaagcacgacagcctcctaaactactcctttactgggcatctacccgggaatccggggtccctgcccgattcagtggcagcggatctgggacagatttcactctcgccatcagcagcctgcaggctgaagatgtggcagtttattactgtcagcaattttttaggagtcctttcactttcggccccgggaccagactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 145 - DNA, 645 bp, homo sapiens
gaaattgtgttgacgcagtctccaggcaccctgtctttgtctccaggggaaagagttaccctctcttgcagggccagtcagagtgttagcagcagttacatagcctggtaccagcagaagcctggccaggctcccaggctcgtcatctatgctgcatcccgcagggccactggcgtcccagacaggttcagtggcagtgggtctgcgacagacttcactctcaccatcagtagactggagcctgaagatcttgcagtgtattactgtcagcactatggtaactcactattcactttcggccctgggaccaaggtggatgtcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 146 - DNA, 645 bp, homo sapiens
gacatccagatgacccagtctccctccaccctgtctgcatctgtcggagacagagtcaccatcacttgccgggccagtcagagtattggtagccggttggcctggtatcagcagcaaccagggaaagcccctaaattcctgatctatgatgcctccagtttggaaagtggggtcccatcaaggttcagcggcagtggatcagggacagaattcactctcaccatcagcagcctgcagccggaggatcttgcaacttattactgccaacagtacaatagagattctccgtggacgttcggccaagggaccaaggtggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 147 - DNA, 657 bp, homo sapiens
gatattgtgatgacccagtctccactctccctgcccgtcaccccaggagagccggcctccatctcctgcaggtctagtcagagcctcctgcatagtgatggacgctactatgtggattggtacctgcagaagccagggcagtctccacacctcctgatctatttggcttctaatcgggcctccggggtccctgacaggttcactggcagtggatcaggcacagattttacactgaaaatcagcagagtggaggctgaggatgttggcgtttattactgcatgcaaggtctacacactccttggacgttcggccaggggaccaaggtggacatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 148 - DNA, 639 bp, homo sapiens
gaaattgtaatgacacagtctccagccaccctgtctgcgtccccaggggaaagagccaccctctcctgttgggccagtcagactattggaggcaacttagcctggtaccagcagaaacctggccaggctcccaggctcctcatctatggtgcatccaccagggccactggtgtcccagccaggttcagtggcagtgggtctgggacagagttcactctcgccatcagcagcctgcagtctgaagattttgcagtttattactgtcagcagtataaaaactggtacacttttggccaggggaccaagctggagctcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 149 - DNA, 645 bp, homo sapiens
gacatccagatgacccagtctccatcctccctgtctgcatctgtaggagacagagtcaccatcacttgccgggcaagtcagaccattgccagttacgtaaattggtaccaacaaaaaccagggagagcccctagtctcctgatctatgctgcatctaacttgcagagtggggtcccaccaaggttcagtggcagtggatctgggacagacttcactctcaccatcagcggtctgcaacctgacgattttgcaacttattactgtcaacagagttacagttatcgagcgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 150 - DNA, 648 bp, homo sapiens
gaaattgtgttgacacagtctccagccaccctgtcgttgtccccaggggaaagagccaccctctcctgcagggccagtcagagtgttagcagctccttagcctggtaccaacagacacctggccaggctcccaggcttctcatctatgatgcgtcctacagggtcactggcatcccagccaggttcagtggcagtgggtctgggatagacttcactctcaccatcagcagcctagagcctgaagattttgcagtttactattgtcagcagcgtagcaactggcctccggggctcactttcggcggggggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 151 - DNA, 642 bp, homo sapiens
gccatccagttgacccagtctccatcctccctgtctgcatctgttggagacacagtcaccgtcacttgccggccaagtcaggacattagcagtgctttagcctggtatcagcagaaaccagggaaacctcctaagctcctgatctatggtgcctccactttggattatggggtcccattaaggttcagcggcactgcatctgggacacatttcactctcaccatcagcagcctgcaacctgaagattttgcaacttattactgtcaacagtttaatacttacccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 152 - DNA, 660 bp, homo sapiens
gacatcgtgatgacccagtctccagactccctggctgtgtctctgggcgagagggccaccatcaactgcaagtccagccagagtgttttatacaactccaacaataagaactacttagcctggtatcagcagaaaccaggacagcctcctaagctcctcattcacttggcatctacccgggaatacggggtccctgaccgattcagtggcagcgggtctgggacagatttcgctctcatcatcagcagcctgcaggctgaagatgtggcagtttattactgtcaacaatattatcaaactcctctaacttttggccaggggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 153 - DNA, 642 bp, homo sapiens
gacatccagatgacccagtctccatcctccctggctgcatctgtaggagacagagtcaccatcacttgccgggcaagtcagttcattagcagctatttacattggtatcagcaaagaccaggcaaggcccctaaactcctgatgtatgctgcctccactttgcaaagtggggtcccatcaaggttcagtggcagtggatctgggacagatttcactctcaccatcagcagtctgcaacctgaagattttgcaacttactactgtcaacagagttacactaacccatacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 154 - DNA, 642 bp, homo sapiens
gacatccagatgacccagtctccatcctccctatctgcatctgtaggagacagagtcaccatcacttgccgggcaagtcagagcattgccagctatttaaattggtatcagcagaaaccagggaaagcccccaaactcctgatctatgctgcatccagtttgcatagtggggtcccatcaagattcagtggcagtggatctgggacagatttcactctcaccatcagcagtctgcaacctgaagattttgcaacttactactgtcaacacagttacagtactcgattcactttcggccctgggaccaaagtggatgtcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 155 - DNA, 642 bp, homo sapiens
gacatccagatgacccagtctccttcgaccctgtctgcatctgtaggagacagagtcaccatcacttgccgggccagtcagagtgttactagtgagttggcctggtatcagcagaaaccagggaaagcccctaacttcctgatctataaggcgtctagtttagaaagtggggtcccatcaaggttcagcggcagtggatctgggacagaattcactctcaccatcagcagcctgcagcctgatgattttgcaacttattactgccaacagtataatagttttccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 156 - DNA, 642 bp, homo sapiens
gacatccagatgacccagtctccttccaccctgtctgcatctgtaggcgacagactcaccatcacttgccgggccagtcagaatatttataactggttggcctggtatcagcagaaaccagggaaagcccctaaactcctgatctatgacgcctccactttggaaagtggggtcccatcaaggttcagcggcagtggatctgggacagagttcactctcaccatcagcagcctgcagcctgatgattttgcgacttattactgccaacaatataatagtttgtctccgacgttcggccaagggaccaaggtggaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 157 - DNA, 642 bp, homo sapiens
gacatccagttgacccagtctccatccttcctgtctgcatctttagaagacagagtcactatcacttgccgggccagtcagggcattagcagttatttagcctggtatcagcaaaaaccagggaaagcccctaagctcctgctcgatgctgcatccactttgcaaagtggggtcccatcaaggttcagcggcagtggatctgggacagagttcactctcacaatcagcagcctgcagcctgaagattttgcaacttattactgtcaacagcttaatagttaccctcggacgttcggccaagggaccaaggtggacatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 158 - DNA, 642 bp, homo sapiens
gacatccagatgacccagtctccatcctccctgtctgcatctgtaggagacagagtcagcatcacttgccgggcgagtcagggcattagcaattatttagcctggtatcagcagaaaccagggaaggttcctaagctcctgatctatgctgcatccactttgcaatcaggggtcccatctcggttcagtggcagtggatctgggacagatttcactctcaccatcagcagcctgcagcctgaggatgttgcaacttattactgtcaaaagtataacagtgcccctcaaacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 159 - DNA, 660 bp, homo sapiens
gacatcgtgatgacccagtctccagactccctggctgtgtctctgggcgagagggccaccatcaactgcaggtccagccagagtgttttatacagctccaacaataagaactacttagcttggtaccagcagaaaccaggacagcctcctaagctgctcgtttactgggcatcaacccgggcatccggggtccctgaccgattcagtggcagcgggtctgggacagatttcactctcaccctcagcagcctgcaggctgaagatgtggcagtttattactgtcagcagtttcatagtactcctcggacgttcggccaagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 160 - DNA, 645 bp, homo sapiens
gaaattgtgttgacgcagtctccaggcaccctgtctttgtctccaggggaaagagccaccctctcctgcagggccagtcagagtgttagcagcaactacttagcctggtaccagcagaaacctggccaggctcccaggctcctcatctatggtgcatccagcagggccgctggcatgccagacaggttcagtggcagtgggtctgggacagacttcactctcaccatcagcagactggagcctgaagattttgcagtgtattactgtcagcagtatggtaactcaccgctcactttcggcggagggaccgaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 161 - DNA, 642 bp, homo sapiens
gacatccagatgacccagtctccatcttctgtgtctgcatctgtaggagacagagtcaccatcacttgtcgggcgagtcaggctattagtaactggttagcctggtatcagcagaaaccaggaaaagcccctaagctcctgatctatgctgcatccagtttgcaaagtggggtcccatcaagattcagcggcagtggatctgggacagatttcactctcactatcagcggcctgcagcctgaggattttgcaacttactattgtcaacaggctgacactttccctttcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 162 - DNA, 660 bp, homo sapiens
gatattgtgatgacccagactccactctccctgcccgtcacccctggagagccggcctccatctcctgcaggtctagtcagagcctcttggatagtaatgatggaaacacctatttggactggtacctgcagaagccagggcagtctccacagctcctgatttatacattttcctatcgggcctctggagtcccagacaggttcagtggcagtgggtctggcactgatttcacactgaaaatcagcagggtggaggccgaggatgttggagtttattactgcatgcaacgtatcgagtttccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 163 - DNA, 657 bp, homo sapiens
gatattgtgatgacccagtctccactctccctgcccgtcacccctggagagccggcctccatctcctgcaggtctagtcagagcctcctgcatagaaatgagtacaactatttggattggtacttgcagaagccagggcagtctccacagctcctgatctattggggttctaatcgggcctccggggtccctgacaggttcagtggcagtggatcaggcacagattttacactgaaaatcagcagagtggaggctgaggatgttggggtttattactgcatgcaaactctacaaactcctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 164 - DNA, 642 bp, homo sapiens
gacatccagatgacccagtctccatcctccgtgtctgcatctgtgggagacagagtcaccatcacttgccaggcgagtcaagacattagcaactatttaaattggtatcagcagaaaccagggaaagcccctaagctcctgatcttcgatgcaaccaaattggagacaggggtcccaacaaggttcattggaagtggatctgggacagattttactgtcaccatcaccagcctgcagcctgaagatgttgcaacatattactgtcaacactttgctaatctcccatacacttttggccaggggaccaagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 165 - DNA, 642 bp, homo sapiens
gacatccagatgacccagtctccatcttccctgtctgcatctgtaggagacagagtcaccatcacttgccgggcgagtcagggcattaggaattatttagcctggtatcagcagaaaccagggaaagttcctaagctcctggtctttgctgcatccactttgcaatcaggggtcccatctcggttcagtggcagtggatctgggacagatttcactctcaccatcagcagcctgcagcctgaggatgttgcaacttattactgtcaaaggtataacagtgccccgctcactttcggcggagggacgaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 166 - DNA, 645 bp, homo sapiens
gacatccagatgacccagtctccatcctccctgtctgcatctgtaggagacagagtcaccatcacttgccgggcaagtcagatcattgccagctatttaaattggtatcagcagaaaccaggcagagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccccatattcactttcggccctgggaccaaggtgaatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 167 - DNA, 639 bp, homo sapiens
gaaattgtgttgacacagtctccagccaccctgtctttgtctccaggggaaagagccaccctctcctgcaggaccagtcagagtgttagcagctacttagcctggtaccaacagaaacctggccaggctcccaggctcctcatctatgatgcttccaatagggccactggcatcccagccaggttcagtggcagtgggtctgggacagacttcactctcaccatcagcagcctagagcctgaagattttgcagtttattactgtcagcagcgtagtgactggctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 168 - DNA, 645 bp, homo sapiens
gaaattgtaatgacacagtctccagccaccctgtctgtgtctccaggggaaagagccaccctctcctgcagggccagtcagagtattaaaaacaacttggcctggtaccaggtgaaacctggccaggctcccaggctcctcacctctggtgcatccgccagggccactggaattccaggcaggttcagtggcagtgggtctgggactgacttcactctcaccatcagcagcctccagtctgaagatattgcagtttattactgtcaggagtataataattggcccctgctcactttcggcggagggaccaaggtggagatccaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 169 - DNA, 645 bp, homo sapiens
gacatccagatgacccagtctcctccctccctgtctgcatctgtgggagacagagtcaccatcacttgccgggcaagtcagaggattgccagctatttaaattggtatcagcagaaaccagggagagcccctaagctcctgatctttgctgcatccagtttacaaagtggggtcccatcaaggttcagtggcagtggatctgggacagacttcactctcaccatcagtagtctgcaacctgaagattatgcgacttactactgtcaacagagttacagtactcccatctacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 170 - DNA, 642 bp, homo sapiens
gacatccagatgacccagtctccatcctccctgtctgcatctgtaggagacagagtcaccatcacttgccaggcgagtcagggcattagcaactatttaaattggtatcaacagaaaccagggaaagcccctaagctcctgatcttcgatgcatccaatttggaatcagaggtcccatcaaggttcagtggacgtggatctgggacagattttactttctccatcagcagcctgcagcctgaagatattgcaacatatttctgtcaacagtatgataatttcccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 171 - DNA, 642 bp, homo sapiens
gacatccagatgacccagtctccatcctccctggctgcatctgtaggagacagagtcaccatcacctgccgggcaagtcagacgattgccagttatgtaaattggtatcaacagaaaccagggaaagcccctaatctcctgatctatgctgcatccagtttgcaaagtggggtcccatcaaggttcagtggcagtggatctgggacagatttcactctcaccatcagcagtctgcaacctgaagattttgcatcttacttctgtcaacagagttacagtttcccgtacacttttggccaggggaccaagctggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 172 - DNA, 645 bp, homo sapiens
gacatccagatgacccagtctccatcctccctgtctgcatctgtaggagacagagtcaccatcacttgccgggcaagtcagaccattgccagctatgtaaattggtatcagcagaaaccagggaaagcccctaagctcctgatctatgctgcatccaatttgcaaagtggggtcccttcaaggttcagtggcagtggatctgggacagatttcactctcaccatcagcagtctgcaacctgaagattttgcaacttactactgtcaacagagttacagtgtccctcggctcactttcggcggagggaccaaggtggacatcacacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 173 - DNA, 639 bp, homo sapiens
gacatccagatgacccagtctccatcctccctgtctgcatctgtaggagacagagtcaccatcacttgccggtcaagtcagaccattagcgtctttttaaattggtatcagcagaaaccagggaaagcccctaagctcctgatctatgccgcatccagtttgcacagtgcggtcccatcaaggttcagtggcagtggatctgggacagatttcactctcaccatcagcagtctgcaacctgaagattctgcaacttactactgtcaagagagtttcagtagctcaactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 174 - DNA, 645 bp, homo sapiens
gaaattgtaatgacacagtctccagccaccctgtctgtgtctccaggggaaacagccaccctctcctgcagggccagtcagagtgttagcagcaacttagcctggtaccaacataaacctggccaggctcccaggctcctcatccatagtgcatccaccagggccactgggatcccagccaggttcagtggcagtgggtctgggacagagttcactctcaccataagcagcctgcagtctgaagattttgcagtttattactgtcagcagtataatatgtggcctccctggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 175 - DNA, 657 bp, homo sapiens
gatattgtgatgacccagtctccactctccctgcccgtcacccctggagcgccggcctccatctcctgcaggtctagtcagagcctcctgcgtactaatggatacaactatttggattggtacctgcagaagccagggcagtctccacagctcctgatctatttgggttctattcgggcctccggggtccctgacaggttcagtggcagtggctcaggcacagattttacactgaaaatcagcagagtggaggctgaggatgttggggtttattactgcatgcaatctctacaaacttcgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 176 - DNA, 642 bp, homo sapiens
gaaattgtaatgacacagtctccagccaccctgtctgtgtctccgggggaaagagccaccctctcctgcagggctagtcagagtgttggcaacaacttagcctggtaccagcagagacctggccaggctcccagactcctcatctatggtgcgtccaccagggccactggtatcccagccaggttcagtggcagtgggtctgggacagagttcactctcaccatcagcagcctgcagtctgaggattttgcagtttattactgtcagcagtatgataagtggcctgagacgttcggccaggggaccaaggtggacatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 177 - DNA, 1602 bp, Homo sapiens
agtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 178 - protein, 331 aa, Homo sapiens
SASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - DNA, 20 bp, artificial sequence
  Synthetic DNA primer
gacngatgggcccttggtgg

SEQ ID NO: 180 - DNA, 20 bp, artificial sequence
  Synthetic DNA primer
gagtggctcctgggggaaga

SEQ ID NO: 181 - DNA, 36 bp, artificial sequence
  Synthetic DNA primer
tattcccatggcgcgcccagntgcagctggtgcant

SEQ ID NO: 182 - DNA, 36 bp, artificial sequence
  Synthetic DNA primer
tattcccatggcgcgccnaggtccagctggtncagt

SEQ ID NO: 183 - DNA, 36 bp, artificial sequence
  Synthetic DNA primer
tattcccatggcgcgcccagntcaccttgaaggagt

SEQ ID NO: 184 - DNA, 35 bp, artificial sequence
  Synthetic DNA primer
tattcccatggcgcgccnaggtgcagctggtggag

SEQ ID NO: 185 - DNA, 36 bp, artificial sequence
  Synthetic DNA primer
tattcccatggcgcgcccaggtgcagctacagcagt

SEQ ID NO: 186 - DNA, 36 bp, artificial sequence
  Synthetic DNA primer
tattcccatggcgcgcccagntgcagctgcaggagt

SEQ ID NO: 187 - DNA, 36 bp, artificial sequence
  Synthetic DNA primer
tattcccatggcgcgccgangtgcagctggtgcagt

SEQ ID NO: 188 - DNA, 37 bp, artificial sequence
  Synthetic DNA primer
tattcccatggcgcgcccaggtacagctgcagcagtc

SEQ ID NO: 189 - DNA, 38 bp, artificial sequence
  Synthetic DNA primer
atatatatgcggccgcttattaacactctcccctgttg

SEQ ID NO: 190 - DNA, 45 bp, artificial sequence
  Synthetic DNA primer
ggcgcgccatgggaatagctagccgacatccagntgacccagtct

SEQ ID NO: 191 - DNA, 45 bp, artificial sequence
  Synthetic DNA primer
ggcgcgccatgggaatagctagccgatgttgtgatgactcagtct

SEQ ID NO: 192 - DNA, 45 bp, artificial sequence
  Synthetic DNA primer
ggcgcgccatgggaatagctagccgaaattgtgntgacncagtct

SEQ ID NO: 193 - DNA, 45 bp, artificial sequence
  Synthetic DNA primer
ggcgcgccatgggaatagctagccgatattgtgatgacccacact

SEQ ID NO: 194 - DNA, 43 bp, artificial sequence
  Synthetic DNA primer
ggcgcgccatgggaatagctagccgaaacgacactcacgcagt

SEQ ID NO: 195 - DNA, 45 bp, artificial sequence
  Synthetic DNA primer
ggcgcgccatgggaatagctagccgaaattgtgctgactcagtct

SEQ ID NO: 196 - DNA, 51 bp, artificial sequence
  Synthetic DNA primer
accgcctccaccggcggccgcttattaacactctcccctgttgaagctctt

SEQ ID NO: 197 - DNA, 30 bp, artificial sequence
  Synthetic DNA primer
ggaggcgctcgagacggtgaccagggtgcc

SEQ ID NO: 198 - DNA, 30 bp, artificial sequence
  Synthetic DNA primer
ggaggcgctcgagacggtgaccattgtccc

SEQ ID NO: 199 - DNA, 30 bp, artificial sequence
  Synthetic DNA primer
ggaggcgctcgagacggtgaccagggttcc

SEQ ID NO: 200 - DNA, 30 bp, artificial sequence
  Synthetic DNA primer
ggaggcgctcgagacggtgaccgtggtccc

SEQ ID NO: 201 - protein, 5 aa, Homo sapiens
DYDWS

SEQ ID NO: 202 - protein, 5 aa, Homo sapiens
TYGMH

SEQ ID NO: 203 - protein, 5 aa, Homo sapiens
TYALT

SEQ ID NO: 204 - protein, 5 aa, Homo sapiens
GYYMH

SEQ ID NO: 205 - protein, 5 aa, Homo sapiens
DYYMS

SEQ ID NO: 206 - protein, 5 aa, Homo sapiens
NYGLN

SEQ ID NO: 207 - protein, 7 aa, Homo sapiens
SGDYYWS

SEQ ID NO: 208 - protein, 5 aa, Homo sapiens
HFGMH

SEQ ID NO: 209 - protein, 5 aa, Homo sapiens
RFGIS

SEQ ID NO: 210 - protein, 5 aa, Homo sapiens
SYVMN

SEQ ID NO: 211 - protein, 5 aa, Homo sapiens
NYGMH

SEQ ID NO: 212 - protein, 5 aa, Homo sapiens
DYGMN

SEQ ID NO: 213 - protein, 5 aa, Homo sapiens
SYAMH

SEQ ID NO: 214 - protein, 5 aa, Homo sapiens
SYEMN

SEQ ID NO: 215 - protein, 7 aa, Homo sapiens
SGDYFWS

SEQ ID NO: 216 - protein, 5 aa, Homo sapiens
NYAMH

SEQ ID NO: 217 - protein, 5 aa, Homo sapiens
GDFWS

SEQ ID NO: 218 - protein, 5 aa, Homo sapiens
SYWIG

SEQ ID NO: 219 - protein, 7 aa, Homo sapiens
TTRMSVS

SEQ ID NO: 220 - protein, 7 aa, Homo sapiens
FVSTWIG

SEQ ID NO: 221 - protein, 5 aa, Homo sapiens
NYAIN

SEQ ID NO: 222 - protein, 5 aa, Homo sapiens
NYYIH

SEQ ID NO: 223 - protein, 5 aa, Homo sapiens
SYSIS

SEQ ID NO: 224 - protein, 5 aa, Homo sapiens
SYWIG

SEQ ID NO: 225 - protein, 5 aa, Homo sapiens
DYAMH

SEQ ID NO: 226 - protein, 5 aa, Homo sapiens
TYAMT

SEQ ID NO: 227 - protein, 5 aa, Homo sapiens
THGMH

SEQ ID NO: 228 - protein, 7 aa, Homo sapiens
AGRVGVS

SEQ ID NO: 229 - protein, 7 aa, Homo sapiens
GADYYWS

SEQ ID NO: 230 - protein, 5 aa, Homo sapiens
NSWIG

SEQ ID NO: 231 - protein, 6 aa, Homo sapiens
SGHFWG

SEQ ID NO: 232 - protein, 5 aa, Homo sapiens
NYYWG

SEQ ID NO: 233 - protein, 5 aa, Homo sapiens
SNGLS

SEQ ID NO: 234 - protein, 5 aa, Homo sapiens
ALSKH

SEQ ID NO: 235 - protein, 5 aa, Homo sapiens
TNGLH

SEQ ID NO: 236 - protein, 7 aa, Homo sapiens
RNRMSVS

SEQ ID NO: 237 - protein, 5 aa, Homo sapiens
TYGVS

SEQ ID NO: 238 - protein, 4 aa, Homo sapiens
YAMH

SEQ ID NO: 239 - protein, 5 aa, Homo sapiens
YIGMH

SEQ ID NO: 240 - protein, 5 aa, Homo sapiens
TYGLN

SEQ ID NO: 241 - protein, 5 aa, Homo sapiens
SYGFS

SEQ ID NO: 242 - protein, 6 aa, Homo sapiens
SGHYWG

SEQ ID NO: 243 - protein, 5 aa, Homo sapiens
TFGMH

SEQ ID NO: 244 - protein, 5 aa, Homo sapiens
SYGLH

SEQ ID NO: 245 - protein, 5 aa, Homo sapiens
SFGIS

SEQ ID NO: 246 - protein, 5 aa, Homo sapiens
RYGIS

SEQ ID NO: 247 - protein, 5 aa, Homo sapiens
NSGVS

SEQ ID NO: 248 - protein, 5 aa, Homo sapiens
SYGIS

SEQ ID NO: 249 - protein, 7 aa, Homo sapiens
SGGYSWS

SEQ ID NO: 250 - protein, 7 aa, Homo sapiens
SDKNYWS

SEQ ID NO: 251 - protein, 5 aa, Homo sapiens
GSTMH

SEQ ID NO: 252 - protein, 5 aa, Homo sapiens
TYTLH

SEQ ID NO: 253 - protein, 5 aa, Homo sapiens
SLGFS

SEQ ID NO: 254 - protein, 5 aa, Homo sapiens
GYTIH

SEQ ID NO: 255 - protein, 5 aa, Homo sapiens
NYWIG

SEQ ID NO: 256 - protein, 5 aa, Homo sapiens
NYAFS

SEQ ID NO: 257 - protein, 5 aa, Homo sapiens
NYGFS

SEQ ID NO: 258 - protein, 5 aa, Homo sapiens
SYAMN

SEQ ID NO: 259 - protein, 5 aa, Homo sapiens
GYTIS

SEQ ID NO: 260 - protein, 5 aa, Homo sapiens
KYGIH

SEQ ID NO: 261 - protein, 5 aa, Homo sapiens
SYGMH

SEQ ID NO: 262 - protein, 5 aa, Homo sapiens
SYTMS

SEQ ID NO: 263 - protein, 5 aa, Homo sapiens
TYGIS

SEQ ID NO: 264 - protein, 5 aa, Homo sapiens
RYTIH

SEQ ID NO: 265 - protein, 6 aa, Homo sapiens
NAYYWG

SEQ ID NO: 266 - protein, 5 aa, Homo sapiens
YYAMH

SEQ ID NO: 267 - protein, 5 aa, Homo sapiens
NYYWS

SEQ ID NO: 268 - protein, 5 aa, Homo sapiens
NYGMH

SEQ ID NO: 269 - protein, 5 aa, Homo sapiens
HYGMH

SEQ ID NO: 270 - protein, 5 aa, Homo sapiens
AYAMS

SEQ ID NO: 271 - protein, 7 aa, Homo sapiens
TSKLGVG

SEQ ID NO: 272 - protein, 5 aa, Homo sapiens
SYEMT

SEQ ID NO: 273 - protein, 5 aa, Homo sapiens
NFAMH

SEQ ID NO: 274 - protein, 6 aa, Homo sapiens
SNYYWG

SEQ ID NO: 275 - protein, 5 aa, Homo sapiens
SYGMH

SEQ ID NO: 276 - protein, 7 aa, Homo sapiens
TSRMSVS

SEQ ID NO: 277 - protein, 7 aa, Homo sapiens
SSNFYWG

SEQ ID NO: 278 - protein, 5 aa, Homo sapiens
TYGIS

SEQ ID NO: 279 - protein, 5 aa, Homo sapiens
KFYIH

SEQ ID NO: 280 - protein, 5 aa, Homo sapiens
SYTMH

SEQ ID NO: 281 - protein, 5 aa, Homo sapiens
NAWMS

SEQ ID NO: 282 - protein, 5 aa, Homo sapiens
IYGMH

SEQ ID NO: 283 - protein, 5 aa, Homo sapiens
DYGMH

SEQ ID NO: 284 - protein, 6 aa, Homo sapiens
SEYYWG

SEQ ID NO: 285 - protein, 5 aa, Homo sapiens
DYCMH

SEQ ID NO: 286 - protein, 16 aa, Homo sapiens
NINYRGNTNYNPSLKS

SEQ ID NO: 287 - protein, 17 aa, Homo sapiens
FIRYDGSTQDYVDSVKG

SEQ ID NO: 288 - protein, 17 aa, Homo sapiens
RITPMFDITNYAQKFQG

SEQ ID NO: 289 - protein, 17 aa, Homo sapiens
WINTSSGGTNYAQKFQG

SEQ ID NO: 290 - protein, 17 aa, Homo sapiens
YINRGGTTIYYADSVKG

SEQ ID NO: 291 - protein, 17 aa, Homo sapiens
WINAYNDNTYYSPSLQG

SEQ ID NO: 292 - protein, 16 aa, Homo sapiens
YIFHSGTTYYNPSLKS

SEQ ID NO: 293 - protein, 17 aa, Homo sapiens
IISYDGNNVHYADSVKG

SEQ ID NO: 294 - protein, 17 aa, Homo sapiens
WISADNGNTYYAQNFQD

SEQ ID NO: 295 - protein, 17 aa, Homo sapiens
WINTNTGDPAYAQDFTG

SEQ ID NO: 296 - protein, 17 aa, Homo sapiens
VISYDGRNKYFADSVKG

SEQ ID NO: 297 - protein, 17 aa, Homo sapiens
VIWHDGSNKNYLDSVKG

SEQ ID NO: 298 - protein, 17 aa, Homo sapiens
VIYYEGSNEYYADSVKG

SEQ ID NO: 299 - protein, 17 aa, Homo sapiens
YIGTGGSDIYYGDSVKG

SEQ ID NO: 300 - protein, 16 aa, Homo sapiens
YIYSSGSTFYNASLKS

SEQ ID NO: 301 - protein, 17 aa, Homo sapiens
ATSTDGGSTYYADSLKG

SEQ ID NO: 302 - protein, 16 aa, Homo sapiens
YIYYRGSTYYNPSLKS

SEQ ID NO: 303 - protein, 17 aa, Homo sapiens
IVYPGDSDTTYSPSFQG

SEQ ID NO: 304 - protein, 16 aa, Homo sapiens
RIDWDDDKYYSTSLKT

SEQ ID NO: 305 - protein, 17 aa, Homo sapiens
IINPADSDTRYSPSFQG

SEQ ID NO: 306 - protein, 17 aa, Homo sapiens
RIIPVFDTTNYAQKFQG

SEQ ID NO: 307 - protein, 17 aa, Homo sapiens
VINPNGGSTTSAQKFQD

SEQ ID NO: 308 - protein, 17 aa, Homo sapiens
MILPISGTTNYAQTFQG

SEQ ID NO: 309 - protein, 17 aa, Homo sapiens
IIYPGDSDTRNSPSFQG

SEQ ID NO: 310 - protein, 17 aa, Homo sapiens
VISYDGANEYYAESVKG

SEQ ID NO: 311 - protein, 17 aa, Homo sapiens
VIRASGDSEIYADSVRG

SEQ ID NO: 312 - protein, 17 aa, Homo sapiens
IISLDGIKTHYADSVKG

SEQ ID NO: 313 - protein, 16 aa, Homo sapiens
RIDWDDDKAFRTSLKT

SEQ ID NO: 314 - protein, 16 aa, Homo sapiens
FIYDSGSTYYNPSLRS

SEQ ID NO: 315 - protein, 17 aa, Homo sapiens
IIYPGDSTTTYTPSFQG

SEQ ID NO: 316 - protein, 16 aa, Homo sapiens
SIFHSGTTFHNPSLKS

SEQ ID NO: 317 - protein, 16 aa, Homo sapiens
HIYFGGNTNYNPSLQS

SEQ ID NO: 318 - protein, 17 aa, Homo sapiens
WISASSGNKKYAPKFQG

SEQ ID NO: 319 - protein, 17 aa, Homo sapiens
FFDPEDGDTGYAQKFQG

SEQ ID NO: 320 - protein, 17 aa, Homo sapiens
LINAGNGDTRFSQKFQG

SEQ ID NO: 321 - protein, 16 aa, Homo sapiens
RIDWDDDKFYNTSLQT

SEQ ID NO: 322 - protein, 17 aa, Homo sapiens
WISAYNGNTYYLQKLQG

SEQ ID NO: 323 - protein, 17 aa, Homo sapiens
WINVGNGQTKYSQRFQG

SEQ ID NO: 324 - protein, 17 aa, Homo sapiens
AISYDGSNKQYADSVKG

SEQ ID NO: 325 - protein, 17 aa, Homo sapiens
WVSAHNGNTYYAEKFHD

SEQ ID NO: 326 - protein, 17 aa, Homo sapiens
WSSVYNGDTNYAQKFHG

SEQ ID NO: 327 - protein, 16 aa, Homo sapiens
SIYDSGNTYYTPSLKS

SEQ ID NO: 328 - protein, 17 aa, Homo sapiens
VISYDGNKKYYADSVKG

SEQ ID NO: 329 - protein, 17 aa, Homo sapiens
EISYDGGSKFYTDSVKG

SEQ ID NO: 330 - protein, 17 aa, Homo sapiens
WISAYNGNTDYAQRLQD

SEQ ID NO: 331 - protein, 17 aa, Homo sapiens
WISAYNGNTYYAQNLQG

SEQ ID NO: 332 - protein, 17 aa, Homo sapiens
WISAYNGNTYYRQSLQD

SEQ ID NO: 333 - protein, 17 aa, Homo sapiens
WIGTDNGNTYYAQKFQG

SEQ ID NO: 334 - protein, 16 aa, Homo sapiens
YIYHSGSTYYNPSLKS

SEQ ID NO: 335 - protein, 16 aa, Homo sapiens
RLYPSGNTDYHPSLKS

SEQ ID NO: 336 - protein, 19 aa, Homo sapiens
RIRSKANSYATEYAASVKG

SEQ ID NO: 337 - protein, 17 aa, Homo sapiens
LINAANGHTKYSQRFQG

SEQ ID NO: 338 - protein, 17 aa, Homo sapiens
WTSAHNGNTYYAEEFQD

SEQ ID NO: 339 - protein, 17 aa, Homo sapiens
RLVPSLNIPNYAQKFQG

SEQ ID NO: 340 - protein, 17 aa, Homo sapiens
VIFPADSDARYSPSFQG

SEQ ID NO: 341 - protein, 17 aa, Homo sapiens
WISGSNGNTYYAEKFQG

SEQ ID NO: 342 - protein, 17 aa, Homo sapiens
WISAYNGNTYYAQNLQG

SEQ ID NO: 343 - protein, 17 aa, Homo sapiens
GISGSGGSTYYGDSVKG

SEQ ID NO: 344 - protein, 17 aa, Homo sapiens
RVVPTLGFPNYAQKFQG

SEQ ID NO: 345 - protein, 17 aa, Homo sapiens
VISYDGSKKYFTDSVKG

SEQ ID NO: 346 - protein, 17 aa, Homo sapiens
FIWNDGSNKYYADSVKG

SEQ ID NO: 347 - protein, 17 aa, Homo sapiens
SISASTVLTYYADSVKG

SEQ ID NO: 348 - protein, 17 aa, Homo sapiens
WISADNGNTYYAQKFQG

SEQ ID NO: 349 - protein, 17 aa, Homo sapiens
RVVPSLGIPNYAPKFQG

SEQ ID NO: 350 - protein, 16 aa, Homo sapiens
SIHHSGSAYYNSSLKS

SEQ ID NO: 351 - protein, 17 aa, Homo sapiens
VISYGETNKLYADSVKG

SEQ ID NO: 352 - protein, 16 aa, Homo sapiens
EISNTWSTNYNPSLKS

SEQ ID NO: 353 - protein, 17 aa, Homo sapiens
VIWYDDSNKQYGDSVKG

SEQ ID NO: 354 - protein, 17 aa, Homo sapiens
VISHDGNIKYSADSVKG

SEQ ID NO: 355 - protein, 17 aa, Homo sapiens
AISGGGGTTYYADSVKG

SEQ ID NO: 356 - protein, 16 aa, Homo sapiens
LVDWDDDRRYRPSLKS

SEQ ID NO: 357 - protein, 17 aa, Homo sapiens
HIGNSGSMIYYADSVKG

SEQ ID NO: 358 - protein, 17 aa, Homo sapiens
YINAVNGNTQYSQKFQG

SEQ ID NO: 359 - protein, 16 aa, Homo sapiens
SMHHSGSSYYKPSLKS

SEQ ID NO: 360 - protein, 17 aa, Homo sapiens
VISNDGSNKYYADSVKG

SEQ ID NO: 361 - protein, 16 aa, Homo sapiens
RIDWDDDKYYSTSLKT

SEQ ID NO: 362 - protein, 16 aa, Homo sapiens
SIFYSGTTYYNPSLKS

SEQ ID NO: 363 - protein, 17 aa, Homo sapiens
WISAYNGNTFYAQRLQG

SEQ ID NO: 364 - protein, 17 aa, Homo sapiens
IINPSGGSTTYAQTFQD

SEQ ID NO: 365 - protein, 17 aa, Homo sapiens
VVSYDGNHNDYADSVKG

SEQ ID NO: 366 - protein, 19 aa, Homo sapiens
LIKSHFEGGATDYAAPVKG

SEQ ID NO: 367 - protein, 17 aa, Homo sapiens
VISYDGAKKFYANSVKG

SEQ ID NO: 368 - protein, 17 aa, Homo sapiens
VIWHDGSNIRYADSVRG

SEQ ID NO: 369 - protein, 16 aa, Homo sapiens
SVHHSGSTYYNPSLKS

SEQ ID NO: 370 - protein, 17 aa, Homo sapiens
ILNPDGGTTFYAEKFQD

SEQ ID NO: 371 - protein, 18 aa, Homo sapiens
CARDVGYGGGQYFAMDVW

SEQ ID NO: 372 - protein, 24 aa, Homo sapiens
CAKDMDYYGSRSYSVTYYYGMDVW

SEQ ID NO: 373 - protein, 24 aa, Homo sapiens
CARRGAVALVPAAEDPYYYGMDVW

SEQ ID NO: 374 - protein, 20 aa, Homo sapiens
CAREDGTMGTNSWYGWFDPW

SEQ ID NO: 375 - protein, 22 aa, Homo sapiens
CARGLILALPTATVELGAFDIW

SEQ ID NO: 376 - protein, 26 aa, Homo sapiens
CARSYRSQTDILTGRYKGPGDVFDNW

SEQ ID NO: 377 - protein, 20 aa, Homo sapiens
CARDVDDFPVWGMNRYLALW

SEQ ID NO: 378 - protein, 19 aa, Homo sapiens
CAKDDVATDLAAYYYFDVW

SEQ ID NO: 379 - protein, 20 aa, Homo sapiens
CVRGGVVTNRVYYYYGMDVW

SEQ ID NO: 380 - protein, 13 aa, Homo sapiens
CAWFGEFGLFDYW

SEQ ID NO: 381 - protein, 18 aa, Homo sapiens
CARGSVQVWLHLGLFDNW

SEQ ID NO: 382 - protein, 17 aa, Homo sapiens
CARTPYEFWSGYYFDFW

SEQ ID NO: 383 - protein, 11 aa, Homo sapiens
CARKWLGMDFW

SEQ ID NO: 384 - protein, 13 aa, Homo sapiens
CARARPGYKVDFW

SEQ ID NO: 385 - protein, 17 aa, Homo sapiens
CARGGTLYTTGGEMHIW

SEQ ID NO: 386 - protein, 15 aa, Homo sapiens
CARRFWGFGNFFDYW

SEQ ID NO: 387 - protein, 20 aa, Homo sapiens
CAREGHHSGSGDYYSFFDYW

SEQ ID NO: 388 - protein, 22 aa, Homo sapiens
CVRRGGFCTATGCYAGHWFDPW

SEQ ID NO: 389 - protein, 20 aa, Homo sapiens
CARIVFHTSGGYYNPYMDVW

SEQ ID NO: 390 - protein, 15 aa, Homo sapiens
CARRAYDSGWHFEHW

SEQ ID NO: 391 - protein, 17 aa, Homo sapiens
CLRGSTRGWDTDGFDIW

SEQ ID NO: 392 - protein, 24 aa, Homo sapiens
CARQRSVTGGFDAWLLIPDASNTW

SEQ ID NO: 393 - protein, 21 aa, Homo sapiens
CARVFREFSTSTLDPYYFDYW

SEQ ID NO: 394 - protein, 22 aa, Homo sapiens
CVRQGGYYDRNGYHEKYAFDIW

SEQ ID NO: 395 - protein, 20 aa, Homo sapiens
CARAGRSSMNEEVIMYFDNW

SEQ ID NO: 396 - protein, 22 aa, Homo sapiens
CANIGQRRYCSGDHCYGHFDYW

SEQ ID NO: 397 - protein, 22 aa, Homo sapiens
CAKDHIGGTNAYFEWTVPFDGW

SEQ ID NO: 398 - protein, 20 aa, Homo sapiens
CARTQVFASGGYYLYYLDHW

SEQ ID NO: 399 - protein, 23 aa, Homo sapiens
CARDLGYGGNSYSHSYYYGLDVW

SEQ ID NO: 400 - protein, 11 aa, Homo sapiens
CARQGRGFGLW

SEQ ID NO: 401 - protein, 12 aa, Homo sapiens
CARVHGGGFDHW

SEQ ID NO: 402 - protein, 15 aa, Homo sapiens
CARDSSNWPAGYEDW

SEQ ID NO: 403 - protein, 18 aa, Homo sapiens
CAKDGGTYVPYSDAFDFW

SEQ ID NO: 404 - protein, 13 aa, Homo sapiens
CATVAAAGNFDNW

SEQ ID NO: 405 - protein, 16 aa, Homo sapiens
CARIAITMVRNPFDIW

SEQ ID NO: 406 - protein, 20 aa, Homo sapiens
CARTGIYDSSGYYLYYFDYW

SEQ ID NO: 407 - protein, 25 aa, Homo sapiens
CARDRVGGSSSEVLSRAKNYGLDVW

SEQ ID NO: 408 - protein, 19 aa, Homo sapiens
CARRASQYGEVYGNYFDYW

SEQ ID NO: 409 - protein, 21 aa, Homo sapiens
CAKDDFGNSNGVFFMSRVAFW

SEQ ID NO: 410 - protein, 21 aa, Homo sapiens
CVRGFNEQQLVPGLSFWFDYW

SEQ ID NO: 411 - protein, 21 aa, Homo sapiens
CARDRNVVLLPAAPFGGMDVW

SEQ ID NO: 412 - protein, 13 aa, Homo sapiens
CARGSPGDAFDIW

SEQ ID NO: 413 - protein, 18 aa, Homo sapiens
CAAQTPYFNESSGLVPDW

SEQ ID NO: 414 - protein, 18 aa, Homo sapiens
CARDLGDGYTAWGWFDPW

SEQ ID NO: 415 - protein, 21 aa, Homo sapiens
CTRDESMLRGVTEGFGPIDYW

SEQ ID NO: 416 - protein, 18 aa, Homo sapiens
CVISFDSTIAAAEYFDYW

SEQ ID NO: 417 - protein, 22 aa, Homo sapiens
CAREGHYSGSSSYQRDDAFDIW

SEQ ID NO: 418 - protein, 23 aa, Homo sapiens
CARGGTIEATPEREYYYYGMDVW

SEQ ID NO: 419 - protein, 13 aa, Homo sapiens
CASRSFYGDYVYW

SEQ ID NO: 420 - protein, 13 aa, Homo sapiens
CAKEGSGWYFESW

SEQ ID NO: 421 - protein, 18 aa, Homo sapiens
CTRHVGEMSTIWWYFDLW

SEQ ID NO: 422 - protein, 19 aa, Homo sapiens
CAKSGSHYGEVYGAYFDYW

SEQ ID NO: 423 - protein, 20 aa, Homo sapiens
CARDRGPGYSDSSFYVFDYW

SEQ ID NO: 424 - protein, 18 aa, Homo sapiens
CTRAPRGSTASHLLFDYW

SEQ ID NO: 425 - protein, 23 aa, Homo sapiens
CARPKYYFDSSGQFSEMYYFDFW

SEQ ID NO: 426 - protein, 14 aa, Homo sapiens
CARDLLRSTYFDYW

SEQ ID NO: 427 - protein, 21 aa, Homo sapiens
CARDGNTAGVDMWSRDGFDIW

SEQ ID NO: 428 - protein, 26 aa, Homo sapiens
CAKEPWIDIVVASVISPYYYDGMDVW

SEQ ID NO: 429 - protein, 18 aa, Homo sapiens
CARMNLGSHSGRPGFDMW

SEQ ID NO: 430 - protein, 24 aa, Homo sapiens
CATGGGVNVTSWSDVEHSSSLGYW

SEQ ID NO: 431 - protein, 20 aa, Homo sapiens
CVKDEVYDSSGYYLYYFDSW

SEQ ID NO: 432 - protein, 22 aa, Homo sapiens
CAKDYDFWSGYPGGQYWFFDLW

SEQ ID NO: 433 - protein, 21 aa, Homo sapiens
CVRGGTYSSDVEYYYYGMDVW

SEQ ID NO: 434 - protein, 18 aa, Homo sapiens
CARLTLGSYTGRPGFDSW

SEQ ID NO: 435 - protein, 18 aa, Homo sapiens
CARDTILTFGEPHWFDPW

SEQ ID NO: 436 - protein, 19 aa, Homo sapiens
CARDLRYLTYYSGSGDDSW

SEQ ID NO: 437 - protein, 20 aa, Homo sapiens
CARGLFYDSGGYYLFYFQHW

SEQ ID NO: 438 - protein, 20 aa, Homo sapiens
CARASEYSISWRHRGVLDYW

SEQ ID NO: 439 - protein, 19 aa, Homo sapiens
CHGEGYSTSWLGTAALDYW

SEQ ID NO: 440 - protein, 18 aa, Homo sapiens
CAKTRGYSYTWGDAFDLW

SEQ ID NO: 441 - protein, 20 aa, Homo sapiens
CAHSAYYTSSGYYLQYFHHW

SEQ ID NO: 442 - protein, 20 aa, Homo sapiens
CARSDYYDSSGYYLLYLDSW

SEQ ID NO: 443 - protein, 15 aa, Homo sapiens
CARNNGGSAIIFYYW

SEQ ID NO: 444 - protein, 20 aa, Homo sapiens
CARDLVVVTDISIKNYFDPW

SEQ ID NO: 445 - protein, 17 aa, Homo sapiens
CAKTTDQRLLVDWFDPW

SEQ ID NO: 446 - protein, 20 aa, Homo sapiens
CARTLVYAPDSYYLYYFDYW

SEQ ID NO: 447 - protein, 24 aa, Homo sapiens
CARHGFRYCNNGVCSINLDAFDIW

SEQ ID NO: 448 - protein, 21 aa, Homo sapiens
CARDLRMLPGGLPTRRGMDVW

SEQ ID NO: 449 - protein, 25 aa, Homo sapiens
CARGIREGGVSVEDWMLVYSWFDPW

SEQ ID NO: 450 - protein, 13 aa, Homo sapiens
CVRAPGSMGLDVW

SEQ ID NO: 451 - protein, 13 aa, Homo sapiens
CAPLGGPTPFDYW

SEQ ID NO: 452 - protein, 15 aa, Homo sapiens
CATASTYFYDSRDYW

SEQ ID NO: 453 - protein, 17 aa, Homo sapiens
CARVPFQIWSGLYFDHW

SEQ ID NO: 454 - protein, 18 aa, Homo sapiens
CARDRVALGVHYWYFDIW

SEQ ID NO: 455 - protein, 23 aa, Homo sapiens
CAILIARAYCGLADGQEGDFDTW

SEQ ID NO: 456 - protein, 11 aa, Homo sapiens
RASQSVNSHLA

SEQ ID NO: 457 - protein, 11 aa, Homo sapiens
RASQRISNHLN

SEQ ID NO: 458 - protein, 16 aa, Homo sapiens
RSSQSLLHSNGNNYLD

SEQ ID NO: 459 - protein, 12 aa, Homo sapiens
RASQSVSSSYLA

SEQ ID NO: 460 - protein, 11 aa, Homo sapiens
RASQSITGYLN

SEQ ID NO: 461 - protein, 11 aa, Homo sapiens
RASEGISSWLA

SEQ ID NO: 462 - protein, 12 aa, Homo sapiens
RASQSVSSSYLA

SEQ ID NO: 463 - protein, 16 aa, Homo sapiens
RSSQSLLRSDGKTFLY

SEQ ID NO: 464 - protein, 11 aa, Homo sapiens
RASQGISSYLA

SEQ ID NO: 465 - protein, 11 aa, Homo sapiens
RASQDINNYLA

SEQ ID NO: 466 - protein, 11 aa, Homo sapiens
RASQSVSSWVA

SEQ ID NO: 467 - protein, 11 aa, Homo sapiens
RASQGITDSLA

SEQ ID NO: 468 - protein, 16 aa, Homo sapiens
RSSQSLLNSNGFNYVD

SEQ ID NO: 469 - protein, 11 aa, Homo sapiens
RASQGISSYLA

SEQ ID NO: 470 - protein, 12 aa, Homo sapiens
RASQTVSSSYLV

SEQ ID NO: 471 - protein, 12 aa, Homo sapiens
RASQSVSSGYLA

SEQ ID NO: 472 - protein, 11 aa, Homo sapiens
RASQGINTYLN

SEQ ID NO: 473 - protein, 12 aa, Homo sapiens
RASQSISSGYLA

SEQ ID NO: 474 - protein, 11 aa, Homo sapiens
RASQTIASYLS

SEQ ID NO: 475 - protein, 11 aa, Homo sapiens
RASQSVGSKLA

SEQ ID NO: 476 - protein, 11 aa, Homo sapiens
RASQGISNYLV

SEQ ID NO: 477 - protein, 17 aa, Homo sapiens
RSSETVLYTSKNQSYLA

SEQ ID NO: 478 - protein, 12 aa, Homo sapiens
RASQSVSSSYIA

SEQ ID NO: 479 - protein, 11 aa, Homo sapiens
RASQSISSWLA

SEQ ID NO: 480 - protein, 11 aa, Homo sapiens
RASQSIGSRLA

SEQ ID NO: 481 - protein, 16 aa, Homo sapiens
RSSQSLLHSDGRYYVD

SEQ ID NO: 482 - protein, 11 aa, Homo sapiens
WASQTIGGNLA

SEQ ID NO: 483 - protein, 11 aa, Homo sapiens
RASQTIASYVN

SEQ ID NO: 484 - protein, 11 aa, Homo sapiens
RASQSVSSSLA

SEQ ID NO: 485 - protein, 11 aa, Homo sapiens
QASQDITYYLS

SEQ ID NO: 486 - protein, 11 aa, Homo sapiens
QASQDIGDSLN

SEQ ID NO: 487 - protein, 11 aa, Homo sapiens
RPSQDISSALA

SEQ ID NO: 488 - protein, 17 aa, Homo sapiens
KSSQSVLYNSNNKNYLA

SEQ ID NO: 489 - protein, 11 aa, Homo sapiens
RASQFISSYLH

SEQ ID NO: 490 - protein, 11 aa, Homo sapiens
RASQSIGSWLA

SEQ ID NO: 491 - protein, 11 aa, Homo sapiens
RASQSIASYLN

SEQ ID NO: 492 - protein, 11 aa, Homo sapiens
RASQSVTSELA

SEQ ID NO: 493 - protein, 11 aa, Homo sapiens
RASQNIYNWLA

SEQ ID NO: 494 - protein, 11 aa, Homo sapiens
RANQDIDNYLA

SEQ ID NO: 495 - protein, 11 aa, Homo sapiens
RASQGISKRLA

SEQ ID NO: 496 - protein, 11 aa, Homo sapiens
RASQGISSYLA

SEQ ID NO: 497 - protein, 11 aa, Homo sapiens
RASQGIGTWLA

SEQ ID NO: 498 - protein, 11 aa, Homo sapiens
RASQGISNYLA

SEQ ID NO: 499 - protein, 12 aa, Homo sapiens
RASQSVGGRSLA

SEQ ID NO: 500 - protein, 17 aa, Homo sapiens
RSSQSVLYSSNNKNYLA

SEQ ID NO: 501 - protein, 11 aa, Homo sapiens
RASQTISNSLA

SEQ ID NO: 502 - protein, 11 aa, Homo sapiens
RASQGISNYLA

SEQ ID NO: 503 - protein, 11 aa, Homo sapiens
RASQGISSYLA

SEQ ID NO: 504 - protein, 12 aa, Homo sapiens
RASQSVSSSYLA

SEQ ID NO: 505 - protein, 11 aa, Homo sapiens
RASQGISAWLA

SEQ ID NO: 506 - protein, 11 aa, Homo sapiens
RASQSISSYLN

SEQ ID NO: 507 - protein, 11 aa, Homo sapiens
RASQNIYNWLA

SEQ ID NO: 508 - protein, 16 aa, Homo sapiens
RSSQSLVNSDGNTYLS

SEQ ID NO: 509 - protein, 11 aa, Homo sapiens
QASQDVSYYLN

SEQ ID NO: 510 - protein, 12 aa, Homo sapiens
RASQSVSSNYLA

SEQ ID NO: 511 - protein, 11 aa, Homo sapiens
RASQAISNWLA

SEQ ID NO: 512 - protein, 17 aa, Homo sapiens
RSSQSLLDSNDGNTYLD

SEQ ID NO: 513 - protein, 16 aa, Homo sapiens
RSSQSLLHRNEYNYLD

SEQ ID NO: 514 - protein, 11 aa, Homo sapiens
QASQDISNYLN

SEQ ID NO: 515 - protein, 11 aa, Homo sapiens
RASQGIRNYLA

SEQ ID NO: 516 - protein, 11 aa, Homo sapiens
RASQIIASYLN

SEQ ID NO: 517 - protein, 11 aa, Homo sapiens
RTSQSVSSYLA

SEQ ID NO: 518 - protein, 11 aa, Homo sapiens
RASQGISIYLA

SEQ ID NO: 519 - protein, 11 aa, Homo sapiens
QASQDINNYLN

SEQ ID NO: 520 - protein, 11 aa, Homo sapiens
RASQSIKNNLA

SEQ ID NO: 521 - protein, 12 aa, Homo sapiens
RASQSLSDNYLA

SEQ ID NO: 522 - protein, 11 aa, Homo sapiens
RASQRIASYLN

SEQ ID NO: 523 - protein, 11 aa, Homo sapiens
QASQGISNYLN

SEQ ID NO: 524 - protein, 11 aa, Homo sapiens
RASQGIRNFLA

SEQ ID NO: 525 - protein, 11 aa, Homo sapiens
RASQSVTSNLA

SEQ ID NO: 526 - protein, 11 aa, Homo sapiens
RASQTIASYVN

SEQ ID NO: 527 - protein, 11 aa, Homo sapiens
RASQTIASYVN

SEQ ID NO: 528 - protein, 11 aa, Homo sapiens
RSSQTISVFLN

SEQ ID NO: 529 - protein, 11 aa, Homo sapiens
RASQSVTKYLA

SEQ ID NO: 530 - protein, 11 aa, Homo sapiens
RASQSVSSNLA

SEQ ID NO: 531 - protein, 11 aa, Homo sapiens
RASQTIASYVN

SEQ ID NO: 532 - protein, 16 aa, Homo sapiens
RSSQSLLRTNGYNYLD

SEQ ID NO: 533 - protein, 11 aa, Homo sapiens
RASQSISSWLA

SEQ ID NO: 534 - protein, 11 aa, Homo sapiens
RASQNIRTFIN

SEQ ID NO: 535 - protein, 16 aa, Homo sapiens
RSSQSLLHRNGYNHLD

SEQ ID NO: 536 - protein, 11 aa, Homo sapiens
RAGQGIRNDLG

SEQ ID NO: 537 - protein, 16 aa, Homo sapiens
RSSRSLVHSDGNTYLS

SEQ ID NO: 538 - protein, 11 aa, Homo sapiens
RASQSVGNNLA

SEQ ID NO: 539 - protein, 11 aa, Homo sapiens
RASQSVSSHLA

SEQ ID NO: 540 - protein, 11 aa, Homo sapiens
RASRSITSWLA

SEQ ID NO: 541 - protein, 7 aa, Homo sapiens
NTFNRVT

SEQ ID NO: 542 - protein, 7 aa, Homo sapiens
GASTLQS

SEQ ID NO: 543 - protein, 7 aa, Homo sapiens
LASNRAS

SEQ ID NO: 544 - protein, 7 aa, Homo sapiens
GASSRAT

SEQ ID NO: 545 - protein, 7 aa, Homo sapiens
ATSTLQS

SEQ ID NO: 546 - protein, 7 aa, Homo sapiens
AASTLQS

SEQ ID NO: 547 - protein, 7 aa, Homo sapiens
GASTGAT

SEQ ID NO: 548 - protein, 7 aa, Homo sapiens
EVSSRFS

SEQ ID NO: 549 - protein, 7 aa, Homo sapiens
AASTLQS

SEQ ID NO: 550 - protein, 7 aa, Homo sapiens
AASSLQS

SEQ ID NO: 551 - protein, 7 aa, Homo sapiens
EASNLES

SEQ ID NO: 552 - protein, 7 aa, Homo sapiens
AASRLES

SEQ ID NO: 553 - protein, 7 aa, Homo sapiens
LGSNRAS

SEQ ID NO: 554 - protein, 7 aa, Homo sapiens
VASILES

SEQ ID NO: 555 - protein, 7 aa, Homo sapiens
GASTRAT

SEQ ID NO: 556 - protein, 7 aa, Homo sapiens
GASGRAT

SEQ ID NO: 557 - protein, 7 aa, Homo sapiens
AASSLQS

SEQ ID NO: 558 - protein, 7 aa, Homo sapiens
GASHRAT

SEQ ID NO: 559 - protein, 7 aa, Homo sapiens
TASSLQS

SEQ ID NO: 560 - protein, 7 aa, Homo sapiens
GASTRAT

SEQ ID NO: 561 - protein, 7 aa, Homo sapiens
AASSLQS

SEQ ID NO: 562 - protein, 7 aa, Homo sapiens
WASTRES

SEQ ID NO: 563 - protein, 7 aa, Homo sapiens
AASRRAT

SEQ ID NO: 564 - protein, 7 aa, Homo sapiens
KSSILES

SEQ ID NO: 565 - protein, 7 aa, Homo sapiens
DASSLES

SEQ ID NO: 566 - protein, 7 aa, Homo sapiens
LASNRAS

SEQ ID NO: 567 - protein, 7 aa, Homo sapiens
GASTRAT

SEQ ID NO: 568 - protein, 7 aa, Homo sapiens
AASNLQS

SEQ ID NO: 569 - protein, 7 aa, Homo sapiens
DASYRVT

SEQ ID NO: 570 - protein, 7 aa, Homo sapiens
DVSNLER

SEQ ID NO: 571 - protein, 7 aa, Homo sapiens
DASNLET

SEQ ID NO: 572 - protein, 7 aa, Homo sapiens
GASTLDY

SEQ ID NO: 573 - protein, 7 aa, Homo sapiens
LASTREY

SEQ ID NO: 574 - protein, 7 aa, Homo sapiens
AASTLQS

SEQ ID NO: 575 - protein, 7 aa, Homo sapiens
KESNLES

SEQ ID NO: 576 - protein, 7 aa, Homo sapiens
AASSLHS

SEQ ID NO: 577 - protein, 7 aa, Homo sapiens
KASSLES

SEQ ID NO: 578 - protein, 7 aa, Homo sapiens
DASTLES

SEQ ID NO: 579 - protein, 7 aa, Homo sapiens
GASKLQT

SEQ ID NO: 580 - protein, 7 aa, Homo sapiens
GASSLQH

SEQ ID NO: 581 - protein, 7 aa, Homo sapiens
AASTLQS

SEQ ID NO: 582 - protein, 7 aa, Homo sapiens
AASRLQS

SEQ ID NO: 583 - protein, 7 aa, Homo sapiens
AASTLQS

SEQ ID NO: 584 - protein, 7 aa, Homo sapiens
DASNRAT

SEQ ID NO: 585 - protein, 7 aa, Homo sapiens
WASTRAS

SEQ ID NO: 586 - protein, 7 aa, Homo sapiens
KASTLES

SEQ ID NO: 587 - protein, 7 aa, Homo sapiens
TTSTLRS

SEQ ID NO: 588 - protein, 7 aa, Homo sapiens
AASTLQS

SEQ ID NO: 589 - protein, 7 aa, Homo sapiens
GASSRAT

SEQ ID NO: 590 - protein, 7 aa, Homo sapiens
DASTLAS

SEQ ID NO: 591 - protein, 7 aa, Homo sapiens
AASSLQS

SEQ ID NO: 592 - protein, 7 aa, Homo sapiens
DASSLES

SEQ ID NO: 593 - protein, 7 aa, Homo sapiens
QISKRFS

SEQ ID NO: 594 - protein, 7 aa, Homo sapiens
DTSNLVT

SEQ ID NO: 595 - protein, 7 aa, Homo sapiens
GASSRAA

SEQ ID NO: 596 - protein, 7 aa, Homo sapiens
AASSLQS

SEQ ID NO: 597 - protein, 7 aa, Homo sapiens
TFSYRAS

SEQ ID NO: 598 - protein, 7 aa, Homo sapiens
WGSNRAS

SEQ ID NO: 599 - protein, 7 aa, Homo sapiens
DATKLET

SEQ ID NO: 600 - protein, 7 aa, Homo sapiens
AASTLQS

SEQ ID NO: 601 - protein, 7 aa, Homo sapiens
AASSLQS

SEQ ID NO: 602 - protein, 7 aa, Homo sapiens
DASNRAT

SEQ ID NO: 603 - protein, 7 aa, Homo sapiens
AASTLQT

SEQ ID NO: 604 - protein, 7 aa, Homo sapiens
DATDLET

SEQ ID NO: 605 - protein, 7 aa, Homo sapiens
GASARAT

SEQ ID NO: 606 - protein, 7 aa, Homo sapiens
GASSRPT

SEQ ID NO: 607 - protein, 7 aa, Homo sapiens
AASSLQS

SEQ ID NO: 608 - protein, 7 aa, Homo sapiens
DASNLES

SEQ ID NO: 609 - protein, 7 aa, Homo sapiens
AASTLQS

SEQ ID NO: 610 - protein, 7 aa, Homo sapiens
GASTRAT

SEQ ID NO: 611 - protein, 7 aa, Homo sapiens
AASSLQS

SEQ ID NO: 612 - protein, 7 aa, Homo sapiens
AASNLQS

SEQ ID NO: 613 - protein, 7 aa, Homo sapiens
AASSLHS

SEQ ID NO: 614 - protein, 7 aa, Homo sapiens
DASNRAT

SEQ ID NO: 615 - protein, 7 aa, Homo sapiens
SASTRAT

SEQ ID NO: 616 - protein, 7 aa, Homo sapiens
AASRLQS

SEQ ID NO: 617 - protein, 7 aa, Homo sapiens
LGSIRAS

SEQ ID NO: 618 - protein, 7 aa, Homo sapiens
KASSLES

SEQ ID NO: 619 - protein, 7 aa, Homo sapiens
AASKLES

SEQ ID NO: 620 - protein, 7 aa, Homo sapiens
LGSNRAS

SEQ ID NO: 621 - protein, 7 aa, Homo sapiens
GASTLQS

SEQ ID NO: 622 - protein, 7 aa, Homo sapiens
KISNRFS

SEQ ID NO: 623 - protein, 7 aa, Homo sapiens
GASTRAT

SEQ ID NO: 624 - protein, 7 aa, Homo sapiens
GASTRAT

SEQ ID NO: 625 - protein, 7 aa, Homo sapiens
KASSLQS

SEQ ID NO: 626 - protein, 13 aa, Homo sapiens [sequence omitted]

SEQ ID NO: 627 - protein, 12 aa, Homo sapiens
CQQSYRTPPINF

SEQ ID NO: 628 - protein, 10 aa, Homo sapiens [sequence omitted]

SEQ ID NO: 629 - protein, 13 aa, Homo sapiens
CQQYDSSLSTWTF

SEQ ID NO: 630 - protein, 10 aa, Homo sapiens [sequence omitted]

SEQ ID NO: 631 - protein, 11 aa, Homo sapiens [sequence omitted]

SEQ ID NO: 632 - protein, 11 aa, Homo sapiens [sequence omitted]

SEQ ID NO: 633 - protein, 11 aa, Homo sapiens
CMQGLKIRRTF

SEQ ID NO: 634 - protein, 11 aa, Homo sapiens
CQQVDTYPLTF

SEQ ID NO: 635 - protein, 11 aa, Homo sapiens [sequence omitted]

SEQ ID NO: 636 - protein, 12 aa, Homo sapiens
CQQYHSYSGYTF

SEQ ID NO: 637 - protein, 11 aa, Homo sapiens
CQQYSKSPATF

SEQ ID NO: 638 - protein, 11 aa, Homo sapiens [sequence omitted]

SEQ ID NO: 639 - protein, 11 aa, Homo sapiens
CQQSKSFPPTF

SEQ ID NO: 640 - protein, 11 aa, Homo sapiens
CQQYGGSGLTF

SEQ ID NO: 641 - protein, 11 aa, Homo sapiens
CQQYFGSPYTF

SEQ ID NO: 642 - protein, 11 aa, Homo sapiens
CQQSANSPHTF

SEQ ID NO: 643 - protein, 11 aa, Homo sapiens [sequence omitted]

SEQ ID NO: 644 - protein, 11 aa, Homo sapiens
CQHSYNTPYTF

SEQ ID NO: 645 - protein, 12 aa, Homo sapiens [sequence omitted]

SEQ ID NO: 646 - protein, 11 aa, Homo sapiens
CLQHNISPYTF

SEQ ID NO: 647 - protein, 11 aa, Homo sapiens
CQQFFRSPFTF

SEQ ID NO: 648 - protein, 11 aa, Homo sapiens [sequence omitted]

SEQ ID NO: 649 - protein, 11 aa, Homo sapiens [sequence omitted]

SEQ ID NO: 650 - protein, 12 aa, Homo sapiens [sequence omitted]

SEQ ID NO: 651 - protein, 11 aa, Homo sapiens [sequence omitted]

SEQ ID NO: 652 - protein, 10 aa, Homo sapiens [sequence omitted]

SEQ ID NO: 653 - protein, 12 aa, Homo sapiens
CQQSYSYRALTF

SEQ ID NO: 654 - protein, 13 aa, Homo sapiens [sequence omitted]

SEQ ID NO: 655 - protein, 11 aa, Homo sapiens
CQQYDFLPYTF

SEQ ID NO: 656 - protein, 13 aa, Homo sapiens
CQHYVNLPPSFTF

SEQ ID NO: 657 - protein, 11 aa, Homo sapiens [sequence omitted]

SEQ ID NO: 658 - protein, 11 aa, Homo sapiens
CQQYYQTPLTF

SEQ ID NO: 659 - protein, 11 aa, Homo sapiens [sequence omitted]

SEQ ID NO: 660 - protein, 10 aa, Homo sapiens
CQQYKNDWTF

SEQ ID NO: 661 - protein, 11 aa, Homo sapiens
CQHSYSTRFTF

SEQ ID NO: 662 - protein, 11 aa, Homo sapiens [sequence omitted]

SEQ ID NO: 663 - protein, 11 aa, Homo sapiens [sequence omitted]

SEQ ID NO: 664 - protein, 11 aa, Homo sapiens [sequence omitted]

SEQ ID NO: 665 - protein, 11 aa, Homo sapiens [sequence omitted]

SEQ ID NO: 666 - protein, 11 aa, Homo sapiens [sequence omitted]

SEQ ID NO: 667 - protein, 11 aa, Homo sapiens [sequence omitted]

SEQ ID NO: 668 - protein, 11 aa, Homo sapiens [sequence omitted]

SEQ ID NO: 669 - protein, 11 aa, Homo sapiens [sequence omitted]

SEQ ID NO: 670 - protein, 11 aa, Homo sapiens
CQQFHSTPRTF

SEQ ID NO: 671 - protein, 11 aa, Homo sapiens
CQQYNSFSFTF

SEQ ID NO: 672 - protein, 11 aa, Homo sapiens [sequence omitted]

SEQ ID NO: 673 - protein, 11 aa, Homo sapiens [sequence omitted]

SEQ ID NO: 674 - protein, 11 aa, Homo sapiens [sequence omitted]

SEQ ID NO: 675 - protein, 11 aa, Homo sapiens [sequence omitted]

SEQ ID NO: 676 - protein, 11 aa, Homo sapiens [sequence omitted]

SEQ ID NO: 677 - protein, 11 aa, Homo sapiens
CQQYNIYSPTF

SEQ ID NO: 678 - protein, 11 aa, Homo sapiens [sequence omitted]

SEQ ID NO: 679 - protein, 11 aa, Homo sapiens
CLQYHYLPYTF

SEQ ID NO: 680 - protein, 11 aa, Homo sapiens [sequence omitted]

SEQ ID NO: 681 - protein, 11 aa, Homo sapiens
CQQADTFPFTF

SEQ ID NO: 682 - protein, 11 aa, Homo sapiens [sequence omitted]

SEQ ID NO: 683 - protein, 11 aa, Homo sapiens [sequence omitted]

SEQ ID NO: 684 - protein, 11 aa, Homo sapiens
CQHFANLPYTF

SEQ ID NO: 685 - protein, 11 aa, Homo sapiens [sequence omitted]

SEQ ID NO: 686 - protein, 12 aa, Homo sapiens [sequence omitted]

SEQ ID NO: 687 - protein, 10 aa, Homo sapiens [sequence omitted]

SEQ ID NO: 688 - protein, 11 aa, Homo sapiens [sequence omitted]

SEQ ID NO: 689 - protein, 11 aa, Homo sapiens
CQHFANLPYTF

SEQ ID NO: 690 - protein, 12 aa, Homo sapiens
CQEYNNWPLLTF

SEQ ID NO: 691 - protein, 11 aa, Homo sapiens [sequence omitted]

SEQ ID NO: 692 - protein, 12 aa, Homo sapiens
CQQSYSTPIYTF

SEQ ID NO: 693 - protein, 11 aa, Homo sapiens [sequence omitted]

SEQ ID NO: 694 - protein, 11 aa, Homo sapiens [sequence omitted]

SEQ ID NO: 695 - protein, 11 aa, Homo sapiens
CQQYNNWPQTF

SEQ ID NO: 696 - protein, 11 aa, Homo sapiens [sequence omitted]

SEQ ID NO: 697 - protein, 12 aa, Homo sapiens
CQQSYSVPRLTF

SEQ ID NO: 698 - protein, 10 aa, Homo sapiens
CQESFSSSTF

SEQ ID NO: 699 - protein, 10 aa, Homo sapiens
CQHRRSWPTF

SEQ ID NO: 700 - protein, 12 aa, Homo sapiens
CQQYNMWPPWTF

SEQ ID NO: 701 - protein, 11 aa, Homo sapiens [sequence omitted]

SEQ ID NO: 702 - protein, 11 aa, Homo sapiens
CMQSLQTSITF

SEQ ID NO: 703 - protein, 11 aa, Homo sapiens [sequence omitted]

SEQ ID NO: 704 - protein, 11 aa, Homo sapiens
CQQGHSTPYTF

SEQ ID NO: 705 - protein, 11 aa, Homo sapiens [sequence omitted]

SEQ ID NO: 706 - protein, 11 aa, Homo sapiens [sequence omitted]

SEQ ID NO: 707 - protein, 10 aa, Homo sapiens
CLQATQFLTF

SEQ ID NO: 708 - protein, 11 aa, Homo sapiens
CQQYDKWPETF

SEQ ID NO: 709 - protein, 11 aa, Homo sapiens
CQQYDNWLPTF

SEQ ID NO: 710 - protein, 11 aa, Homo sapiens [sequence omitted]

SEQ ID NO: 711 - protein, 298 aa, respiratory syncytial virus
MSKNKDQRTAKTLEKTWDTLNHLLFISSGLYKLNLKSIAQITLSILAMIISTSLIITAIIFIASANHKVTLTTAIIQDATSQIKNTTPTYLTQDPQLGISFSNLSEITSQTTTILASTTPGVKSNLQPTTVKTKNTTTTQTQPSKPTTKQRQNKPPNKPNNDFHFEVFNFVPCSICSNNPTCWAICKRIPNKKPGKKTTTKPTKKPTFKTTKKDHKPQTTKPKEVPTTKPTEEPTINTTKTNIITTLLTNNTTGNPKLTSQMETFHSTSSEGNLSPSQVSTTSEHPSQPSSPPNTTRQ

SEQ ID NO: 712 - protein, 292 aa, respiratory syncytial virus
MSKHKNQRTARTLEKTWDTLNHLIVISSCLYRLNLKSIAQIALSVLAMIISTSLIIAAIIFIISANHKVTLTTVTVQTIKNHTEKNISTYLTQVPPERVNSSKQPTTTSPIHTNSATISPNTKSETHHTTAQTKGRITTSTQTNKPSTKSRSKNPPKKPKDDYHFEVFNFVPCSICGNNQLCKSICKTIPSNKPKKKPTIKPTNKPTTKTTNKRDPKTPAKMPKKEIITNPAKKPTLKTTERDTSISQSTVLDTITPKYTIQQQSLHSTTSENTPSSTQIPTASEPSTLNPN

SEQ ID NO: 713 - protein, 77 aa, respiratory syncytial virus
QPTTVKTKNTTTTQTQPSKPTTKQRQNKPPNKPNNDFHFEVFNFVPCSICSNNPTCWAICKRIPNKKPGKKTTTKPT

SEQ ID NO: 714 - protein, 77 aa, respiratory syncytial virus
HHTTAQTKGRITTSTQTNKPSTKSRSKNPPKKPKDDYHFEVFNFVPCSICGNNQLCKSICKTIPSNKPKKKPTIKPT